SEQUENCE LISTING (229 sequences)

SEQ ID NO: 1 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 398 aa, Homo sapiens
MPVPASWPHLPSPFLLMTLLLGRLTGVAGEDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPSAPVVSGPAVRATPEHTVSFTCESHGFSPRDITLKWFKNGNELSDFQTNVDPAGDSVSYSIHSTARVVLTRGDVHSQVICEIAHITLQGDPLRGTANLSEAIRVPPTLEVTQQPMRAENQANVTCQVSNFYPRGLQLTWLENGNVSRTETASTLIENKDGTYNWMSWLLVNTCAHRDDVVLTCQVEHDGQQAVSKSYALEISAHQKEHGSDITHEAALAPTAPLLVALLLGPKLLLVVGVSAIYICWKQKA

SEQ ID NO: 3 - protein, 387 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 4 - RNA, 4201 nt, Homo sapiens
  source 1..4201: mol_type = mRNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 5 - RNA, 5377 nt, Homo sapiens
  source 1..5377: mol_type = mRNA
agagcagagtctgcgatcttcccagcacagacgtttggacagagcaggctcctaaggtctccagaatgcccgtgccagcctcctggccccaccttcctagtcctttcctgctgatgacgctactgctggggagactcacaggagtggcaggtgaggacgagctacaggtgattcagcctgaaaagtccgtatcagttgcagctggagagtcggccactctgcgctgtgctatgacgtccctgatccctgtggggcccatcatgtggtttagaggagctggagcaggccgggaattaatctacaatcagaaagaaggccacttcccacgggtaacaactgtttcagaactcacaaagagaaacaacctggacttttccatcagcatcagtaacatcaccccagcagacgccggcacctactactgtgtgaagttccggaaagggagccctgacgacgtggagtttaagtctggagcaggcactgagctgtctgtgcgcgccaaaccctctgcccccgtggtatcgggccctgcggtgagggccacacctgagcacacagtgagcttcacctgcgagtcccatggcttctctcccagagacatcaccctgaaatggttcaaaaatgggaatgagctctcagacttccagaccaacgtggaccccgcaggagacagtgtgtcctacagcatccacagcacagccagggtggtgctgacccgtggggacgttcactctcaagtcatctgcgagatagcccacatcaccttgcagggggaccctcttcgtgggactgccaacttgtctgaggccatccgagttccacccaccttggaggttactcaacagcccatgagggcagagaaccaggcaaacgtcacctgccaggtgagcaatttctacccccggggactacagctgacctggttggagaatggaaatgtgtcccggacagaaacagcttcgaccctcatagagaacaaggatggcacctacaactggatgagctggctcctggtgaacacctgtgcccacagggacgatgtggtgctcacctgtcaggtggagcatgatgggcagcaagcagtcagcaaaagctatgccctggagatctcagcgcaccagaaggagcacggctcagatatcacccatgaagcagcgctggctcctactgctccactcctcgtagctctcctcctgggccccaagctgctactggtggttggtgtctctgccatctacatctgctggaaacagaaggcctgactgaccctcagtctctgctgcctcctcctttcttgagaagctcagcctgagagaaggagctggcgagaaccttccccacactcagctccaaacgcctcctctcccaggtcatctgcctgcccacacgctcctgttccaccttcacaagaccatgatgccccaaagcagtgtctctattcacggtcctgagcaggggccatgggattgggctctgggcactgactcatggcacctccctagaaggtgagaaacactccaaatctaaacacaccaggacttctcccatccgtcgccttgggactggccataaaccacagactctctccaggctctcaagagttatcctgtcttctggattcctgcctaccccaactcccccagccttgttgaggttctctactgcctcctgaatacacatgaacccctataccaattttaagaaaaaaatgattctctttcctctttgtccaagcatcctatccctcaaacccaaaaagaaagaagctctcccttctctctctgtgatggagacagtatttcttctagtatcctgcagccttcccagtcctgctgcttgtggtagaaatgctgccacagcccaacattgaggagccctcgatgactgccctttacaactcatattcagttctgcctccaaaatgcatgtgtccacttacatgagatggtaaatgtttaacaatggactttctgaaagggaaaaaccaaaagctgttttgcagtgcttgccaatttctctagtgtaataactcccaacctgaccaatttcacactgccaacagttaaacaaccagattgcaagattcctgaaatttaacaattggttttcagggcccagtccaagcctgctgctggaaacctcagagttaaatccctattctccacacctctcacctccaccacccctccctgtcccagccagcatcatctctttggggaccactcctctggctttcatttttcagccacagtgattctttggaaaagtcaaatcatatcacttctctgcttcttccccaacacagctgcatggctcccgctctccctccttcaagtctctgctcaatgtcacttcattaaaggcaccttctataaactaccttgtataaaatattatttattttctctatcccggcattctaatttctcttatcctaattaatttttctttagcccttattttgatgagtattatgccgaatacaggcagccctcacttttcatggcagtgcaagattgcaaaaatgactgtgcaacctgaaacccaggaaagcagtctccatagtcaatcagaaaaacaatgatcattctgtgacctttaccattttttgtcaaaatattagaaactctcacactctcagttacaaatgtagaggacaatgaaaatataatgaaataaatatttatttgtgcactacaattcaaagcattagaaacattgagagttcaagtgctgtttctttgtaaaaatgtatccagagtagttggaagagtgcttgcctttttttgtatatttctaatatggagtgatatagtttggctctgtgtctccatccaaatctcatcttaaattgtaatctgcatgtgttgtgggatgggcctggtaggaggtgactgaatcatgggggcggacttcccccttgctgttcttgtgatagtgagttctcataagatctcagtgagttctcatgagatctggttttttgaaagtgtgtggcaagtcccccttcgctctctctctctctctccctcctgccaccatgtgaagaaggtgcctgcttccttttctccttccaccatggttgtaagtttcctgaggcctcccagtcatgcttcctgttaagcctgtggaactgtgagtccaattaaacctcttttattcataaaatatccagtttctggtagttctttatagcagtgtgagaatgggctaatacacggagcaagcattgtttcttttcatttgtttattttatttttatttttttgagatggagtttcacccttattgcccaggctggagtgcaatgtcgtgatcttggctcactgcaacccccgcctccagggttcaagtgattctcctgcctcagcctcctgagtagctgggattacaggcatgtaccaccacacccagctaattttgtatttttagtagagatggggtttctccatgttgatcagactagtcttgaactcccgacctcaggtgatccacctgtcttggcctcccaaagtgctgggattacaggcatgagccaccatgcctagccagcaagcatcatttctattataccttggtgttttgccatctttctaagtttggactagcttccaacatcttatcccttgaattttcaatattgtggaatcactccagaagatcctttcatgtgaagttttttgctggcatttcaacctttgggacatcttcagcccttttattaccactcctctcccatttgtggcagtttgcgtttactacctccctctggctgcctatctgaagttcctgcatcagggtctacattgccacagtcaactatttgtacttctagaattccactcacattcaatttgaacctcacttgcagcattttcacttctcatttcttcactgcactttcatcttggttgcaaaattcctctttcaattattcatttctgtaaaatgtcatatgggctcatcactgggaggcaaggaggcaacccaactataggatttgctgtttgagtaggagagccatagggaccaatcaccaacagactttgaaagacacaacatgactggttactgatcatgacgagcttttattaccatggtgatttgtggactgaagagctagcccagaagtttgtactctatgcaaattacattaaatttactatggcacctaaattttgagacttgtgttgtgggactgatgttacttaactaaactatggtaagagaaatgcatgcatgtcatcaccaaagtgagaactgcctgtatttatctatgttacgcaaatggaatgtcagttatatcgtggggggttgggggtgggggaggattatctgtatcctcagttgccagaactgtgtctgccatataataggctgtcaatttttttaaatgagtgaatacatcattgaggtcattggtttctactaggtgttggatttcaaaagaaaacttctcttcatttgatcacttggcttgtgtgctgtattctcaagctctttctttaaagactactctctcttctttctgtttaaaacattccatcaaaaattcaacatcctctagaaagtaagaaagtgctaacaagaaaaaaaagtcaaccccaaaaccaaaacccacagtgactggagtacatcaaagaggcgcaggaaccaactgaaagagctcccaaaggccaaagctggaacactttcagcaaaaaaataagtaaagtagtaatgagttatagtccaaagtataaaataaatatctgagtccatactgatataaataaatgattgaataaattaatgggaaagaagagaagaatctccagtgcagaagagtcccagataatttatttatacactccactctaaaggaggtggaacataacttccactcctgaagtgtgggctgtgcactgcgacttctttgcaaagagtacagcatggaaaaaaggagaaaaaataactttacagtggaaaaatctgacaaatacagcttcagccaggtgatcaagagttcatcaacagttataaatcaacatcaagaatcataaatcatgtcactaatatgtacctttaatatgatgtgatgaagatgacacttttcctctattaccttccccccaaaactcacaaccccaatctaatcatgaggaaaacatcagacaaatttcaataaggaggcattctgcaacatcgggcccagaactcctcaaaactgtcaaggtcatcaaaatcaaggaaagtctaaaaaactgtcatagccaagagaagcttgaggagatatgacaagtaaatgtaacaggtatcctggatgggatgctggaagagaaaaacgacattaggtaaaaagaaaagatctaaataaactatgaactttgattaatca

SEQ ID NO: 6 - RNA, 1716 nt, Homo sapiens
  source 1..1716: mol_type = mRNA
agagcagggcctgacatctccccagaacagacgtttgaacagagcaggcttctgaggtctccaaaatgcctgtcccagcctcctggccccatcctcctggtcctttcctgcttctgactctactgctgggacttacagaagtggcaggtgaggaggagctacagatgattcagcctgagaagctcctgttggtcacagttggaaagacagccactctgcactgcactgtgacctccctgcttcccgtgggacccgtcctgtggttcagaggagttggaccaggccgggaattaatctacaatcaaaaagaaggccacttccccagggtaacaacagtttcagacctcacaaagagaaacaacatggacttttccatccgcatcagtagcatcaccccagcagatgtcggcacatactactgtgtgaagtttcgaaaagggagccctgagaacgtggagtttaagtctggaccaggcactgagatggctttgggtgccaaaccctctgcccccgtggtattgggccctgcggcgaggaccacacctgagcatacagtgagtttcacctgtgagtcccatggcttctctcccagagacatcaccctgaaatggttcaaaaatgggaatgagctctcagacttccagaccaacgtggaccccacaggacagagtgtggcctacagcatccgcagcacagccagggtggtactggacccctgggacgttcgctctcaggtcatctgcgaggtggcccatgtcaccttgcagggggaccctcttcgtgggactgccaacttgtctgaggccatccgagttccacccaccttggaggttactcaacagcccatgagggtggggaaccaggtaaacgtcacctgccaggtgaggaagttctacccccagagcctacagctgacctggtcggagaatggaaacgtgtgccagagagaaacagcctcgacccttacagagaacaaggatggtacctacaactggacaagctggttcctggtgaacatatctgaccaaagggatgatgtggtcctcacctgccaggtgaagcatgatgggcagctggcggtcagcaaacgccttgccctagaggtcacagtccaccagaaggaccagagctcagatgctacccctggcccggcatcatcccttactgcgctgctcctcatagctgtcctcctgggccccatctacgtcccctggaagcagaagacctgactctccttccttcctcccctgccacgtgggaccctcatctctgctgcctccttcctttcctgagaggctcagcttgagagaatgagccagtgagaagcttctctagacttggctccaaacatctcccctcccaagacatctgcctgcccacaggctcctgttgctccttcacacagacctggatgccccagagcaaggtcttcattcatggtcctgagcaggtgccatgggattgggctctgggcactgacttaacggcacctccctagaaggcgagaaacatgccaaatctaaacacaccaggactcccatccatcgccttgagactgaccgtaaaccacagacgctctccaggttctcaagagttatcctgccttccagattcctgcctatcccaactccccagccttgttgaggttctctattgcctcttgaatacaaatgcactcccaaagtggttttaagaaaataaaaagattatccttcc

SEQ ID NO: 7 - RNA, 4580 nt, Homo sapiens
  source 1..4580: mol_type = mRNA
actcacggccgctctccctcctcgctccgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgagtccttccggtgttcagtattcttgtctttgtgagtgcgtcccggggccgcctcggggcctgcctgccctcctgccaaagcctggaagaggattgaatggaccccagggtttggaaacaacctacagcatttgagcccctcacgtaggttttagagacgtacaatttttgtttgccctggctcagaaggagccggtgtaaggttgagataaaattccatatagacaactgagtttggatctcggctctgctgctttgtagctgtgggagttcaaacagcacctctttgagacttgggccccgcgtcggcacaatggggcaggaatagtccctgccagggtgactgtgtggattcaaggaggttggaagcactgagcccacgcttggcactgggtaggccatcactgaggcttgtgtctttccttcctgccctgaccctgcatagctgagtctcgtggcctctcaccactgatgggatctcggacacccggctcaccctcctcctggggcctcactttccttccccaccaaatgcagacatctaatgcttgtcctgtaatatccttgccagatgactgatgtcaagtgctggtttatagtgggtgctcaataaatgttagtttcctttctatccctccattagatcattaatgatattagtgctaattatgtaataaagttaaggcataaatatgttaa

SEQ ID NO: 8 - RNA, 4213 nt, Homo sapiens
  source 1..4213: mol_type = mRNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggtacagtccttggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 9 - RNA, 4603 nt, Homo sapiens
  source 1..4603: mol_type = mRNA
gcagccctcgctccctcccccggggcgcgcaggcgggcgctcgctcgcagagaagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgagtccttccggtgttcagtattcttgtctttgtgagtgcgtcccggggccgcctcggggcctgcctgccctcctgccaaagcctggaagaggattgaatggaccccagggtttggaaacaacctacagcatttgagcccctcacgtaggttttagagacgtacaatttttgtttgccctggctcagaaggagccggtgtaaggttgagataaaattccatatagacaactgagtttggatctcggctctgctgctttgtagctgtgggagttcaaacagcacctctttgagacttgggccccgcgtcggcacaatggggcaggaatagtccctgccagggtgactgtgtggattcaaggaggttggaagcactgagcccacgcttggcactgggtaggccatcactgaggcttgtgtctttccttcctgccctgaccctgcatagctgagtctcgtggcctctcaccactgatgggatctcggacacccggctcaccctcctcctggggcctcactttccttccccaccaaatgcagacatctaatgcttgtcctgtaatatccttgccagatgactgatgtcaagtgctggtttatagtgggtgctcaataaatgttagtttcctttctatccctccattagatcattaatgatattagtgctaattatgtaataaagttaaggcataaatatgttaa

SEQ ID NO: 10 - RNA, 3377 nt, Mus musculus
  source 1..3377: mol_type = mRNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 11 - RNA, 4043 nt, Mus musculus
  source 1..4043: mol_type = mRNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 12 - RNA, 3845 nt, Mus musculus
  source 1..3845: mol_type = mRNA
aagctcccctgccgcgggcagcctcttgcccactggagtctaaggactggccgggtgagaggccgagaccagggggcgatcggccgccacttccccagtccaccttaagaggaccaagtagccagcccgccgcgccgacctcagaaaaacaagtttgcgcaaagtggtgcgcggccagcctctgggcagagggagcggtgcttccaccgcctggcagccctgcgcgcggcggcgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 13 - RNA, 3389 nt, Mus musculus
  source 1..3389: mol_type = mRNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 14 - RNA, 3020 nt, Mus musculus
  source 1..3020: mol_type = mRNA
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacagataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 15 - RNA, 3820 nt, Mus musculus
  source 1..3820: mol_type = mRNA
aagctcccctgccgcgggcagcctcttgcccactggagtctaaggactggccgggtgagaggccgagaccagggggcgatcggccgccacttccccagtccaccttaagaggaccaagtagccagcccgccgcgccgacctcagaaaaacaagtttgcgcaaagtggtgcgcggccagcctctgggcagagggagcggtgcttccaccgcctggcagccctgcgcgcggcggcgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataa

SEQ ID NO: 16 - RNA, 4065 nt, Felis catus
  source 1..4065: mol_type = mRNA
tccaccttaagaggacgatgtagccagctcgcggcgctgaccttagaaaaacaagtttgcgcaaagtggagcgggggccccggcctctgggcagccccggcggcgctcccaccgcctcccagccgtcgccggcggcgcagccgcggcccatggagcccgccggcccggcccccggccgcctcgggccgctgctctgcctgctgctcgccgcgtcctgcgtctggacaggtgtggcaggtgaggcggaactgcaggtgatccagcccgagaagtcggtgtctgtggcggccggacagacggccactcttcactgcaccctgacctccctggtccccgttgggaaggtcgagtggttcaggggcacagggccaggccgggagttaatcttcagtttcagaggagaccctcactcccctcgagtaacaaatgtttcagacgccacaaggagaaacaacatggacttttccatccgcatcagtaacatcaccccagaagacacgggaacctactactgtgtgaagttccagaaagggaaccccgatgtggagtttaagtctggaccaggcacccaggtcaccgtgagcgccaaaccctctccccccgtggtgtcgggccccacggccagggccacacctgagcagactgtgagcttcacctgcgagtcccacggcttctcccccagaaacgtcaccctgaaatggttcaaaaacgggaatgagctggcagcctcccagaccaccgtggacccagagggagacagcgcttcctacagcatctccagcacaaccacgctgccgctggccccgggggacgttcgctcccaggtcatctgcgaggtggcccacgtgaccctgcaggggggccctcctcttcgtgggactgccaacttgtccgagaccctccgagttccacccaccttggaggttgcccagcaacccgtgacaggggaccaggtgaaggtcatttgccaagtgaagaagttctacccccagcgcctacagctgacctggttggagaacggaaacgtgtcccgaacagaaacggcctcgacggcctcgacggcctcgacggcctcgaacctcatagagaacaaggatgggacctttaactggacgagctggctcctggtgaatttatccgcccacagggaagatgtggttttcacctgccaggtgcagcatgacgggcagcccgcggtcaccaaaaaccatacccttgtggtctctgcccaccagaaggaccaggaaacgcatacgacccaggacaaaaatgaaagccagaccatcttcattgtggtgggcgtggtttgtgccctgctggtgactctcgtgatcgcagccctctacctcctccgaatccgacagaagaaagccaagggctccacttcttctacgaggttgcatgagcccgagaagaataccagagaaataacccagatccaggacaacaatgacatcacatacgcagacttgaacctgcccaaggggaagaaggccgccccgcgggccgccgagcccaacaaccacacagagtacgccagcattcagaccggcccgccgcccgcgcccgaggacaccctcacctacgccgacctagacatggtccacctcaaccgggcccccaagcagccagcccccaagcccgagccatcctactcggagtatgccagcgtccaggtccagaggaagtgaatgggaccgtggtcgtctctagggtctgtccccacgagccttcctgtcccgtgtgggacagccgtgatggggacagccagcccagttcccagagggccgggatggtgcaggccctggaactccaggatgaggctggttcttgtctccccaacccacgtggctcttcgacatttccagggcagccacagtccccccacatcaccacacgcggaggccgatgttgccagactagccgggggacccacctgggaactggccagaaccaccagggtccaagaaccctcatgcctgtctccatcaacatgcgggtctcggtcacggtcttggagaccctgattgccccccttcgtgctctgcagcctgatcttccagggtgaggagggagacaaccccacctccttttaccaccacccacctggggcttggggaagattgtccctttcgatcgaactgccccgtccgtggagaagctggaaaaaaaaccctccgggaccctcatcccaagactcggtgagactgctgccgagagtcctagcctccccatcccgggattgatgaggcgcaagccccgggggaggggaggagcctccgaggaacccccacacagccgccaccgccaccaccaccaaggactgagtaacaaaacccccctcctcctggtggccctccgagactccttgaggacccgccgtcccaaccgtgacgctccacacggcccctcctctcctttctagacccgagcttgcttcctccagctagcactaactcagcaacatctcgccgcggacgcctgtaaattatcgagaaatgtgaaatgtgcaatcttgaacctgaggtgttagaaaatttgatctgtggtgtttcgttttgttttggtttttttcttaaaacgacagcaacgttctcttggctctttgtcatgtgtcgaagtacctggtcgggtcttgtgcggtctaaggtttaccagttggtcgtcccgggggaaccctcttacggcggaaactgggttcccccacgtcccagaccccgaggatggggaggaaggttgcagtgcggctgccccgtggagacgcttagccttccctccagggcaaaataggtcacgttctcagccgagccacgtggctggtactgcggtcttctgtgcccccatcgtgctaagtccagacccgctcagtgctcctcctcctggctgcgtccttcacagtgcaagcttgtcgagacccgaggcctccgttttctcatccagaaagaggggagagaaaagatgcccccctgctccctgacatcttaccagcgctggaaggccacacgggggagggaagctatgcctgtctgttttccccgccccttaacgacaaccccattccagctctctgagctggtccctgcaggaggaatcttctacttctgctctcaaaccctttggggatcaaactggaataaatcccaaacagccaggaggactgggcagctgtgaagccaggactgaccctccttctgtccctctgtctcaggaggttgggcagatgctgtgacctgttgcctctgatccccacccttccagtcgggggcctggctgggcccagggccagcagacgttgcgagccctgtcgattcggtcttcacgcgtaaccaaccaagtcttagagcggaaaggttaatcttcgtcgccccctgggtccggccatccatcggatgccccagggattccaggccatcagaacaagaagctgagccatgctgcccatcagcaggtcctacgcatggaaccctgggagctcagctcagacccagttctttggggacagagatttggcatttcacacgacggcatccaggaatcgggtgagccaacgggccatgtggacagttttggccgatgagccagggtttctctgtggcatctgggagcacagtgacccagccacctggctcgggccatttccaagttccaaaaggttggcttgtcaaccttagcctccctctctcctgcccagagaaggcacatgtgcttgagtgtgtcccatgtacgtgcacgcgcacccacgtaggcacacagaattttaagagcgtgttctctcggtgccattttattttattttattttgaattttggaaggggaggaagggaatgaggccaaggaagttgtgcagctttagctccggcctggcagcctggagattcccataccttgtgtatcgaaccccaggaaaaggaagaggtcgaaacaacactacggaaggagcgtggtttcgggagtttattttaagactgctaggaaggaaacaggccccattttgtatatagttgcaacttaaactttttggcttgcaaaatatttttgtaataaagatttctgggtaacaacga

SEQ ID NO: 17 - RNA, 4059 nt, Canis lupus
  source 1..4059: mol_type = mRNA
ctctgggcagcccccgcggcgttcgccctgcgtcccagcccccgcggcggcgcagccgcggcccatggagcccgccggcccggcccccggccgcctcgggccgctgctctgcctcctgttcgccgcgtcctgcgcctggacaggtgtggcaggtgaggcagagctgcaggtgatccagcctgagaagtcagtgtctgtcgcggctggagagacagccactctgcgctgcaccctgacctccctgatccccgttgggaacgttgtgtggttcagggggacagggccaggccgggagatgatcttccatttcaaaggaggccacttcccccgagtaacaaatgtttcagactctacaaagaggaacaacacggatttttccatccgcatcagtaacatcaccccagcagacacgggaacctactactgtgtgaagttccagaaaggaaaccccgatgtggagttgaagtctggaccaggcaccctggtcactgtgagcgccaaaccctctccccccgtggtgtccggccccacggccagggccacgcctcagcagacagtgaacttcacctgcaagtcgcacggcttctctcccagaaacatcaccctgagatggttcaaaaacgggaatgaactgacagcctctcagaccactgtgtacccagaggaagacaacgcttcctacagcatctctagcacaaccaagctggtgctggccccgggggacgttcgctcccaggtcatctgcgaggtggcccacgtgaccctgcaggggggccctcctcttcgtgggactgccaacttgtctgagacccttcgagttccgcccaccttggaggtttcccagcaccccatggcaggggaccaggtgaatgtcacttgccaagtgaagaagttctaccctcagcgcctacagctgacctggttggagaatggaaacgtgtcccgaacagaaactccctcagtggcctccaacctcctagagaacaaggatgggacctttaactggacgagctggctcctggtgaattcatccacccacagggaagatgtggtgttcacctgccaggtgcagcatgacgggcagcctgcagtcaccaaaaaccatacccttgtggcctctgcccgccagaaggaccaggaaacacttaaacccgaggacaataatgacagccggagtatcttcattgtggtgggcgtggtttgtgccttgctggtggctctgctgatcgcagccctctacctcctccgaatccgacagaagaaagccaagggctccacttcttctacaaggttgcatgagcccgagaagaacaccagagaaataacccaggtacagtccttgatccaggacaacaatgacatcacgtatgcagacttgaacctgcccaaggggaagaagtctgccccccgggccgccgagcccaacaaccacacagagtatgccagcattcagactggcccgccgcccgcgcccgaggacaccctcacctacgccgacctggacatggtccacctcaaccgggctcccaagcagacggcccccaagcccgagccatcctactcggagtacgccagcgtccaggtccagaggaagtgaataggaccatggtcgtctctagggtctgtcctcatgagccttcctgtcccatgtgggacagccgtgatggggacagccagcccagttcccagagggccgggatggtgcaggccctgggaccccaggatgaggctggctcttgtttccccgacccgcctggctctccagcatttccagggcagccacagtccccccacatggccacacatggaggccaatgttgccagacccgccgggggaccgacctgggaactggccagagctgctgggggtccgggaatcctcatgcctgtctccatcaacatgtgggtcttggtcatggtcttggagaacctgacctcgcccttaatgctctgcagcctgatcttccgggatgaggagggagagaaccccacctcctttcactaccacccaactgaggcttggggaaaattttccctgtagatcgaactgccccatccatggagaagctggaaaaaaaatttaaattaaaaaaaaacctctgggacccgcatcccaagacttggtgagattgctgccaatagtcctagcctcccgatcccaggactaatgaggcacaagccctgggggaggggaggatcctccaaggaactcccacacaaccaccaccaccaccaccaccaaggactgagtaacaaagcccttctcctcctggtggccctccgagactccttgaggacccatcatcccaaccgtgaagctccacgtggcccctcctctcctttctagacctgagcttgcttcctccagctcgcactaactcagcaacatctcgctgtggacgcctgtaaattattgagaaatgtgaaatgtgcaatcttgaaactgaggtgttagaaaatttgatctgtggtgttttgttttgtttttttttttcttaaaatgacagcaacgttatcttggctctttgtcatgtgttgaagtacctggttgggtcttgtgcggtctaaggtttaccagttgattgtcctggagggatcttcttacagcagaaaccgagtccttccccgtcccagaccctgaggatggggcaaggaaggtcgtggtgtggctgctccatggaggtgcttagccttctttctccgggacaaaactggccacgtcctcagctgagccacgtggctgaaactgctgccttccgtgcccccagtgtgctaagtccaagcctgcccactacttctccaggctgcatccttcacagggcagtcttgtcgaaaactgaggcctcagtcttctcatccagaaagaggagaaagatgcccccatcccaccccacccccgctctctgccatctcacaagtgctggatgccgtgcaggggagggaagctatgcctggctgttgttcccaccctttagtgactgctcccattccagctcactgagctgatccctgtaggaggaatcttctgtttctgctctcatatcctttcgagatcaaactggaataaatccaagacaggattttgggcaggaggttgggcagatgctgcgacctcttacccctgacgccctcatatcctttcgagatcaaactggaataaatccaagacaggattttgggcaggaggttgggcagatgctgcgacctcttaccctgacgcccacacttccaatggggtgaagggggcctggctggtcccagggccagcatgtgttgcacgccctgtttattcagtcttcacgtatcaccgacctactgttggagctgagaggttcatattctctgcccccctgggtcctgccatttgatgtcccagggactccaggccatcagagcgagaggctgagccatcttgtccatcagcaggtcctacgcatggaacaccgggagctctgctcagacccagttctctggggacggtgatttggtgttttacatgatggcatccaggaattggctgagccaacaggccatgtgggccgctttggcccagtgagccagtttctctgtggcatctgggagcacagcggtgcagccacctggctcaagctatttccaaattccaaaaggatggcttgtaaaccttggtctccctctcttctgcccagagaaggcacatgtgcttgagcgtgcacacgtgtgcacacatgcacacacacacatacacacacacacacacaaacggaattttaagagaatgttttcttggtgccattttaatttaattttattttgaattttggaaggggagtaagggaatgaggccaaggaagttgtgcagcttttagctccggcctggcagcctggagattcccataccttgtgtatcgaaccccaggaaaaggaagaggtcgaaacaacactgcggaaggagcgtggtttcgggagtttattttaagactgctaggaaggaaacaggccccattttgtatatagttgcaacttaaactttttggcttgcaaaatatttttgtaataaagatttctgggtaacaacaa

SEQ ID NO: 18 - RNA, 19 nt, synthetic construct
  misc_feature 1..19: note = Target ID 1
  source 1..19: mol_type = other RNA
caaccgttacagagaacaa

SEQ ID NO: 19 - RNA, 19 nt, synthetic construct
  misc_feature 1..19: note = Target ID 1
  source 1..19: mol_type = other RNA
ttgttctctgtaacggttg

SEQ ID NO: 20 - RNA, 19 nt, synthetic construct
  misc_feature 1..19: note = Target ID 2
  source 1..19: mol_type = other RNA
gctgagaacactggatcta

SEQ ID NO: 21 - RNA, 19 nt, synthetic construct
  misc_feature 1..19: note = Target ID 2
  source 1..19: mol_type = other RNA
tagatccagtgttctcagc

SEQ ID NO: 22 - RNA, 19 nt, synthetic construct
  misc_feature 1..19: note = Target ID 3
  source 1..19: mol_type = other RNA
tgagaacactggatctaat

SEQ ID NO: 23 - RNA, 19 nt, synthetic construct
  misc_feature 1..19: note = Target ID 3
  source 1..19: mol_type = other RNA
attagatccagtgttctca

SEQ ID NO: 24 - RNA, 19 nt, synthetic construct
  misc_feature 1..19: note = Target ID 4
  source 1..19: mol_type = other RNA
ggatctaatgaacggaaca

SEQ ID NO: 25 - RNA, 19 nt, synthetic construct
  misc_feature 1..19: note = Target ID 4
  source 1..19: mol_type = other RNA
tgttccgttcattagatcc

SEQ ID NO: 26 - RNA, 19 nt, synthetic construct
  misc_feature 1..19: note = Target ID 5
  source 1..19: mol_type = other RNA
cggagtatgccagcattca

SEQ ID NO: 27 - RNA, 19 nt, synthetic construct
  misc_feature 1..19: note = Target ID 5
  source 1..19: mol_type = other RNA
tgaatgctggcatactccg

SEQ ID NO: 28 - RNA, 19 nt, synthetic construct
  misc_feature 1..19: note = Target ID 6
  source 1..19: mol_type = other RNA
ctaatgaacggaacatcta

SEQ ID NO: 29 - RNA, 19 nt, synthetic construct
  misc_feature 1..19: note = Target ID 6
  source 1..19: mol_type = other RNA
tagatgttccgttcattag

SEQ ID NO: 30 - RNA, 19 nt, synthetic construct
  misc_feature 1..19: note = Target ID 7
  source 1..19: mol_type = other RNA
ccctctacctcgtccgaat

SEQ ID NO: 31 - RNA, 19 nt, synthetic construct
  misc_feature 1..19: note = Target ID 7
  source 1..19: mol_type = other RNA
attcggacgaggtagaggg

SEQ ID NO: 32 - RNA, 19 nt, synthetic construct
  misc_feature 1..19: note = Target ID 8
  source 1..19: mol_type = other RNA
ggatctaatgaacggaaca

SEQ ID NO: 33 - RNA, 19 nt, synthetic construct
  misc_feature 1..19: note = Target ID 8
  source 1..19: mol_type = other RNA
tgttccgttcattagatcc

SEQ ID NO: 34 - RNA, 19 nt, synthetic construct
  misc_feature 1..19: note = Target ID 9
  source 1..19: mol_type = other RNA
gatctaatgaacggaacat

SEQ ID NO: 35 - RNA, 19 nt, synthetic construct
  misc_feature 1..19: note = Target ID 9
  source 1..19: mol_type = other RNA
atgttccgttcattagatc

SEQ ID NO: 36 - RNA, 19 nt, synthetic construct
  misc_feature 1..19: note = Target ID 10
  source 1..19: mol_type = other RNA
ggaggagaaaatcccacct

SEQ ID NO: 37 - RNA, 19 nt, synthetic construct
  misc_feature 1..19: note = Target ID 10
  source 1..19: mol_type = other RNA
aggtgggattttctcctcc

SEQ ID NO: 38 - RNA, 19 nt, synthetic construct
  misc_feature 1..19: note = Target ID 11
  source 1..19: mol_type = other RNA
cgtgatcttggctgtctgt

SEQ ID NO: 39 - RNA, 19 nt, synthetic construct
  misc_feature 1..19: note = Target ID 11
  source 1..19: mol_type = other RNA
acagacagccaagatcacg

SEQ ID NO: 40 - RNA, 19 nt, synthetic construct
  misc_feature 1..19: note = Target ID 12
  source 1..19: mol_type = other RNA
gaagaatgccagagaaata

SEQ ID NO: 41 - RNA, 19 nt, synthetic construct
  misc_feature 1..19: note = Target ID 12
  source 1..19: mol_type = other RNA
tatttctctggcattcttc

SEQ ID NO: 42 - RNA, 19 nt, synthetic construct
  misc_feature 1..19: note = Target ID 13
  source 1..19: mol_type = other RNA
gagaagaatgccagagaaa

SEQ ID NO: 43 - RNA, 19 nt, synthetic construct
  misc_feature 1..19: note = Target ID 13
  source 1..19: mol_type = other RNA
tttctctggcattcttctc

SEQ ID NO: 44 - RNA, 19 nt, synthetic construct
  misc_feature 1..19: note = Target ID 14
  source 1..19: mol_type = other RNA
ccgatgacgtggagtttaa

SEQ ID NO: 45 - RNA, 19 nt, synthetic construct
  misc_feature 1..19: note = Target ID 14
  source 1..19: mol_type = other RNA
ttaaactccacgtcatcgg

SEQ ID NO: 46 - RNA, 19 nt, synthetic construct
  misc_feature 1..19: note = Target ID 15
  source 1..19: mol_type = other RNA
ggttgcagctggagagaca

SEQ ID NO: 47 - RNA, 19 nt, synthetic construct
  misc_feature 1..19: note = Target ID 15
  source 1..19: mol_type = other RNA
tgtctctccagctgcaacc

SEQ ID NO: 48 - RNA, 19 nt, synthetic construct
  misc_feature 1..19: note = Target ID 16
  source 1..19: mol_type = other RNA
cgagaagaatgccagagaa

SEQ ID NO: 49 - RNA, 19 nt, synthetic construct
  misc_feature 1..19: note = Target ID 16
  source 1..19: mol_type = other RNA
ttctctggcattcttctcg

SEQ ID NO: 50 - RNA, 19 nt, synthetic construct
  misc_feature 1..19: note = Target ID 17
  source 1..19: mol_type = other RNA
tgcatgagcccgagaagaa

SEQ ID NO: 51 - RNA, 19 nt, synthetic construct
  misc_feature 1..19: note = Target ID 17
  source 1..19: mol_type = other RNA
ttcttctcgggctcatgca

SEQ ID NO: 52 - RNA, 19 nt, synthetic construct
  misc_feature 1..19: note = Target ID 18
  source 1..19: mol_type = other RNA
ggacacaaatgatatcaca

SEQ ID NO: 53 - RNA, 19 nt, synthetic construct
  misc_feature 1..19: note = Target ID 18
  source 1..19: mol_type = other RNA
tgtgatatcatttgtgtcc

SEQ ID NO: 54 - RNA, 19 nt, synthetic construct
  misc_feature 1..19: note = Target ID 19
  source 1..19: mol_type = other RNA
gcgaggacgttcactctca

SEQ ID NO: 55 - RNA, 19 nt, synthetic construct
  misc_feature 1..19: note = Target ID 19
  source 1..19: mol_type = other RNA
tgagagtgaacgtcctcgc

SEQ ID NO: 56 - RNA, 19 nt, synthetic construct
  misc_feature 1..19: note = Target ID 20
  source 1..19: mol_type = other RNA
aggaggagctgcaggtgat

SEQ ID NO: 57 - RNA, 19 nt, synthetic construct
  misc_feature 1..19: note = Target ID 20
  source 1..19: mol_type = other RNA
atcacctgcagctcctcct

SEQ ID NO: 58 - RNA, 19 nt, synthetic construct
  misc_feature 1..19: note = Target ID 21
  source 1..19: mol_type = other RNA
ccaacaaccacacggagta

SEQ ID NO: 59 - RNA, 19 nt, synthetic construct
  misc_feature 1..19: note = Target ID 21
  source 1..19: mol_type = other RNA
tactccgtgtggttgttgg

SEQ ID NO: 60 - RNA, 19 nt, synthetic construct
  misc_feature 1..19: note = Target ID 22
  source 1..19: mol_type = other RNA
tcacatatgcagacctgaa

SEQ ID NO: 61 - RNA, 19 nt, synthetic construct
  misc_feature 1..19: note = Target ID 22
  source 1..19: mol_type = other RNA
ttcaggtctgcatatgtga

SEQ ID NO: 62 - RNA, 19 nt, synthetic construct
  misc_feature 1..19: note = Target ID 23
  source 1..19: mol_type = other RNA
ggagagagcgtgtcctaca

SEQ ID NO: 63 - RNA, 19 nt, synthetic construct
  misc_feature 1..19: note = Target ID 23
  source 1..19: mol_type = other RNA
tgtaggacacgctctctcc

SEQ ID NO: 64 - RNA, 19 nt, synthetic construct
  misc_feature 1..19: note = Target ID 24
  source 1..19: mol_type = other RNA
ccgaatcagacagaagaaa

SEQ ID NO: 65 - RNA, 19 nt, synthetic construct
  misc_feature 1..19: note = Target ID 24
  source 1..19: mol_type = other RNA
tttcttctgtctgattcgg

SEQ ID NO: 66 - RNA, 19 nt, synthetic construct
  misc_feature 1..19: note = Target ID 25
  source 1..19: mol_type = other RNA
gaggaggagctgcaggtga

SEQ ID NO: 67 - RNA, 19 nt, synthetic construct
  misc_feature 1..19: note = Target ID 25
  source 1..19: mol_type = other RNA
tcacctgcagctcctcctc

SEQ ID NO: 68 - RNA, 19 nt, synthetic construct
  misc_feature 1..19: note = Target ID 26
  source 1..19: mol_type = other RNA
cctcaaccgttacagagaa

SEQ ID NO: 69 - RNA, 19 nt, synthetic construct
  misc_feature 1..19: note = Target ID 26
  source 1..19: mol_type = other RNA
ttctctgtaacggttgagg

SEQ ID NO: 70 - RNA, 19 nt, synthetic construct
  misc_feature 1..19: note = Target ID 27
  source 1..19: mol_type = other RNA
gaacactggatctaatgaa

SEQ ID NO: 71 - RNA, 19 nt, synthetic construct
  misc_feature 1..19: note = Target ID 27
  source 1..19: mol_type = other RNA
ttcattagatccagtgttc

SEQ ID NO: 72 - RNA, 19 nt, synthetic construct
  misc_feature 1..19: note = Target ID 28
  source 1..19: mol_type = other RNA
tctatattgtggtgggtgt

SEQ ID NO: 73 - RNA, 19 nt, synthetic construct
  misc_feature 1..19: note = Target ID 28
  source 1..19: mol_type = other RNA
acacccaccacaatataga

SEQ ID NO: 74 - RNA, 19 nt, synthetic construct
  misc_feature 1..19: note = Target ID 29
  source 1..19: mol_type = other RNA
cgaatcagacagaagaaag

SEQ ID NO: 75 - RNA, 19 nt, synthetic construct
  misc_feature 1..19: note = Target ID 29
  source 1..19: mol_type = other RNA
ctttcttctgtctgattcg

SEQ ID NO: 76 - RNA, 19 nt, synthetic construct
  misc_feature 1..19: note = Luciferase
  source 1..19: mol_type = other RNA
cttacgctgagtacttcga

SEQ ID NO: 77 - RNA, 19 nt, synthetic construct
  misc_feature 1..19: note = Luciferase
  source 1..19: mol_type = other RNA
tcgaagtactcagcgtaag

SEQ ID NO: 78 - RNA, 19 nt, synthetic construct
  misc_feature 1..19: note = Target ID_m1
  source 1..19: mol_type = other RNA
ccggatcaaacagaagaaa

SEQ ID NO: 79 - RNA, 19 nt, synthetic construct
  misc_feature 1..19: note = Target ID_m1
  source 1..19: mol_type = other RNA
tttcttctgtttgatccgg

SEQ ID NO: 80 - RNA, 19 nt, synthetic construct
  misc_feature 1..19: note = Target ID_m2
  source 1..19: mol_type = other RNA
gaagaacgccagggaaata

SEQ ID NO: 81 - RNA, 19 nt, synthetic construct
  misc_feature 1..19: note = Target ID_m2
  source 1..19: mol_type = other RNA
tatttccctggcgttcttc

SEQ ID NO: 82 - RNA, 19 nt, synthetic construct
  misc_feature 1..19: note = Target ID_m3
  source 1..19: mol_type = other RNA
agacctgaatctgcccaaa

SEQ ID NO: 83 - RNA, 19 nt, synthetic construct
  misc_feature 1..19: note = Target ID_m3
  source 1..19: mol_type = other RNA
tttgggcagattcaggtct

SEQ ID NO: 84 - RNA, 19 nt, synthetic construct
  misc_feature 1..19: note = Target ID_m4
  source 1..19: mol_type = other RNA
gcctaacaaccacacagaa

SEQ ID NO: 85 - RNA, 19 nt, synthetic construct
  misc_feature 1..19: note = Target ID_m4
  source 1..19: mol_type = other RNA
ttctgtgtggttgttaggc

SEQ ID NO: 86 - RNA, 19 nt, synthetic construct
  misc_feature 1..19: note = Target ID_m5
  source 1..19: mol_type = other RNA
gtgcctaggccagaggata

SEQ ID NO: 87 - RNA, 19 nt, synthetic construct
  misc_feature 1..19: note = Target ID_m5
  source 1..19: mol_type = other RNA
tatcctctggcctaggcac

SEQ ID NO: 88 - RNA, 19 nt, synthetic construct
  misc_feature 1..19: note = Target ID_m6
  source 1..19: mol_type = other RNA
aagcctgagccatctttct

SEQ ID NO: 89 - RNA, 19 nt, synthetic construct
  misc_feature 1..19: note = Target ID_m6
  source 1..19: mol_type = other RNA
agaaagatggctcaggctt

SEQ ID NO: 90 - RNA, 19 nt, synthetic construct
  misc_feature 1..19: note = Target ID_m7
  source 1..19: mol_type = other RNA
gccatctttctcagagtat

SEQ ID NO: 91 - RNA, 19 nt, synthetic construct
  misc_feature 1..19: note = Target ID_m7
  source 1..19: mol_type = other RNA
atactctgagaaagatggc

SEQ ID NO: 92 - RNA, 19 nt, synthetic construct
  misc_feature 1..19: note = Target ID_m8
  source 1..19: mol_type = other RNA
tccggatcaaacagaagaa

SEQ ID NO: 93 - RNA, 19 nt, synthetic construct
  misc_feature 1..19: note = Target ID_m8
  source 1..19: mol_type = other RNA
ttcttctgtttgatccgga

SEQ ID NO: 94 - RNA, 19 nt, synthetic construct
  misc_feature 1..19: note = Target ID_m9
  source 1..19: mol_type = other RNA
aagaacgccagggaaataa

SEQ ID NO: 95 - RNA, 19 nt, synthetic construct
  misc_feature 1..19: note = Target ID_m9
  source 1..19: mol_type = other RNA
ttatttccctggcgttctt

SEQ ID NO: 96 - RNA, 19 nt, synthetic construct
  misc_feature 1..19: note = Target ID_m10
  source 1..19: mol_type = other RNA
ccatctttctcagagtatg

SEQ ID NO: 97 - RNA, 19 nt, synthetic construct
  misc_feature 1..19: note = Target ID_m10
  source 1..19: mol_type = other RNA
catactctgagaaagatgg

SEQ ID NO: 98 - RNA, 19 nt, synthetic construct
  misc_feature 1..19: note = Target ID_m11
  source 1..19: mol_type = other RNA
acatcaacgacatcacata

SEQ ID NO: 99 - RNA, 19 nt, synthetic construct
  misc_feature 1..19: note = Target ID_m11
  source 1..19: mol_type = other RNA
tatgtgatgtcgttgatgt

SEQ ID NO: 100 - RNA, 19 nt, synthetic construct
  misc_feature 1..19: note = Target ID_m12
  source 1..19: mol_type = other RNA
agaggataccctcacctat

SEQ ID NO: 101 - RNA, 19 nt, synthetic construct
  misc_feature 1..19: note = Target ID_m12
  source 1..19: mol_type = other RNA
ataggtgagggtatcctct

SEQ ID NO: 102 - RNA, 19 nt, synthetic construct
  misc_feature 1..19: note = Target ID_m13
  source 1..19: mol_type = other RNA
tcacatacgcagacctgaa

SEQ ID NO: 103 - RNA, 19 nt, synthetic construct
  misc_feature 1..19: note = Target ID_m13
  source 1..19: mol_type = other RNA
ttcaggtctgcgtatgtga

SEQ ID NO: 104 - RNA, 19 nt, synthetic construct
  misc_feature 1..19: note = Target ID_m14
  source 1..19: mol_type = other RNA
gaaccaggccctgttctta

SEQ ID NO: 105 - RNA, 19 nt, synthetic construct
  misc_feature 1..19: note = Target ID_m14
  source 1..19: mol_type = other RNA
taagaacagggcctggttc

SEQ ID NO: 106 - RNA, 19 nt, synthetic construct
  misc_feature 1..19: note = Target ID_m15
  source 1..19: mol_type = other RNA
ctctacccaacttgagctt

SEQ ID NO: 107 - RNA, 19 nt, synthetic construct
  misc_feature 1..19: note = Target ID_m15
  source 1..19: mol_type = other RNA
aagctcaagttgggtagag

SEQ ID NO: 108 - RNA, 19 nt, synthetic construct
  misc_feature 1..19: note = Target ID_m16
  source 1..19: mol_type = other RNA
ctggaatccaagaagtgtt

SEQ ID NO: 109 - RNA, 19 nt, synthetic construct
  misc_feature 1..19: note = Target ID_m16
  source 1..19: mol_type = other RNA
aacacttcttggattccag

SEQ ID NO: 110 - RNA, 19 nt, synthetic construct
  misc_feature 1..19: note = Target ID_m17
  source 1..19: mol_type = other RNA
gagaagaacgccagggaaa

SEQ ID NO: 111 - RNA, 19 nt, synthetic construct
  misc_feature 1..19: note = Target ID_m17
  source 1..19: mol_type = other RNA
tttccctggcgttcttctc

SEQ ID NO: 112 - RNA, 19 nt, synthetic construct
  misc_feature 1..19: note = Target ID_m18
  source 1..19: mol_type = other RNA
cctaacaaccacacagaat

SEQ ID NO: 113 - RNA, 19 nt, synthetic construct
  misc_feature 1..19: note = Target ID_m18
  source 1..19: mol_type = other RNA
attctgtgtggttgttagg

SEQ ID NO: 114 - RNA, 19 nt, synthetic construct
  misc_feature 1..19: note = Target ID_m19
  source 1..19: mol_type = other RNA
gtgtccaggtccagaggaa

SEQ ID NO: 115 - RNA, 19 nt, synthetic construct
  misc_feature 1..19: note = Target ID_m19
  source 1..19: mol_type = other RNA
ttcctctggacctggacac

SEQ ID NO: 116 - RNA, 19 nt, synthetic construct
  misc_feature 1..19: note = Target ID_m20
  source 1..19: mol_type = other RNA
aagcattgagacaggcaaa

SEQ ID NO: 117 - RNA, 19 nt, synthetic construct
  misc_feature 1..19: note = Target ID_m20
  source 1..19: mol_type = other RNA
tttgcctgtctcaatgctt

SEQ ID NO: 118 - RNA, 19 nt, synthetic construct
  misc_feature 1..19: note = Target ID_m21
  source 1..19: mol_type = other RNA
ctaacaaccacacagaata

SEQ ID NO: 119 - RNA, 19 nt, synthetic construct
  misc_feature 1..19: note = Target ID_m21
  source 1..19: mol_type = other RNA
tattctgtgtggttgttag

SEQ ID NO: 120 - RNA, 19 nt, synthetic construct
  misc_feature 1..19: note = Target ID_m22
  source 1..19: mol_type = other RNA
tgcacgagcccgagaagaa

SEQ ID NO: 121 - RNA, 19 nt, synthetic construct
  misc_feature 1..19: note = Target ID_m22
  source 1..19: mol_type = other RNA
ttcttctcgggctcgtgca

SEQ ID NO: 122 - RNA, 19 nt, synthetic construct
  misc_feature 1..19: note = Target ID_m23
  source 1..19: mol_type = other RNA
caaatgacatcaacgacat

SEQ ID NO: 123 - RNA, 19 nt, synthetic construct
  misc_feature 1..19: note = Target ID_m23
  source 1..19: mol_type = other RNA
atgtcgttgatgtcatttg

SEQ ID NO: 124 - RNA, 19 nt, synthetic construct
  misc_feature 1..19: note = Target ID_m24
  source 1..19: mol_type = other RNA
cgagaagaacgccagggaa

SEQ ID NO: 125 - RNA, 19 nt, synthetic construct
  misc_feature 1..19: note = Target ID_m24
  source 1..19: mol_type = other RNA
ttccctggcgttcttctcg

SEQ ID NO: 126 - RNA, 19 nt, synthetic construct
  misc_feature 1..19: note = Target ID_m25
  source 1..19: mol_type = other RNA
acaaatgacatcaacgaca

SEQ ID NO: 127 - RNA, 19 nt, synthetic construct
  misc_feature 1..19: note = Target ID_m25
  source 1..19: mol_type = other RNA
tgtcgttgatgtcatttgt

SEQ ID NO: 128 - RNA, 19 nt, synthetic construct
  misc_feature 1..19: note = Target ID_m26
  source 1..19: mol_type = other RNA
gataataatgctacccaca

SEQ ID NO: 129 - RNA, 19 nt, synthetic construct
  misc_feature 1..19: note = Target ID_m26
  source 1..19: mol_type = other RNA
tgtgggtagcattattatc

SEQ ID NO: 130 - RNA, 19 nt, synthetic construct
  misc_feature 1..19: note = Target ID_m27
  source 1..19: mol_type = other RNA
agaagaacgccagggaaat

SEQ ID NO: 131 - RNA, 19 nt, synthetic construct
  misc_feature 1..19: note = Target ID_m27
  source 1..19: mol_type = other RNA
atttccctggcgttcttct

SEQ ID NO: 132 - RNA, 19 nt, synthetic construct
  misc_feature 1..19: note = Target ID_m28
  source 1..19: mol_type = other RNA
taacaaccacacagaatat

SEQ ID NO: 133 - RNA, 19 nt, synthetic construct
  misc_feature 1..19: note = Target ID_m28
  source 1..19: mol_type = other RNA
atattctgtgtggttgtta

SEQ ID NO: 134 - RNA, 19 nt, synthetic construct
  misc_feature 1..19: note = Target ID_m29
  source 1..19: mol_type = other RNA
ggatcaaacagaagaaagc

SEQ ID NO: 135 - RNA, 19 nt, synthetic construct
  misc_feature 1..19: note = Target ID_m29
  source 1..19: mol_type = other RNA
gctttcttctgtttgatcc

SEQ ID NO: 136 - RNA, 19 nt, synthetic construct
  misc_feature 1..19: note = Target ID_m30
  source 1..19: mol_type = other RNA
tcaaacagaagaaagccaa

SEQ ID NO: 137 - RNA, 19 nt, synthetic construct
  misc_feature 1..19: note = Target ID_m30
  source 1..19: mol_type = other RNA
ttggctttcttctgtttga

SEQ ID NO: 138 - RNA, 19 nt, synthetic construct
  misc_feature 1..19: note = Target ID_m31
  source 1..19: mol_type = other RNA
acacaaatgacatcaacga

SEQ ID NO: 139 - RNA, 19 nt, synthetic construct
  misc_feature 1..19: note = Target ID_m31
  source 1..19: mol_type = other RNA
tcgttgatgtcatttgtgt

SEQ ID NO: 140 - RNA, 26 nt, synthetic construct
  misc_feature 1..26: note = sihSIRPa_1-PM
  source 1..26: mol_type = other RNA
mcaamcmcgmtmtamcagagaamcaa

SEQ ID NO: 141 - RNA, 20 nt, synthetic construct
  misc_feature 1..20: note = sihSIRPa_1-PM
  source 1..20: mol_type = other RNA
ttgttctctgmtaacggttg

SEQ ID NO: 142 - RNA, 27 nt, synthetic construct
  misc_feature 1..27: note = sihSIRPa_2-PM
  source 1..27: mol_type = other RNA
gmcmtgagaamcamcmtggamtmcmta

SEQ ID NO: 143 - RNA, 22 nt, synthetic construct
  misc_feature 1..22: note = sihSIRPa_2-PM
  source 1..22: mol_type = other RNA
mtagatcmcagtgttctmcagc

SEQ ID NO: 144 - RNA, 27 nt, synthetic construct
  misc_feature 1..27: note = sihSIRPa_3-PM
  source 1..27: mol_type = other RNA
mtgagaamcamcmtggamtmcmtaamt

SEQ ID NO: 145 - RNA, 22 nt, synthetic construct
  misc_feature 1..22: note = sihSIRPa_3-PM
  source 1..22: mol_type = other RNA
atmtagatcmcagtgttctmca

SEQ ID NO: 146 - RNA, 25 nt, synthetic construct
  misc_feature 1..25: note = sihSIRPa_4-PM
  source 1..25: mol_type = other RNA
ggamtmcmtaamtgaamcggaamca

SEQ ID NO: 147 - RNA, 21 nt, synthetic construct
  misc_feature 1..21: note = sihSIRPa_4-PM
  source 1..21: mol_type = other RNA
tgttccgttmcatmtagatcc

SEQ ID NO: 148 - RNA, 33 nt, synthetic construct
  misc_feature 1..33: note = sihSIRPa_7-PM
  source 1..33: mol_type = other RNA
mcmcmcmtmcmtamcmcmtmcgmtmcmcgaamt

SEQ ID NO: 149 - RNA, 20 nt, synthetic construct
  misc_feature 1..20: note = sihSIRPa_7-PM
  source 1..20: mol_type = other RNA
attcggacgaggmtagaggg

SEQ ID NO: 150 - RNA, 25 nt, synthetic construct
  misc_feature 1..25: note = sihSIRPa_8-PM
  source 1..25: mol_type = other RNA
ggamtmcmtaamtgaamcggaamca

SEQ ID NO: 151 - RNA, 21 nt, synthetic construct
  misc_feature 1..21: note = sihSIRPa_8-PM
  source 1..21: mol_type = other RNA
tgttccgttmcatmtagatcc

SEQ ID NO: 152 - RNA, 23 nt, synthetic construct
  misc_feature 1..23: note = sihSIRPa_12-PM
  source 1..23: mol_type = other RNA
gaagaamtgmcmcagagaaamta

SEQ ID NO: 153 - RNA, 21 nt, synthetic construct
  misc_feature 1..21: note = sihSIRPa_12-PM
  source 1..21: mol_type = other RNA
mtatttctctggmcattcttc

SEQ ID NO: 154 - RNA, 22 nt, synthetic construct
  misc_feature 1..22: note = sihSIRPa_13-PM
  source 1..22: mol_type = other RNA
gagaagaamtgmcmcagagaaa

SEQ ID NO: 155 - RNA, 20 nt, synthetic construct
  misc_feature 1..20: note = sihSIRPa_13-PM
  source 1..20: mol_type = other RNA
tttctctggmcattcttctc

SEQ ID NO: 156 - RNA, 23 nt, synthetic construct
  misc_feature 1..23: note = sihSIRPa_16-PM
  source 1..23: mol_type = other RNA
mcgagaagaamtgmcmcagagaa

SEQ ID NO: 157 - RNA, 20 nt, synthetic construct
  misc_feature 1..20: note = sihSIRPa_16-PM
  source 1..20: mol_type = other RNA
ttctctggmcattcttctcg

SEQ ID NO: 158 - RNA, 25 nt, synthetic construct
  misc_feature 1..25: note = sihSIRPa_17-PM
  source 1..25: mol_type = other RNA
mtgmcamtgagmcmcmcgagaagaa

SEQ ID NO: 159 - RNA, 21 nt, synthetic construct
  misc_feature 1..21: note = sihSIRPa_17-PM
  source 1..21: mol_type = other RNA
ttcttctcgggctmcatgmca

SEQ ID NO: 160 - RNA, 26 nt, synthetic construct
  misc_feature 1..26: note = sihSIRPa_18-PM
  source 1..26: mol_type = other RNA
ggamcamcaaamtgamtamtmcamca

SEQ ID NO: 161 - RNA, 21 nt, synthetic construct
  misc_feature 1..21: note = sihSIRPa_18-PM
  source 1..21: mol_type = other RNA
tgtgamtatmcatttgtgtcc

SEQ ID NO: 162 - RNA, 29 nt, synthetic construct
  misc_feature 1..29: note = sihSIRPa_19-PM
  source 1..29: mol_type = other RNA
gmcgaggamcgmtmtmcamcmtmcmtmca

SEQ ID NO: 163 - RNA, 19 nt, synthetic construct
  misc_feature 1..19: note = sihSIRPa_19-PM
  source 1..19: mol_type = other RNA
tgagagtgaacgtcctcgc

SEQ ID NO: 164 - RNA, 24 nt, synthetic construct
  misc_feature 1..24: note = sihSIRPa_20-PM
  source 1..24: mol_type = other RNA
aggaggagmcmtgmcaggmtgamt

SEQ ID NO: 165 - RNA, 21 nt, synthetic construct
  misc_feature 1..21: note = sihSIRPa_20-PM
  source 1..21: mol_type = other RNA
atmcacctgmcagctcctcct

SEQ ID NO: 166 - RNA, 27 nt, synthetic construct
  misc_feature 1..27: note = sihSIRPa_21-PM
  source 1..27: mol_type = other RNA
mcmcaamcaamcmcamcamcggagmta

SEQ ID NO: 167 - RNA, 20 nt, synthetic construct
  misc_feature 1..20: note = sihSIRPa_21-PM
  source 1..20: mol_type = other RNA
mtactccgtgtggttgttgg

SEQ ID NO: 168 - RNA, 28 nt, synthetic construct
  misc_feature 1..28: note = sihSIRPa_22-PM
  source 1..28: mol_type = other RNA
mtmcamcamtamtgmcagamcmcmtgaa

SEQ ID NO: 169 - RNA, 22 nt, synthetic construct
  misc_feature 1..22: note = sihSIRPa_22-PM
  source 1..22: mol_type = other RNA
ttmcaggtctgmcamtatgtga

SEQ ID NO: 170 - RNA, 26 nt, synthetic construct
  misc_feature 1..26: note = sihSIRPa_23-PM
  source 1..26: mol_type = other RNA
ggagagagmcgmtgmtmcmcmtamca

SEQ ID NO: 171 - RNA, 21 nt, synthetic construct
  misc_feature 1..21: note = sihSIRPa_23-PM
  source 1..21: mol_type = other RNA
tgmtaggamcacgctctctcc

SEQ ID NO: 172 - RNA, 24 nt, synthetic construct
  misc_feature 1..24: note = sihSIRPa_24-PM
  source 1..24: mol_type = other RNA
mcmcgaamtmcagamcagaagaaa

SEQ ID NO: 173 - RNA, 21 nt, synthetic construct
  misc_feature 1..21: note = sihSIRPa_24-PM
  source 1..21: mol_type = other RNA
tttcttcmtgtcmtgattcgg

SEQ ID NO: 174 - RNA, 23 nt, synthetic construct
  misc_feature 1..23: note = sihSIRPa_25-PM
  source 1..23: mol_type = other RNA
gaggaggagmcmtgmcaggmtga

SEQ ID NO: 175 - RNA, 21 nt, synthetic construct
  misc_feature 1..21: note = sihSIRPa_25-PM
  source 1..21: mol_type = other RNA
tmcacctgmcagctcctcctc

SEQ ID NO: 176 - RNA, 28 nt, synthetic construct
  misc_feature 1..28: note = sihSIRPa_26-PM
  source 1..28: mol_type = other RNA
mcmcmtmcaamcmcgmtmtamcagagaa

SEQ ID NO: 177 - RNA, 20 nt, synthetic construct
  misc_feature 1..20: note = sihSIRPa_26-PM
  source 1..20: mol_type = other RNA
ttctctgmtaacggttgagg

SEQ ID NO: 178 - RNA, 26 nt, synthetic construct
  misc_feature 1..26: note = sihSIRPa_27-PM
  source 1..26: mol_type = other RNA
gaamcamcmtggamtmcmtaamtgaa

SEQ ID NO: 179 - RNA, 22 nt, synthetic construct
  misc_feature 1..22: note = sihSIRPa_27-PM
  source 1..22: mol_type = other RNA
ttmcatmtagatcmcagtgttc

SEQ ID NO: 180 - RNA, 29 nt, synthetic construct
  misc_feature 1..29: note = sihSIRPa_28-PM
  source 1..29: mol_type = other RNA
mtmcmtamtamtmtgmtggmtgggmtgmt

SEQ ID NO: 181 - RNA, 25 nt, synthetic construct
  misc_feature 1..25: note = sihSIRPa_28-PM
  source 1..25: mol_type = other RNA
amcaccmcacmcamcaamtamtaga

SEQ ID NO: 182 - RNA, 23 nt, synthetic construct
  misc_feature 1..23: note = sihSIRPa_29-PM
  source 1..23: mol_type = other RNA
mcgaamtmcagamcagaagaaag

SEQ ID NO: 183 - RNA, 22 nt, synthetic construct
  misc_feature 1..22: note = sihSIRPa_29-PM
  source 1..22: mol_type = other RNA
ctttcttcmtgtcmtgatmtcg

SEQ ID NO: 184 - RNA, 30 nt, synthetic construct
  misc_feature 1..30: note = siLuc
  source 1..30: mol_type = other RNA
mcmtmtamcgmcmtgagmtamcmtmtmcga

SEQ ID NO: 185 - RNA, 22 nt, synthetic construct
  misc_feature 1..22: note = siLuc
  source 1..22: mol_type = other RNA
tcgaagmtactmcagcgmtaag

SEQ ID NO: 186 - RNA, 28 nt, synthetic construct
  misc_feature 1..28: note = simSIRPa_3-PM
  source 1..28: mol_type = other RNA
agamcmcmtgaamtmcmtgmcmcmcaaa

SEQ ID NO: 187 - RNA, 21 nt, synthetic construct
  misc_feature 1..21: note = simSIRPa_3-PM
  source 1..21: mol_type = other RNA
tttgggmcagattmcaggtct

SEQ ID NO: 188 - RNA, 26 nt, synthetic construct
  misc_feature 1..26: note = simSIRPa_5-PM
  source 1..26: mol_type = other RNA
gmtgmcmcmtaggmcmcagaggamta

SEQ ID NO: 189 - RNA, 22 nt, synthetic construct
  misc_feature 1..22: note = simSIRPa_5-PM
  source 1..22: mol_type = other RNA
mtatcctctggccmtaggmcac

SEQ ID NO: 190 - RNA, 31 nt, synthetic construct
  misc_feature 1..31: note = simSIRPa_10-PM
  source 1..31: mol_type = other RNA
mcmcamtmcmtmtmtmcmtmcagagmtamtg

SEQ ID NO: 191 - RNA, 21 nt, synthetic construct
  misc_feature 1..21: note = simSIRPa_10-PM
  source 1..21: mol_type = other RNA
mcamtactctgagaaagatgg

SEQ ID NO: 192 - RNA, 28 nt, synthetic construct
  misc_feature 1..28: note = simSIRPa_11-PM
  source 1..28: mol_type = other RNA
amcamtmcaamcgamcamtmcamcamta

SEQ ID NO: 193 - RNA, 20 nt, synthetic construct
  misc_feature 1..20: note = simSIRPa_11-PM
  source 1..20: mol_type = other RNA
mtatgtgatgtcgttgatgt

SEQ ID NO: 194 - RNA, 29 nt, synthetic construct
  misc_feature 1..29: note = simSIRPa_12-PM
  source 1..29: mol_type = other RNA
agaggamtamcmcmcmtmcamcmcmtamt

SEQ ID NO: 195 - RNA, 21 nt, synthetic construct
  misc_feature 1..21: note = simSIRPa_12-PM
  source 1..21: mol_type = other RNA
amtaggtgagggmtatcctct

SEQ ID NO: 196 - RNA, 28 nt, synthetic construct
  misc_feature 1..28: note = simSIRPa_13-PM
  source 1..28: mol_type = other RNA
mtmcamcamtamcgmcagamcmcmtgaa

SEQ ID NO: 197 - RNA, 21 nt, synthetic construct
  misc_feature 1..21: note = simSIRPa_13-PM
  source 1..21: mol_type = other RNA
ttmcaggtctgcgmtatgtga

SEQ ID NO: 198 - RNA, 22 nt, synthetic construct
  misc_feature 1..22: note = simSIRPa_17-PM
  source 1..22: mol_type = other RNA
gagaagaamcgmcmcagggaaa

SEQ ID NO: 199 - RNA, 20 nt, synthetic construct
  misc_feature 1..20: note = simSIRPa_17-PM
  source 1..20: mol_type = other RNA
tttcccmtggcgttcttctc

SEQ ID NO: 200 - RNA, 28 nt, synthetic construct
  misc_feature 1..28: note = simSIRPa_18-PM
  source 1..28: mol_type = other RNA
mcmcmtaamcaamcmcamcamcagaamt

SEQ ID NO: 201 - RNA, 20 nt, synthetic construct
  misc_feature 1..20: note = simSIRPa_18-PM
  source 1..20: mol_type = other RNA
attctgtgtggttgtmtagg

SEQ ID NO: 202 - RNA, 26 nt, synthetic construct
  misc_feature 1..26: note = simSIRPa_19-PM
  source 1..26: mol_type = other RNA
gmtgmtmcmcaggmtmcmcagaggaa

SEQ ID NO: 203 - RNA, 20 nt, synthetic construct
  misc_feature 1..20: note = simSIRPa_19-PM
  source 1..20: mol_type = other RNA
ttcctctggacctggamcac

SEQ ID NO: 204 - RNA, 24 nt, synthetic construct
  misc_feature 1..24: note = simSIRPa_20-PM
  source 1..24: mol_type = other RNA
aagmcamcmcgagamcaggmcaaa

SEQ ID NO: 205 - RNA, 20 nt, synthetic construct
  misc_feature 1..20: note = simSIRPa_20-PM
  source 1..20: mol_type = other RNA
tttgcctgtctmcaatgctt

SEQ ID NO: 206 - RNA, 27 nt, synthetic construct
  misc_feature 1..27: note = simSIRPa_21-PM
  source 1..27: mol_type = other RNA
mcmtaamcaamcmcamcamcagaamta

SEQ ID NO: 207 - RNA, 21 nt, synthetic construct
  misc_feature 1..21: note = simSIRPa_21-PM
  source 1..21: mol_type = other RNA
mtattctgtgtggttgtmtag

SEQ ID NO: 208 - RNA, 25 nt, synthetic construct
  misc_feature 1..25: note = simSIRPa_22-PM
  source 1..25: mol_type = other RNA
mtgmcamcgagmcmcmcgagaagaa

SEQ ID NO: 209 - RNA, 20 nt, synthetic construct
  misc_feature 1..20: note = simSIRPa_22-PM
  source 1..20: mol_type = other RNA
ttcttctcgggctcgtgmca

SEQ ID NO: 210 - RNA, 27 nt, synthetic construct
  misc_feature 1..27: note = simSIRPa_23-PM
  source 1..27: mol_type = other RNA
mcaaamtgamcamtmcaamcgamcamt

SEQ ID NO: 211 - RNA, 20 nt, synthetic construct
  misc_feature 1..20: note = simSIRPa_23-PM
  source 1..20: mol_type = other RNA
atgtcgttgatgtmcatttg

SEQ ID NO: 212 - RNA, 23 nt, synthetic construct
  misc_feature 1..23: note = simSIRPa_24-PM
  source 1..23: mol_type = other RNA
mcgagaagaamcgmcmcagggaa

SEQ ID NO: 213 - RNA, 19 nt, synthetic construct
  misc_feature 1..19: note = simSIRPa_24-PM
  source 1..19: mol_type = other RNA
ttccctggcgttcttctcg

SEQ ID NO: 214 - RNA, 26 nt, synthetic construct
  misc_feature 1..26: note = simSIRPa_25-PM
  source 1..26: mol_type = other RNA
amcaaamtgamcamtmcaamcgamca

SEQ ID NO: 215 - RNA, 20 nt, synthetic construct
  misc_feature 1..20: note = simSIRPa_25-PM
  source 1..20: mol_type = other RNA
tgtcgttgatgtmcatttgt

SEQ ID NO: 216 - RNA, 28 nt, synthetic construct
  misc_feature 1..28: note = simSIRPa_26-PM
  source 1..28: mol_type = other RNA
gamtaamtaamtgmcmtamcmcmcamca

SEQ ID NO: 217 - RNA, 23 nt, synthetic construct
  misc_feature 1..23: note = simSIRPa_26-PM
  source 1..23: mol_type = other RNA
tgtgggmtagmcatmtatmtatc

SEQ ID NO: 218 - RNA, 23 nt, synthetic construct
  misc_feature 1..23: note = simSIRPa_27-PM
  source 1..23: mol_type = other RNA
agaagaamcgmcmcagggaaamt

SEQ ID NO: 219 - RNA, 19 nt, synthetic construct
  misc_feature 1..19: note = simSIRPa_27-PM
  source 1..19: mol_type = other RNA
atttccctggcgttcttct

SEQ ID NO: 220 - RNA, 27 nt, synthetic construct
  misc_feature 1..27: note = simSIRPa_28-PM
  source 1..27: mol_type = other RNA
mtaamcaamcmcamcamcagaamtamt

SEQ ID NO: 221 - RNA, 21 nt, synthetic construct
  misc_feature 1..21: note = simSIRPa_28-PM
  source 1..21: mol_type = other RNA
amtattctgtgtggttgtmta

SEQ ID NO: 222 - RNA, 23 nt, synthetic construct
  misc_feature 1..23: note = simSIRPa_29-PM
  source 1..23: mol_type = other RNA
ggamtmcaaamcagaagaaagmc

SEQ ID NO: 223 - RNA, 19 nt, synthetic construct
  misc_feature 1..19: note = simSIRPa_29-PM
  source 1..19: mol_type = other RNA
gctttcttctgtttgatcc

SEQ ID NO: 224 - RNA, 23 nt, synthetic construct
  misc_feature 1..23: note = simSIRPa_30-PM
  source 1..23: mol_type = other RNA
tmcaaamcagaagaaagmcmcaa

SEQ ID NO: 225 - RNA, 19 nt, synthetic construct
  misc_feature 1..19: note = simSIRPa_30-PM
  source 1..19: mol_type = other RNA
ttggctttcttctgtttga

SEQ ID NO: 226 - RNA, 26 nt, synthetic construct
  misc_feature 1..26: note = simSIRPa_31-PM
  source 1..26: mol_type = other RNA
amcamcaaamtgamcamtmcaamcga

SEQ ID NO: 227 - RNA, 20 nt, synthetic construct
  misc_feature 1..20: note = simSIRPa_31-PM
  source 1..20: mol_type = other RNA
tcgttgatgtmcatttgtgt

SEQ ID NO: 228 - RNA, 28 nt, synthetic construct
  misc_feature 1..28: note = siCD45
  source 1..28: mol_type = other RNA
mcmtggmcmtgaamtmtmtmcagagmca

SEQ ID NO: 229 - RNA, 21 nt, synthetic construct
  misc_feature 1..21: note = siCD45
  source 1..21: mol_type = other RNA
tgctctgaaattmcagcmcag

(19) United States
(12) Patent Application Publication
(10) Pub. No.: US 2025/0171786 A1
(43) Pub. Date: May 29, 2025

(54) COMPOSITIONS AND METHODS FOR INHIBITING EXPRESSION OF THE SIGNAL REGULATORY PROTEIN ALPHA (SIRPa) GENE

(71) Applicants: Massachusetts Institute of Technology, Cambridge, MA (US); FUJIFILM Corporation, Tokyo (JP)

(72) Inventors: Sho TOYONAGA, Cambridge, MA (US); Theresa Marie RAIMONDO, Cambridge, MA (US); Dennis Zheng SHI, Cambridge, MA (US); Daniel Griffith ANDERSON, Cambridge, MA (US)

(73) Assignees: Massachusetts Institute of Technology, Cambridge, MA (US); FUJIFILM Corporation, Tokyo (JP)

(21) Appl. No.: 19/039,087

(22) Filed: Jan. 28, 2025

Related U.S. Application Data
(63) Continuation of application No. PCT/US2023/070941, filed on Jul. 25, 2023.
(60) Provisional application No. 63/369,926, filed on Jul. 29, 2022.

(51) Int. Cl. C12N 15/113; A61K 9/51; A61P 35/00

(52) U.S. Cl. CPC C12N 15/1138; A61K 9/5123; A61K 9/5192; A61P 35/00; C12N 2310/14; C12N 2310/321; C12N 2310/51

(57) ABSTRACT

Provided herein, in various embodiments, are methods and compositions comprising a Signal Regulatory Protein Alpha (SIRPα) therapeutic. In certain embodiments, the disclosure provides for methods and compositions for modulating SIRPα expression. In certain embodiments, the methods and compositions are useful in the treatment of a SIRPα-mediated disease or condition.

### CROSS-REFERENCE TO RELATED APPLICATIONS

This application is a continuation of PCT international application no. PCT/US2023/070941 filed on Jul. 25, 2023, which claims priority under 35 U.S.C. § 119(a) to U.S. provisional patent application No. 63/369,926 filed on Jul. 29, 2022. Each of the above application(s) is hereby expressly incorporated by reference, in its entirety, into the present application.

### INCORPORATION BY REFERENCE OF SEQUENCE LISTING

The content of the electronically submitted sequence listing contained in the following eXtensible Markup Language (XML) file being submitted concurrently herewith is incorporated herein by reference in its entirety: file name: Q299318_Sequence listing as filed.xml; created Jan. 27, 2025; 338,379 bytes in size.

### BACKGROUND

Signal Regulatory Protein Alpha (SIRPA or SIRPα), Signal Regulatory Protein Beta (SIRPB or SIRPβ), and Signal Regulatory Protein Gamma (catus or SIRPγ) are all members of the Signal Regulatory (SIRP) family of receptors and immune regulation. Members of the SIRP family have highly conserved extracellular regions but different transmembrane regions with opposite (i.e., inhibitory v. activating) signaling potentials.

Interaction of SIRPα with CD47 is an important myeloid cell immune checkpoint. The SIRPα interaction with CD47 provides a downregulatory signal that inhibits host cell phagocytosis. On the other hand, SIRPβ triggers cell activating signals through its association with the transmembrane adaptor protein DNA-X activation protein (DAP12). Thus, the SIRP family falls into a class of proteins referred to as paired receptors.

Both SIRPα and SIRPβ are expressed in myeloid lineage cells, while SIRPγ is expressed on T-cells, Natural Killer (NK) cells, and Natural Killer T (NKT) cells. SIRPβ has no known natural ligand. Both SIRPα and SIRPγ bind to CD47, although SIRPγ binds CD47 with a 10-fold weaker affinity than SIRPα. Binding of CD47 to SIRPγ has been shown to mediate adhesion between T-cells and antigen-presenting cells (APCs) and endothelial cells, resulting in T-cell activation, proliferation, and trans-endothelial migration.

Accordingly, compositions and methods for modulating the SIRPα/CD47 interaction without altering SIRPβ or SIRPγ functions are needed to treat many SIRPα-related diseases (e.g., cancer immunotherapy).

### SUMMARY

In one aspect, the disclosure provides for a method of reducing signal regulatory protein alpha (SIRPα) expression in a cell, including contacting the cell with a lipid nanoparticle composition comprising a nucleic acid that reduces expression of signal regulatory protein alpha (SIRPα). In some embodiments, the nucleic acid is a double-stranded ribonucleic acid (dsRNA), an antisense oligomer (ASO), a small interfering RNA (siRNA), a short hairpin RNA (shRNA), a micro RNA (miRNA), a circular RNA, a peptide-nucleic acid (PNA), a locked nucleic acid (LNA), or a combination thereof.

In still other aspects, the present disclosure provides for a composition including a nucleic acid that reduces expression of signal regulatory protein alpha (SIRPα), wherein the nucleic acid comprises a polynucleotide having at least 80% identity to at least one of SEQ ID NOs: 18-227.

In another aspect, the disclosure provides for a composition including: a nanocarrier selected from the group consisting of a lipid, a polymer, and a lipo-polymer hybrid, and a nucleic acid that reduces expression of signal regulatory protein alpha (SIRPα); wherein SIRPα concentration in a cell contacted with the composition is reduced compared to SIRPα concentration in an otherwise identical cell.

In some embodiments, the disclosure provides for a method of treating cancer. In some embodiments, the method is for treating a SIRPα-mediated disease or condition.

Further, the present disclosure provides for methods of making and using the methods and compositions disclosed herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows a sequence alignment between SEQ ID NO: 1 (NCBI Ref. No. NP_001035111.1), SEQ ID NO: 2 (NCBI Ref. No. NP_006056.2), and SEQ ID NO: 3 (NCBI Ref. No. NP_061026.2). SIRP family proteins share a highly conserved N-terminal extracellular domain. N-terminal signal sequences are boxed and transmembrane regions are underlined.

FIG. 2 shows a sequence alignment between SEQ ID NO: 4 (NCBI Ref. No. NM_001040022.1), SEQ ID NO: 5 (NCBI Ref. No. NM_006065.5), and SEQ ID NO: 6 (NCBI Ref. No. NM_018556.4). SIRPα siRNA target sequences are underlined and variants with minor allele frequency (MAF)>0.05 are boxed.

FIG. 3 shows macrophage-specific CD45 gene silencing in murine peritoneal immune cells.

FIG. 4 shows dose- and time-dependent SIRPα gene silencing in murine peritoneal macrophages in vivo.

FIG. 5A shows siSIRPα-LNP reduced SIRPα expression level on human primary macrophages. FIG. 5B shows quantification of FIG. 5A.

FIG. 6A shows shift in macrophage phenotype towards an ML. FIG. 6B shows shift in macrophage phenotype away from an M2 phenotype following siSIRPα-LNP silencing of SIRPα expression in primary human macrophages. FIG. 6C shows SIRPα silencing increases expression of the class I antigen presentation molecule HLA-A2 in primary human macrophages.

FIG. 7A shows siRNA sequences are capable of silencing SIRPα expression on primary human macrophages in vitro using a 1-way ANOVA with Tukey's multiple comparisons test. FIG. 7B shows that various lipids are capable of forming siSIRPα-LNPs and silencing SIRPα expression on primary human macrophages in vitro using a 2-way ANOVA with Sidak's multiple comparisons test.

FIG. 8A shows primary human macrophage viability is maintained with various siRNA sequences and lipids. FIG. 8B shows primary human macrophage viability is maintained with FL-A, MC3, and Lipid 5 lipids.

FIG. 9A shows multiple siRNAs may promote antigen presentation (HLA-DR expression). FIG. 9B shows multiple siRNAs may promote M1 polarization (CD86 expression). sihSIRPα18 is more potent than the other siRNAs. RiM=RNAiMAX, a commercial transfection reagent.

FIG. 10A shows multiple lipids are capable of forming siSIRPα-LNPs that promote the expression of antigen presentation proteins (HLA-DR). FIG. 10B shows multiple lipids are capable of forming siSIRPα-LNPs that promote the M1 phenotype protein expression (CD86).

FIG. 11A shows schematic of experimental set-up. FIG. 11B shows quantification of SIRPα silencing, as assessed by flow cytometry, within the macrophage population. Because anti-SIRPα used for blocking (as an experimental treatment) inhibited binding of the SIRPα staining antibody used in flow cytometry, SIRPα expression levels could not be determined in the anti-SIRPα condition (noted as NA in the bar graph).

FIG. 12A shows shift in macrophage phenotype towards an M1 phenotype (CD86) following siSIRPα-LNP silencing of SIRPα expression in primary human macrophages, but not following anti-SIRPα treatment. Data collected from a coculture of primary human macrophages with SKOV-3 ovarian cancer cells which had been pretreated with either anti-HER2 or IgG isotype control antibody. SKOV-3 pretreatments are shown on the x-axis, macrophage pretreatments are indicated in the legends of each bar graph. FIG. 12B shows the level of CD86 expression (media fluorescent intensity, MFI) within the CD45+ macrophage population, as measured by flow cytometry. siSIRPα-LNP+aHER2 synergistically promotes ovarian cancer phagocytosis by primary human macrophages. Blocking SIRPα by pretreating macrophages with anti-SIRPα does not improve phagocytosis, as compared to pretreatment with the isotype control antibody (noted as IgG cont.) or siSIRPα. FIG. 12C shows CFSE fluorescence intensity within the CD45+ macrophage population, quantified by flow cytometry. SIRPα silencing with siSIRPα, but not blocking with anti-SIRPα, increases expression of the class I antigen presentation molecule HLA-A2 in primary human macrophages. Data collected from a coculture of primary human macrophages with SKOV-3 ovarian cancer cells which had been pretreated with either anti-HER2 or IgG isotype control antibody.

FIG. 13 shows a schematic of experimental set-up.

FIGS. 14A-14B show blocking SIRPα with anti-SIRPα promotes mutual phagocytosis (macrophages phagocytosing other macrophages), as compared to SIRPα silencing with siSIRPα. FIG. 14A shows flow cytometric data quantifying the percentage of macrophages labeled with the violet cell tracker, as compared to their initial seeding density (40%). Violet macrophages treated with siRNA-LNP are not phagocytosed by other (green, i.e. untreated) macrophages {there is little depletion of the violet macrophage population compared to the initial seeding density}. Violet macrophages treated with anti-SIRPα or its isotype (IgG) control are phagocytosed by other macrophages (green, i.e. untreated) {substantial depletion of violet macrophages in cocultures pretreated with anti-SIRPα or the IgG control). FIG. 14B shows macrophages (violet) treated with antibodies (anti-SIRPα or its IgG control) are more actively phagocytosed by other macrophages (green) (i.e. mutual phagocytosis) than macrophages treated with siRNA-LNPs.

FIG. 15A shows SKOV-3 human ovarian cancer expresses CD47. FIG. 15B shows the quantification of FIG. 15A. FIG. 15C shows HER2 expression on SKOV-3 human ovarian cancer. FIG. 15D shows the quantification of FIG. 15C.

FIG. 16 shows a schematic representation of cancer cell phagocytosis assay.

FIG. 17A shows siSIRPα-LNP reduced SIRPα expression level on human primary macrophages. FIG. 17B shows the quantification of FIG. 17A.

FIG. 18A shows siSIRPα-LNP+aHER2 synergistically promotes ovarian cancer phagocytosis by primary human macrophages. FIG. 18B quantifies the percentage of macrophages positive for the CFSE cancer cell tracker (indicating phagocytosis).

FIGS. 19A-19C shows shift in macrophage phenotype towards an M1 and away from an M2 phenotype following siSIRPα-LNP silencing of SIRPα expression in primary human macrophages. Data collected from a coculture of primary human macrophages with SKOV-3 ovarian cancer cells which had been pretreated with either anti-HER2 or IgG isotype control antibody. FIG. 19A shows CD206. FIG. 19B shows CD163. FIG. 19C shows CD86.

FIG. 20 shows SIRPα silencing increases expression of the class II antigen presentation molecule HLA-DR in primary human macrophages. Data collected from a coculture of primary human macrophages with SKOV-3 ovarian cancer cells which had been pretreated with either anti-HER2 or IgG isotype control antibody.

FIG. 21A shows a schematic representation of macrophage cross-presentation experiment. FIG. 21B shows flow cytometry data showing SIRPα gene silencing within the macrophage population. FIG. 21C shows flow cytometry data showing M1 phenotype marker expression.

FIG. 22A shows SIRPα gene silencing increases macrophage phagocytosis of B16 melanoma cells. FIG. 22B shows cross-presentation of the model B16 melanoma antigen (OVA) on MHC class I molecules.

FIGS. 23A-23C show SIRPα gene silencing increases cross-presentation of cancer antigen by macrophages. FIG. 23A shows percent cross-presenting macrophages. FIG. 23B shows the calculated number of macrophages which were positive for both CSFE and MHC1-OVA (double positive) and FIG. 23C shows the data in FIG. 23B divided by the number of macrophages which were CSFE positive times 100.

FIGS. 24A-24B show various siRNA sequence—LNP formulation combinations are capable of silencing SIRPα expression on THP-1-derived macrophages, in vitro. FIG. 24A shows 50 nM siRNA. FIG. 24B shows 5 nM siRNA.

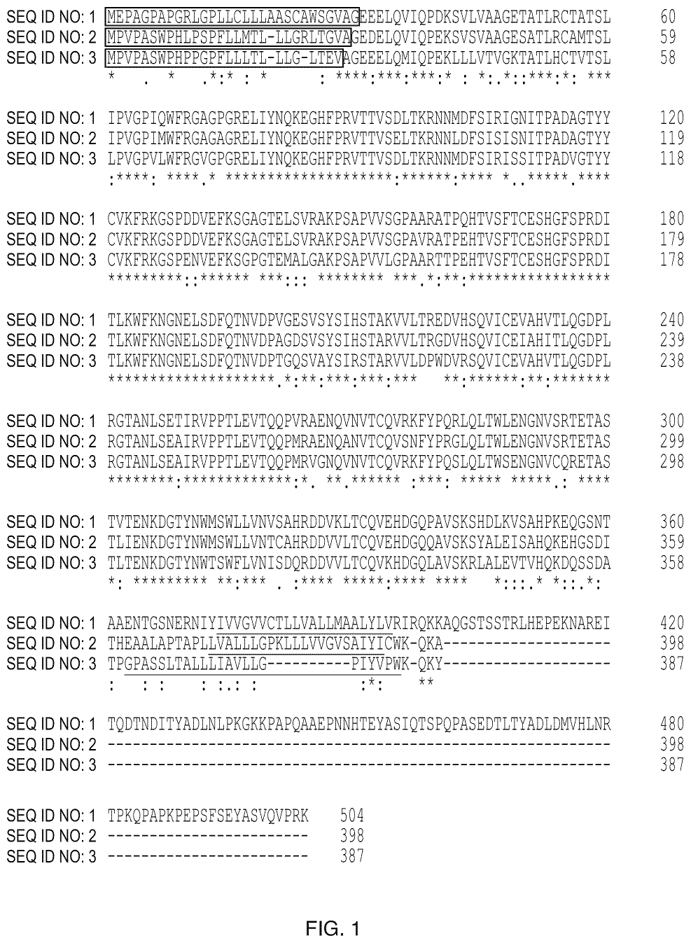

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

### Receptor Basal Activity

Some cell surface receptors have constitutive (i.e., intrinsic or basal) activity that activates intracellular signaling in the absence of a ligand. This basal activity is defined as the probability of receptors that exist in the active state in the absence of ligand. Although antibody therapeutics have been shown to be useful to sterically block receptor-ligand interactions, antibody therapeutics are not thought to be able to suppress receptor basal activity.

Nucleic acid therapeutics, such as siRNA therapeutics, represent a promising approach because such therapies can reduce the number of receptors on the cell surface as well as the constitutive signaling.

### Signal Regulatory Protein Alpha (SIRPα)

Signal regulatory protein alpha or SIRPα (also designated as CD172a or SHPS-1) is a transmembrane protein expressed on myeloid cells.

The SIRPα-CD47 immune checkpoint has been the target of numerous blocking agents (e.g., anti-CD47 and anti-SIRPα antibodies and SIRPα fusion proteins), several of which are being evaluated in clinical trials (e.g., Hu5F9-G4, TTI-621 and ALX148).

However, while these therapies sterically block the CD47-SIRPα and work well in combination with antibodies that opsonize a target cell, neither the blockade of CD47-SIRPα nor the opsonization of a target is sufficient to make target cell macrophage engulfment efficient. Moreover, use of multiple blocking agents has been shown to cause heterotrimeric interactions that have been described as the “scorpion effect” (Kurlander, R. J. “Blockade of Fc receptor-mediated binding to U-937 cells by murine monoclonal antibodies directed against a variety of surface antigens.” The Journal of Immunology 131.1 (1983): 140-147) (herein incorporated by reference in its entirety). In addition, anti-SIRPα antibodies cause opsonization of macrophages, resulting in mutual phagocytosis, where macrophages are phagocytosed by other macrophages.

Moreover, it has been reported that the gene encoding human SIRPα is highly polymorphic. This is also true for the extracellular domains of SIRPα. Thus, effective therapeutic targeting of SIRPα across diverse patient populations requires pan-allelic SIRPα inhibition while maintaining SIRPα specificity over the other SIRP family molecules.

Thus, there exists a need to develop therapeutics that can be designed to cover polymorphic variability while avoiding regions with high allele frequency.

One such approach is the use of nucleic acid therapeutics such as siRNA against SIRPα. siRNAs against SIRPα has been shown to promote macrophage polarization from anti-inflammatory M2 to pro-inflammatory M1 phenotype; however, presently available siRNAs against SIRPα have not been optimized for specificity and potency, and, to date no siRNA against SIRPα has been formulated in lipid nanoparticles for in vivo or clinical use.

Therefore, there is a strong demand for nucleic acid therapeutic compositions and methods designed to target transmembrane, cytoplasmic region, or even untranslated regions of mRNA. Moreover, by combining nucleic acid therapeutics with appropriate delivery vehicles, such as lipid nanoparticles, cell-type specific delivery of nucleic acid therapeutics may be achieved, reducing off-target effects.

### Compositions of the Disclosure

In some embodiments, the disclosure provides for a composition that is a pharmaceutically acceptable composition.

In one aspect, the present disclosure provides for a composition comprising a nucleic acid that reduces expression of signal regulatory protein alpha (SIRPα), wherein the nucleic acid comprises a polynucleotide having at least 80% identity to at least one of SEQ ID NOs: 18-227. In some embodiments, the polynucleotide is at least 80% identical to at least one of SEQ ID NOs: 140-227.

As used herein, the term “sequence identity,” refers to the extent to which two sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology). In some embodiments, codon-optimized sequences for efficient expression in different cells, tissues, and/or organisms reflect the pattern of codon usage in such cells, tissues, and/or organisms containing conservative (or non-conservative) amino acid substitutions that do not adversely affect normal activity.

Accordingly, in some embodiments, the polynucleotide is at least about 70% identical to at least one of SEQ ID NOs: 18-227, for example, has at least about: 75%, 80%, 85%, 90%, 95%, 96%, 97%, 98%, 99% or 100% sequence identity to at least one of SEQ ID NOs: 18-227. In certain embodiments, the polynucleotide is about: 70%, 75%, 80%, 85%, 90%, 95%, 96%, 97%, 98%, 99% or 100% identical to at least one of SEQ ID NOs: 18-227. In some embodiments, the polynucleotide about 70%, 75%, 80%, 85%, 90%, 95%, 96%, 97%, 98%, 99% or 100% sequence identity to at least one of SEQ ID NOs: 18-227. In particular embodiments, the polynucleotide is about 70-100% sequence identity to at least one of SEQ ID NOs: 18-227, for example, about: 75-100%, 75-99%, 80-100%, 80-98%, 85-100%, 85-97%, 90-100%, 90-96%, 95-100%, 96-100%, 97-100%, 98-100% or 99-100%.

In still further embodiments, the polynucleotide is at least about 70% identical to at least one of SEQ ID NOs: 140-227, for example, has at least about: 75%, 80%, 85%, 90%, 95%, 96%, 97%, 98%, 99% or 100% sequence identity to at least one of SEQ ID NOs: 140-227. In certain embodiments, the polynucleotide is about: 70%, 75%, 80%, 85%, 90%, 95%, 96%, 97%, 98%, 99% or 100% identical to at least one of SEQ ID NOs: 140-227. In some embodiments, the polynucleotide about 70%, 75%, 80%, 85%, 90%, 95%, 96%, 97%, 98%, 99% or 100% sequence identity to at least one of SEQ ID NOs: 140-227. In particular embodiments, the polynucleotide is about 70-100% sequence identity to at least one of SEQ ID NOs: 140-227, for example, about: 75-100%, 75-99%, 80-100%, 80-98%, 85-100%, 85-97%, 90-100%, 90-96%, 95-100%, 96-100%, 97-100%, 98-100% or 99-100%.

In some embodiments, the composition of the disclosure, wherein contacting a cell with the nucleic acid reduces the concentration of SIRPα compared to the SIRPα concentration in an otherwise identical cell.

As used herein, the term “reducing” or “reduce” refers to modulation that decreases the concentration of SIRPα (e.g., the level prior to or in an absence of modulation by the agent). In some embodiments, the agent (e.g., composition) reduces the concentration of SIRPα, by at least about 5%, e.g., by at least about: 10%, 15%, 20%, 25%, 30%, 35%, 40%, 45%, 50%, 55%, 60%, 65%, 70%, 75%, 80%, 85%, 90%, 95% or 98%. In certain embodiments, the agent (e.g., composition) decreases the concentration of SIRPα, by at least about 5%, e.g., by at least about: 10%, 15%, 20%, 25%, 30%, 35%, 40%, 45%, 50%, 55%, 60%, 65%, 70%, 75%, 80%, 85%, 90%, 95% or 98%. In particular embodiments, the agent (e.g., composition) decreases the concentration of SIRPα, by at least about 5%, e.g., by at least about: 10%, 15%, 20%, 25%, 30%, 35%, 40%, 45%, 50%, 55%, 60%, 65%, 70%, 75%, 80%, 85%, 90%, 95% or 98%.

In some embodiments, the concentration of SIRPα is measured using a cell-based functional assay.

The present disclosure also provides for a composition comprising: a nanocarrier selected from the group consisting of a lipid, a polymer, and a lipo-polymer hybrid, and a nucleic acid signal regulatory protein alpha (SIRPα) therapeutic; wherein SIRPα concentration in a cell contacted with the composition is reduced compared to SIRPα concentration in an otherwise identical cell. In some embodiments, the SIRPα therapeutic is a double-stranded ribonucleic acid (dsRNA), an antisense oligomer (ASO), a small interfering RNA (siRNA), a short hairpin RNA (shRNA), a micro RNA (miRNA), a circular RNA, a peptide-nucleic acid (PNA), a locked nucleic acid (LNA), or a combination thereof.

In certain embodiments of the disclosure, the composition wherein the SIRPα therapeutic comprises a polynucleotide having at least about 80% identity to a contiguous sequence within SEQ ID NO:4, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17, or a combination thereof, wherein the contiguous nucleotide sequence is at least about 15 nucleotides in length. In still further embodiments, the composition wherein the SIRPα therapeutic comprises a polynucleotide having at least about 80% identity to a contiguous sequence within SEQ ID NO:4, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17, or a combination thereof, wherein the contiguous nucleotide sequence is about 15-30 nucleotides in length. In still further embodiments, the composition wherein the SIRPα therapeutic comprises a polynucleotide having at least about 80% identity to at least one of SEQ ID NOs: 18-139.

In some embodiments, the composition further comprises a 2′-deoxythymidine-3′-phosphate 3′ overhang or a 2′-deoxythymidine-5′-phosphate-phosphorothioate 3′ overhang. In still further embodiments, the polynucleotide sequence further comprises at least one modified pyrimidine. The modified pyrimidine may be 2′-O-methylcytidine-3′-phosphate or 2′-O-methyluridine-3′-phosphate. In some embodiments, the SIRPα therapeutic is an siRNA duplex. In some embodiments, the siRNA duplex comprises the polynucleotide sequence hybridized to a complementary sequence.

In some embodiments, the concentration of the SIRPα therapeutic is at least about 25 nM, 2.5 nM, 250 pM, or 25 pM. In some embodiments, the composition is formulated as a pharmaceutical composition. In still further embodiments, the pharmaceutical composition is an advanced therapy medicinal product. Examples of advanced therapy medicinal products include but are not limited to somatic cell therapy medicinal products, tissue-engineered products, gene therapy medicinal products, tumor vaccines, or combinations thereof.

### Methods of the Disclosure

In some embodiments, the disclosure provides for methods of treatment and methods of enhancing efficacy of treatment comprising administration of the compositions described herein.

As used herein, “therapy,” “treat,” “treating,” or “treatment” means inhibiting or relieving a condition in a subject in need thereof. For example, a therapy or treatment refers to any of: (i) the prevention of symptoms associated with a disease or disorder; (ii) the postponement of development of the symptoms associated with a disease or disorder; and/or (iii) the reduction in the severity of such symptoms that will, or are expected, to develop with said disease or disorder. The terms include ameliorating or managing existing symptoms, preventing additional symptoms, and ameliorating or preventing the underlying causes of such symptoms. Thus, the terms denote that a beneficial result is being conferred on at least some of the subjects (e.g., humans) being treated. Many therapies or treatments are effective for some, but not all, subjects that undergo the therapy or treatment.

As used herein, the term “effective amount” means an amount of a composition, that when administered alone or in combination to a cell, tissue, or subject, is effective to achieve the desired therapy or treatment under the conditions of administration. For example, an effective amount is one that would be sufficient to produce an immune response to bring about effectiveness of a therapy or treatment. The effectiveness of a therapy or treatment (e.g., eliciting a humoral and/or cellular immune response) can be determined by suitable methods known in the art.

In some embodiments, the disclosure provides for the use of a composition or agent for treating a signal regulatory protein alpha (SIRPα)-mediated disease or condition. In some embodiments, the disclosure provides for a method of reducing signal regulatory protein alpha (SIRPα) expression in a cell, comprising contacting the cell with a lipid nanoparticle composition comprising a nucleic acid signal regulatory protein alpha (SIRPα) therapeutic.

As used herein, “subject” or “patient” includes humans, domestic animals, such as laboratory animals (e.g., dogs, monkeys, pigs, rats, mice, etc.), household pets (e.g., cats, dogs, rabbits, etc.) and livestock (e.g., chickens, pigs, cattle (e.g., a cow, bull, steer, or heifer), sheep, goats, horses, etc.), and non-domestic animals. In some embodiments, a subject is a mammal (e.g., a non-human mammal). In some embodiments, a subject is a human. In still further embodiments, a subject of the disclosure may be a cell, cell culture, tissue, organ, or organ system. In some embodiments, the subject is male. In some embodiments, the subject is female. In some embodiments, the subject is an infant, a juvenile, an adolescent, or an adult.

As used herein, “administering” or “administration” refers to taking steps to deliver a composition to a subject. Administering can be performed, for example, once, a plurality of times, and/or over one or more extended periods. Administration includes both direct administration, including self-administration, and indirect administration, including the act of prescribing or directing a subject to consume a composition. For example, as used herein, one (e.g., a physician) who instructs a subject (e.g., a patient) to self-administer a composition (e.g., a drug), or to have the composition administered by another and/or who provides a subject with a prescription for a composition is administering the composition to the subject.

In some aspects, the disclosure provides for a method of treating a signal regulatory protein alpha (SIRPα)-mediated disease or condition, comprising administering to subject in need thereof a lipid nanoparticle (LNP) composition comprising a nucleic acid signal regulatory protein alpha (SIRPα) therapeutic.

In still further aspects, the disclosure provides for a method of treating cancer, comprising administering to subject in need thereof a lipid nanoparticle (LNP) composition comprising a nucleic acid signal regulatory protein alpha (SIRPα) therapeutic. In some embodiments of the disclosure, the methods relate to treatment of cancer. Cancer treatment provided by the disclosure includes carcinoma, sarcoma, melanoma, lymphoma, and/or leukemia. In some embodiments, the method of the disclosure may replace, proceed, or follow another treatment regimen. In some embodiments, another treatment regimen may include, but is not limited to, chemotherapy, radiation therapy, surgery, hormone therapy, biological response modifier therapy, immunotherapy, and/or bone marrow transplant.

In some aspects, the methods of the disclosure provide for treatment of a SIRPα-mediated disease or condition. Examples of SIRPα-mediated diseases and/or conditions include but are not limited to acute myeloid leukemia, adenosquamous lung carcinoma, atypical meningioma, B-cell acute lymphoblastic leukemia, basal cell carcinoma, bile duct carcinoma, bladder carcinoma, bladder transitional cell carcinoma, brain glioblastoma, breast carcinoma, breast ductal adenocarcinoma, Burkitts lymphoma, cecum adenocarcinoma, cervical squamous cell carcinoma, chronic lymphosytic leukemia, clear cell renal carcinoma, colon adenocarcinoma, cutaneous melanoma, endometrial endometrioid adenocarcinoma, esophageal adenocarcinoma, esophageal squamous cell carcinoma, gastric adenocarcinoma, gastric carcinoma, glioma, head and neck squamous cell carcinoma, hepatobiliary neoplasm, hepatoblastoma, hepatocellular carcina, HER2 positive breast carcinoma, kidney neoplasm, large cell lung carcinoma, lobular breast carcinoma, lunch adenocarcinoma, lung carcinoma, lymphoid neoplasm, melanoma, multiple myeloma, nasopharyngeal squamous cell carcinoma, non-small cell lung carcinoma, oral squamous cell carcinoma, ovarian endometroid adenocarcinoma with squamous differentiation, ovarian serous adenocarcinoma, pancreatic acinar cell carcinoma, pancreatic carcinoma, pancreatic ductal adenocarcinoma, pancreatic neuroendocrine tumor, papillary renal cell carcinoma, prostate adenocarcinoma, rectal adenocarcinoma, skin carcinoma, small cell lung carcinoma, squamous cell lung carcinoma, thyroid carcinoma, thyroid neoplasm, and uterine carcinosarcoma.

In some embodiments, the disclosure provides for a composition that is a pharmaceutically acceptable composition.

As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion). In some embodiments, the intervals may be daily, weekly, biweekly, monthly, quarterly, every two or three months, once a year, or twice a year, or any combination thereof.

Determining the dosage and route of administration for a particular agent, patient and disease or condition is well within the abilities of one of skill in the art. Preferably, the dosage does not cause or produces minimal adverse side effects.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject's disposition to the disease, condition or symptoms, the judgment of the treating physician and the severity of the particular disease being treated. The amount of an agent in a composition will also depend upon the particular agent in the composition.

In some embodiments, the concentration of one or more active agents provided in a composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v; and/or greater than 90%, 80%, 70%, 60%, 50%, 40%, 30%, 20%, 10%, 5%, 1%, 0.5%, 0.4%, 0.3%, 0.2%, 0.1%, or 0.01% w/w, w/v, or v/v.

In some embodiments, the concentration of one or more active agents provided in a composition is in the range from about 0.01% to about 50%, about 0.01% to about 40%, about 0.01% to about 30%, about 0.05% to about 25%, about 0.1% to about 20%, about 0.15% to about 15%, or about 1% to about 10% w/w, w/v or v/v. In some embodiments, the concentration of one or more active agents provided in a composition is in the range from about 0.001% to about 10%, about 0.01% to about 5%, about 0.05% to about 2.5%, or about 0.1% to about 1% w/w, w/v or v/v.

In certain embodiments, the administration of the composition may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intramuscular or subcutaneous injection. The compositions may be injected, for instance, directly into a tumor, lymph node, tissue, organ, or site of infection.

In some embodiments, compositions as described herein are used in combination with other known agents and therapies. Administered “in combination”, as used herein, means that two (or more) different treatments are delivered to the subject during the course of the subject's treatment e.g., the two or more treatments are delivered after the subject has been diagnosed with the disease and before the disease has been cured or eliminated or treatment has ceased for other reasons. In some embodiments, different treatments (e.g., additional therapeutics) can be administered simultaneously or sequentially.

In some embodiments, the disclosure provides for a method of making a lipid nanoparticle formulation comprising: obtaining a signal regulatory protein alpha (SIRPα) therapeutic, wherein the SIRPα therapeutic comprises a polynucleotide having at least about 80% identity to a contiguous sequence SEQ ID NO:4, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17; diluting the polynucleotide in citrate buffer to form an aqueous phase; solubilizing a mixture of ionizable lipid, 1,2-distearoyl-sn-glycero-3-phosphocholine (DSPC), cholesterol and 1,2-dimyristoyl-rac-glycero-3-methoxypolyethylene glycol-2000 (DMG-PEG2000) to form an ethanol phase; mixing the aqueous phase with the ethanol phase, wherein a precipitate is formed; separating the precipitate to obtain a lipid nanoparticle formulation.

In some embodiments, the disclosure provides for compositions and methods for altering the homeostatic control mechanism to modulate phagocytic cell activity.

In some embodiments, the disclosure provides for compositions and methods for macrophage activation. In some embodiments, macrophage activation: increases the inflammatory phenotype of the myeloid cells by: i) increased expression and/or secretion of cluster of differentiation 80 (CD80), CD86, MHCII, MHCI, interleukin 1-beta (IL-1b), IL-6, CCL3, CCL4, CXCL10, CXCL9, GM-CSF and/or tumor necrosis factor alpha (TNF-α); ii) decreased expression and/or secretion of CD206, CD163, CD16, CD53, VSIG4, PSGL-1, TGFb and/or IL-10; iii) increased secretion of at least one cytokine or chemokine selected from the group consisting of IL-1b, TNF-α, IL-12, IL-18, GM-CSF, CCL3, CCL4, and IL-23; iv) increased ratio of expression of IL-1b, IL-6, and/or TNF-α to expression of IL-10; v) increased CD8+ cytotoxic T cell activation; vi) increased recruitment of CD8+ cytotoxic T cell activation; vii) increased CD4+ helper T cell activity; viii) increased recruitment of CD4+ helper T cell activity; ix) increased NK cell activity; x) increased recruitment of NK cell; xi) increased neutrophil activity; xii) increased macrophage and/or dendritic cell activity; and/or xiii) increased spindle-shaped morphology, flatness of appearance, and/or number of dendrites, as assessed by microscopy.

### Lipid Nanoparticle (LNP)

In some embodiments of the disclosure, the composition is a lipid nanoparticle composition. The lipid nanoparticle composition comprises an ionizable lipid, phospholipid, sterol, polymer conjugated lipid, or any combination thereof. In embodiments of the disclosure, the amount of the ionizable lipid with respect to the total mass of the composition is about 20 mol % to about 80 mol %, about 35 mol % to about 70 mol %, or about 40 mol % to about 65 mol %. The amount of the cholesterol in with respect to the total mass of the composition is about 10 mol % to about 60 mol %, about 20 mol % to about 55 mol %, or about 25 mol % to about 50 mol %.

In some embodiments of the disclosure, the amount of the phospholipid with respect to the total mass of the composition is about 3 mol % to about 55 mol %. The amount of the phospholipid with respect to the total mass of the composition is, e.g., about 0.25 mol % to about 12 mol %, about 0.5 mol % to about 6 mol %, or about 1 mol % to about 3 mol %.

Sterols are disclosed in US 2023/0210993 A1. Examples of sterols include cholesterol, phytosterol (sitosterol, stigmasterol, fucosterol, spinasterol, brassicasterol, and the like), ergosterol, cholestanone, cholestenone, coprostanol, cholesteryl-2′-hydroxyethyl ether, and cholesteryl-4′-hydroxybutyl ether.

Polymer conjugated lipid are disclosed in WO 2023/114943 A2. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

### Ionizable Lipids

In some embodiments of the disclosure, the ionizable lipid is a lipid having at least one biodegradable group. The ionizable lipid may be a lipid having at least one ionizable amino group and at least one biodegradable group. Examples of the above-mentioned biodegradable group include groups represented by —O (CO)O—, —O (CO)—, or —(CO) O—.

In some embodiments, lipid compositions include those of US 2022/0096381, the contents of which are herein incorporated by reference. For example, a lipid represented by Formula (1) or a salt thereof may be used as the ionizable lipid:

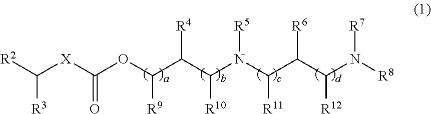

In the formula, X represents —NR¹— or —O—, R¹ represents a hydrogen atom, a hydrocarbon group having 6 to 24 carbon atoms, or a group represented by R²¹-L¹-R²²—, where R²¹ represents a hydrocarbon group having 1 to 24 carbon atoms, L¹ represents —O(CO)O—, —O(CO)—, —(CO)O—, —O—, or a group represented by the following formula,

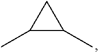

and R²² represents a divalent hydrocarbon linking group having 1 to 18 carbon atoms, R² and R³ each independently represent a hydrogen atom, a hydrocarbon group having 3 to 24 carbon atoms, or a group represented by R³¹-L²-R³²—, where R³¹ represents a hydrocarbon group having 1 to 24 carbon atoms, L² represents —O(CO)O—, —O(CO)—, —(CO)O—, —O—, or a group represented by the following formula,

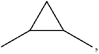

and R³² represents a divalent hydrocarbon linking group having 1 to 18 carbon atoms, R⁴, R⁵, R⁶, R⁷, R⁸, R⁹, R¹⁰, R¹¹, and R¹² each independently represent a hydrogen atom or an alkyl group having 1 to 18 carbon atoms which may be substituted, groups in any one or more pairs among R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring which may contain an O atom, a substituent on the alkyl group having 1 to 18 carbon atoms which may be substituted is a hydroxyl group, a carboxyl group, an amino group represented by —NR⁴⁵R⁴⁶, a substituted or unsubstituted aryl group, a substituted or unsubstituted heteroaryl group, or a group represented
by —O(CO)O—R⁴¹, —O(CO)—R⁴², —(CO)O—R⁴³, or —O—R⁴, where R⁴¹, R⁴², R⁴³, R⁴⁴, R⁴⁵, and R⁴⁶ each independently represent a hydrocarbon group having 1 to 18 carbon atoms, the substituent on the substituted or unsubstituted aryl group and on the substituted or unsubstituted heteroaryl group is an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴, where R⁴¹, R⁴², R⁴³, R⁴⁴, R⁴⁵, and R⁴⁶ each independently represent a hydrocarbon group having 1 to 18 carbon atoms, and a, b, c, and d each independently represent an integer of 0 to 3, a+b is 1 or more, and c+d is 1 or more.

In some embodiments, R1 represents a hydrocarbon group having 6 to 24 carbon atoms. R2 and R3 represent a hydrocarbon group having 3 to 24 carbon atoms, wherein the hydrocarbon group is an alkyl group, an alkenyl group, an alkynyl group, an alkyl group, or an alkenyl group. As the hydrocarbon group having 6 to 24 carbon atoms that is represented by R¹ and the hydrocarbon group having 3 to 24 carbon atoms that is represented by R² and R³, an alkyl group, an alkenyl group, an alkynyl group, an alkyl group, or an alkenyl group. The alkyl group having 6 to 24 carbon atoms and the alkyl group having 3 to 24 carbon atoms may be linear or branched or may be chainlike or cyclic.

The alkyl group having 6 to 24 carbon atoms may be an alkyl group having 6 to 20 carbon atoms, and the alkyl group having 3 to 24 carbon atoms may be an alkyl group having 6 to 20 carbon atoms. Specifically, examples thereof include a hexyl group, a heptyl group, an octyl group, a nonyl group, a decyl group, an undecyl group, a dodecyl group, a tridecyl group, a trimethyldodecyl group (e.g., a 3,7,11-trimethyldodecyl group), a tetradecyl group, a pentadecyl group, a hexadecyl group, a tetramethylhexadecyl group (e.g., a 3,7,11,15-tetramethylhexadecyl group), a heptadecyl group, an octadecyl group, a nonadecyl group, an icosyl group, and the like.

The alkenyl group having 6 to 24 carbon atoms and the alkenyl group having 3 to 24 carbon atoms may be linear or branched or may be chainlike or cyclic. The alkenyl group having 6 to 24 carbon atoms may be an alkenyl group having 6 to 20 carbon atoms, and the alkenyl group having 3 to 24 carbon atoms may be an alkenyl group having 6 to 20 carbon atoms. Specifically, examples thereof include a hexenyl group, a heptenyl group, an octenyl group, a nonenyl group, a decenyl group, an undecenyl group, a dodecenyl group, a dodecadienyl group, a tridecenyl group, a tetradecenyl group, a pentadecenyl group, a hexadecenyl group (e.g., a (Z)-hexadec-9-enyl group), a hexadecadienyl group, a heptadecenyl group (e.g., a (Z)-heptadec-8-enyl group), a heptadecadienyl group (e.g., a (8Z,11Z)-heptadeca-8,11-dienyl group), an octadecenyl group (e.g., a (Z)-octadec-9-enyl group), an octadecadienyl group (e.g., a (9Z,12Z)-octadeca-9,12-dienyl group), a nonadecenyl group, an icosenyl group (e.g., a (Z)-icos-11-enyl group), an icosadienyl group (e.g., a (11Z,14Z)-icosa-11,14-dienyl group), and the like.

The alkynyl group having 6 to 24 carbon atoms may be an alkynyl group having 6 to 20 carbon atoms, and the alkynyl group having 3 to 24 carbon atoms may be an alkynyl group having 6 to 20 carbon atoms. Specifically, examples thereof include a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like. All of the above alkenyl groups may have one double bond or two double bonds. All of the above alkynyl groups may have one triple bond or two triple bonds.

The hydrocarbon group having 1 to 24 carbon atoms that is represented by R²¹ and R³ may be an alkyl group having 10 to 24 carbon atoms, an alkenyl group having 10 to 24 carbon atoms, or an alkynyl group having 10 to 24 carbon atoms. The alkyl group having 10 to 24 carbon atoms may be linear or branched or may be chainlike or cyclic. The alkyl group having 10 to 24 carbon atoms may be an alkyl group having 12 to 24 carbon atoms. Specifically, examples thereof include a decyl group, an undecyl group, a dodecyl group, a tridecyl group, a trimethyldodecyl group (e.g., a 3,7,11-trimethyldodecyl group), a tetradecyl group, a pentadecyl group, a hexadecyl group, a tetramethylhexadecyl group (e.g., a 3,7,11,15-tetramethylhexadecyl group), a heptadecyl group, an octadecyl group, a 2-butylhexyl group, a 2-butyloctyl group, a 1-pentylhexyl group, a 2-pentylheptyl group, a 3-pentyloctyl group, a 1-hexylheptyl group, a 1-hexylnonyl group, a 2-hexyloctyl group, a 2-hexyldecyl group, a 3-hexylnonyl group, a 1-heptyloctyl group, a 2-heptylnonyl group, a 2-heptylundecyl group, a 3-heptyldecyl group, a 1-octylnonyl group, a 2-octyldecyl group, a 2-octyldodecyl group, a 3-octylundecyl group, a 2-nonylundecyl group, a 3-nonyldodecyl group, a 2-decyldodecyl group, a 2-decyltetradecyl group, a 3-decyltridecyl group, a 2-(4,4-dimethylpentan-2-yl)-5,7,7-trimethyloctyl group, and the like. The alkenyl group having 10 to 24 carbon atoms may be linear or branched or may be chainlike or cyclic. Specifically, examples thereof include a decenyl group, an undecenyl group, a dodecenyl group, a dodecadienyl group, tridecenyl group (e.g., a (Z)-tridec-8-enyl group), a tetradecenyl group (e.g., a tetradec-9-enyl group), a pentadecenyl group (e.g., a (Z)-pentadec-8-enyl group), a hexadecenyl group (e.g., a (Z)-hexadec-9-enyl group), a hexadecadienyl group, a heptadecenyl group (e.g., a (Z)-heptadec-8-enyl group), a heptadecadienyl group (e.g., a (8Z,11Z)-heptadeca-8,11-dienyl group), an octadecenyl group (e.g., a (Z)-octadec-9-enyl group), an octadecadienyl group (e.g., a (9Z,12Z)-octadeca-9,12-dienyl group), and the like. The alkynyl group having 10 to 24 carbon atoms may be linear or branched or may be chainlike or cyclic. Specifically, examples thereof include a decynyl group, an undecynyl group, a dodecynyl group, a tetradecynyl group, a pentadecynyl group, a hexadecynyl group, a heptadecynyl group, an octadecynyl group, and the like. All of the above alkenyl groups may have one double bond or two double bonds. All of the above alkynyl groups may have one triple bond or two triple bonds.

The divalent hydrocarbon linking group having 1 to 18 carbon atoms that is represented by R²² and R³² may be an alkylene group having 1 to 18 carbon atoms or an alkenylene group having 2 to 18 carbon atoms. The alkylene group having 1 to 18 carbon atoms may be linear or branched or may be chainlike or cyclic. The number of carbon atoms in the alkylene group may be 1 to 12, 1 to 10, or 2 to 10. Specifically, examples thereof include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, a dodecamethylene group, and the like. The alkenylene group having 2 to 18 carbon atoms may be linear or branched or may be chainlike or cyclic. The number of carbon atoms in the alkenylene group may be 1 to 12, or 2 to 10.

—O(CO)O—, —O(CO)—, and —(CO)O— may be in a range of L¹, and —O(CO)— and —(CO)O— may be in a range of L¹. —O(CO)O—, —O(CO)—, and —(CO)O— may be in a range of L², and —O(CO)— and —(CO)O— may be in a range of L². The alkyl group having 1 to 18 carbon atoms which may be substituted and which represented by R⁴, R⁶, R⁹, R¹⁰, R¹¹, and R¹² may be linear or branched or may be chainlike or cyclic. The number of carbon atoms in the alkyl group may be 1 to 12. Specifically, examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, as the substituent, a hydroxyl group, a carboxyl group, or a group represented by —O(CO)O—R⁴¹, —O(CO)—R⁴², —(CO)O—R⁴³, or —O—R⁴⁴, a group represented by —O(CO)—R⁴² or —(CO)O—R⁴³.

The alkyl group having 1 to 18 carbon atoms which may be substituted and which represented by R⁵, R⁷, and R⁸ may be linear or branched or may be chainlike or cyclic. The number of carbon atoms in the alkyl group may be 1 to 12, or may be 1 to 8. Specifically, examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, as the substituent, a hydroxyl group, a carboxyl group, or a group represented by —O(CO)O—R⁴¹, —O(CO)—R⁴², —(CO)O—R⁴³, or —O—R⁴⁴, or a group represented by —O(CO)—R⁴², —(CO)O—R⁴³, or —O—R⁴.

Examples of the 4- to 7-membered ring which may contain an O atom include an azetidine ring, a pyrrolidine ring, a piperidine ring, a morpholine ring, and an azepane ring. The 4- to 7-membered ring may be a 6-membered ring, a piperidine ring, or a morpholine ring.

In a case where the alkyl group having 1 to 18 carbon atoms which is represented by R⁴, R⁵, R⁶, R⁷, R⁸, R⁹, R¹⁰, R¹¹, and R¹² and which may be substituted has a substituted or unsubstituted aryl group as a substituent, the number of carbon atoms in the aryl group may be 6 to 22, 6 to 18, or 6 to 10. Specifically, examples of the aryl group include a phenyl group, a naphthyl group, an anthracenyl group, a phenanthrenyl group, and the like. As the substituent on the aryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴, a hydroxyl group, or a carboxyl group. Specifically, examples of the substituted aryl group include a hydroxyphenyl group, a carboxyphenyl group, and the like.

In a case where the alkyl group having 1 to 18 carbon atoms which is represented by R⁴, R⁵, R⁶, R⁷, R⁸, R⁹, R¹⁰, R¹¹, and R¹² and which may be substituted has a substituted or unsubstituted heteroaryl group as a substituent, the number of carbon atoms in the heteroaryl group is 1 to 12, or 1 to 6. Specifically, examples of the heteroaryl group include a pyridyl group, a pyrazolyl group, an imidazolyl group, a benzimidazolyl group, a thiazolyl group, an oxazolyl group, and the like. As the substituent on the heteroaryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴, a hydroxyl group, or a carboxyl group. Specifically, examples of the substituted or unsubstituted heteroaryl group include a hydroxypyridyl group, a carboxypyridyl group, a pyridonyl group, and the like.

As hydrocarbon group having 1 to 18 carbon atoms that is represented by R⁴¹, R⁴², R⁴³, R⁴⁴, R⁴⁵, and R⁴⁶, an alkyl group having 1 to 18 carbon atoms, an alkenyl group having 2 to 18 carbon atoms, or an alkynyl group having 2 to 18 carbon atoms, and an alkyl group having 1 to 18 carbon atoms, or an alkenyl group having 2 to 18 carbon atoms. The alkyl group having 1 to 18 carbon atoms may be linear or branched or may be chainlike or cyclic. The number of carbon atoms in the alkyl group is 3 to 18, or 5 to 18. Specifically, examples thereof include a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, a tridecyl group, a trimethyldodecyl group (e.g., a 3,7,11-trimethyldodecyl group), a tetradecyl group, a pentadecyl group, a hexadecyl group, a heptadecyl group, an octadecyl group, and the like. The alkenyl group having 2 to 18 carbon atoms may be linear or branched or may be chainlike or cyclic. The number of carbon atoms in the alkenyl group is 3 to 18, or 5 to 18. Specifically, examples thereof include an allyl group, a prenyl group, a pentenyl group, a hexenyl group, a heptenyl group, an octenyl group, a nonenyl group (e.g., a (Z)-2-nonenyl group or an (E)-2-nonenyl group), a decenyl group, an undecenyl group, a dodecenyl group, a dodecadienyl group, a tridecenyl group (e.g., a (Z)-tridec-8-enyl group), a tetradecenyl group (e.g., a tetradec-9-enyl group), a pentadecenyl group (e.g., a (Z)-pentadec-8-enyl group), a hexadecenyl group (e.g., a (Z)-hexadec-9-enyl group), a hexadecadienyl group, a heptadecenyl group (e.g., a (Z)-heptadec-8-enyl group), a heptadecadienyl group (e.g., a (8Z,11Z)-heptadeca-8,11-dienyl group), an octadecenyl group (e.g., a (Z)-octadec-9-enyl group), an octadecadienyl group (e.g., a (9Z,12Z)-octadeca-9,12-dienyl group), and the like. The alkynyl group having 2 to 18 carbon atoms may be linear or branched or may be chainlike or cyclic. The number of carbon atoms in the alkynyl group is 3 to 18, or 5 to 18. Specifically, examples thereof include a propargyl group, a butynyl group, a pentynyl group, a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like.

In a case where X represents —NR¹—, R¹ may represent a hydrocarbon group having 6 to 24 carbon atoms or a group represented by R²¹-L¹-R²²—. In this case, it may be that one of R² and R³ represent a hydrogen atom and the other represent a hydrocarbon group having 6 to 24 carbon atoms or a group represented by R³¹-L²-R³²—.

In a case where X represents —O—, it may be that R² and R³ each independently represent a hydrocarbon group having 6 to 24 carbon atoms or a group represented by R³¹-L²-R³²—. It may be that R⁴, R⁶, R⁹, R¹⁰, R¹¹, and R¹² each represent a hydrogen atom. R⁵ may be a hydrogen atom, an alkyl group having 1 to 18 carbon atoms, an alkyl group having 1 to 18 carbon atoms which may be substituted with —O(CO)—R⁴² or —(CO)O—R⁴³, an alkyl group having 1 to 18 carbon atoms which may be substituted with an aryl group, or an alkyl group having 1 to 18 carbon atoms which may be substituted with a hydroxyl group. In a case where R⁵ is an alkyl group, R⁵ may be linked to R⁴, R⁶, R¹⁰, and R¹² to form a ring which may contain an O atom. Particularly, R⁵ may be an alkyl group having 1 to 18 carbon atoms, an alkyl group having 1 to 18 carbon atoms which may be substituted with —O(CO)—R⁴² or —(CO)O—R⁴³, an alkyl group having 1 to 12 carbon atoms which may be substituted with an aryl group, or an alkyl group having 1 to 8 carbon atoms which may be substituted with a hydroxyl group, and an alkyl group having 1 to 18 carbon atoms or an alkyl group having 1 to 18 carbon atoms which may be substituted with —O(CO)—R⁴² or —(CO)O—R⁴³.

R⁷ and R⁸ may each independently represent a hydrogen atom, a hydrocarbon group having 1 to 18 carbon atoms, an alkyl group having 1 to 18 carbon atoms which may be substituted with —O(CO)—R⁴² or —(CO)O—R⁴³, an alkyl group having 1 to 8 carbon atoms which may be substituted with an aryl group, or an alkyl group having 1 to 8 carbon atoms which may be substituted with a hydroxyl group. Alternatively, it may be that R⁷ and R⁸ be linked to each other to form a 4- to 7-membered ring which may contain an O atom.

R⁵ is not linked to R⁷ or R⁸ and does not form a ring with R⁷ or R⁸.

a+b may be 1 or 2, or 1. c+d may be 1 or 2, or 1.

The compound represented by Formula (1) is a compound represented by Formula (21).

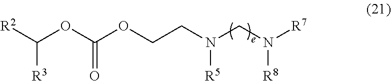

In the formula, R² and R³ each independently represent a hydrocarbon group containing one or more unsaturated bond and having 3 to 24 carbon atoms, or R² and R³ each independently represent a group represented by R³¹-L²-R³²—, or one of R² and R³ represents a group represented by R³¹-L²-R³²— and the other represents a hydrocarbon group having 3 to 24 carbon atoms, R³¹ represents a hydrocarbon group having 1 to 24 carbon atoms, L² represents —O(CO)O—, —O(CO)—, —(CO)O—, —O—, or a group represented by the following formula,

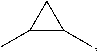

and R³² represents a divalent hydrocarbon linking group having 1 to 18 carbon atoms, R⁵ represents an alkyl group having 1 to 18 carbon atoms which may be substituted with —O(CO)—R⁴² or —(CO)O—R⁴³ where R⁴² and R⁴³ each independently represent a hydrocarbon group having 1 to 18 carbon atoms, R⁷ and R⁸ each independently represent an alkyl group having 1 to 4 carbon atoms.

In formula (21), may be one of R² and R³ is a group represented by R³¹-L²-R³²—, and the other is a hydrocarbon group having 3 to 24 carbon atoms. In formula (21), L2 may represent —O (CO)— or —(CO) O—.

In some embodiments, the ionizable lipid suitable for use in the present disclosure are ionizable lipids of formula (2) or a pharmaceutically acceptable salt thereof:

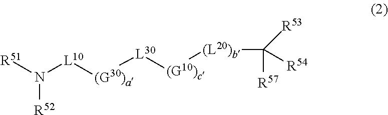

wherein
R⁵¹ and R⁵² are each independently a hydrocarbon group having 1 to 21 carbon atoms optionally substituted with R³⁵;
R³⁵ is a hydroxyl group, -G20-CH(R⁵⁵)(R⁵⁶), —N(R¹⁸)(R⁵⁹), or -G²⁰-R⁶⁰;
G²⁰ is —(CO)O— or —O(CO)—;
R⁵⁵ and R⁵⁶ are each independently hydrogen or a hydrocarbon group having 1 to 18 carbon atoms;
R⁵⁸ and R⁵⁹ are each independently hydrogen or a cyclic hydrocarbon group having 3 to 6 carbon atoms, optionally substituted with R³⁶;
R⁶⁰ is a hydrocarbon group having 1 to 18 carbon atoms;
R³⁶ is —N(R⁶¹)(R⁶²), or -G²⁰-R⁶⁵.
R⁶¹ and R⁶² are each independently hydrogen or a cyclic hydrocarbon group having 3 to 6 carbon atoms;
R⁶⁵ is a hydrocarbon group having 1 to 18 carbon atoms, -L⁴⁰-CH(R⁶⁶)(R⁶⁷), or -G²⁰-R⁶⁶.
L⁴⁰ is a divalent hydrocarbon group containing 1 to 6 carbon atoms;
R⁶⁶ and R⁶⁷ are each independently a hydrocarbon group containing 1 to 10 carbon atoms or an alkoxy group containing 1 to 10 carbon atoms;
L¹⁰ is a divalent hydrocarbon group containing 1 to 18 carbon atoms;
G³⁰ is —S(CO)N(R⁶⁴)—;
R⁶⁴ is -L³⁰-G²⁰-CH(R⁵⁵)(R⁵⁶);
a′ is 0 or 1;
G¹⁰ is G²⁰, —O(CO)O—, or —N(R⁶³)C(O)—;
c′ is 0 or 1;
R⁶³ is a hydrocarbon group containing 1 to 18 carbon atoms;
L²⁰ is a divalent hydrocarbon group containing 1 to 6 carbon atoms;
b′ is 0 or 1;
R⁵³, R⁵⁴, and R⁵⁷ are each independently hydrogen or a hydrocarbon group containing 1 to 18 carbon atoms optionally substituted with R³⁶; and
L³⁰ is a single bond or a hydrocarbon group containing 1 to 18 carbon atoms.

The compound represented by formula (2) may be represented by formula (2a)

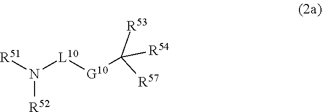

wherein
R⁵¹ and R⁵² are each independently a hydrocarbon group having 1 to 21 carbon atoms optionally substituted with R³⁵;
R³⁵ is a hydroxyl group or -G²⁰-CH(R⁵⁵)(R⁵⁶);
G²⁰ is —(CO)O— or —O(CO)—;
R⁵⁵ and R⁵⁶ are each independently hydrogen or a hydrocarbon group having 1 to 18 carbon atoms;
L¹⁰ is a divalent hydrocarbon group containing 1 to 18 carbon atoms;
G¹⁰ is —(CO)O— or —O(CO)—; and
R⁵³, R⁵⁴, and R⁵⁷ are each independently hydrogen or a hydrocarbon group containing 1 to 18 carbon atoms.

The compound represented by formula (2) may be represented by formula (2b)

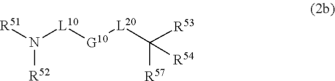

wherein
R⁵¹ and R⁵² are each independently a hydrocarbon group having 1 to 21 carbon atoms;
L¹⁰ is a divalent hydrocarbon group containing 1 to 18 carbon atoms;
G¹⁰ is —O(CO)O—;
L²¹ is a divalent hydrocarbon group containing 1 to 6 carbon atoms;
R⁵³, R⁵⁴, and R⁵⁷ are each independently hydrogen or a hydrocarbon group containing 1 to 18 carbon atoms optionally substituted with R³⁶;
R³⁶ is —O(CO)—R⁶⁵;
R⁶⁵ is a hydrocarbon group having 1 to 18 carbon atoms or -L⁴⁰-CH(R⁶⁶)(R⁶⁷);
L⁴⁰ is a divalent hydrocarbon group containing 1 to 6 carbon atoms; and
R⁶⁶ and R⁶⁷ are each independently an alkoxy group containing 1 to 10 carbon atoms.

The compound represented by formula (2) may be represented by formula (2c)

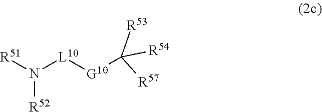

wherein
R⁵¹ and R⁵² are each independently a hydrocarbon group having 1 to 21 carbon atoms;
L¹⁰ is a divalent hydrocarbon group containing 1 to 18 carbon atoms;
G¹⁰ is —N(R⁶³)C(O)—;
R⁶³ is a hydrocarbon group containing 1 to 18 carbon atoms;
R⁵³, R⁵⁴, and R⁵⁷ are each independently hydrogen or a hydrocarbon group containing 1 to 18 carbon atoms optionally substituted with R³⁶;
R³⁶ is —(CO)OR⁶⁵;
R⁶⁵ is -L⁴⁰-CH(R⁶⁶)(R⁶⁷);
L⁴⁰ is a divalent hydrocarbon group containing 1 to 6 carbon atoms; and
R⁶⁶ and R⁶⁷ are each independently a hydrocarbon group containing 1 to 10 carbon atoms.

The compound represented by formula (2) may be represented by formula (2d)

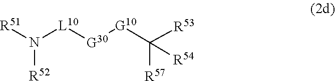

wherein
R⁵¹ and R⁵² are each independently a hydrocarbon group having 1 to 21 carbon atoms;
L¹⁰ is a divalent hydrocarbon group containing 1 to 18 carbon atoms;
G³⁰ is —S(CO)NR⁶⁴—;
R⁶⁴ is -L³⁰-G²⁰-CH(R⁵⁵)(R⁵⁶)
L³⁰ is a single bond or a hydrocarbon group containing 1 to 18 carbon atoms;
G²⁰ is —(CO)O—;
R⁵⁵ and R⁵⁶ are each independently hydrogen or a hydrocarbon group having 1 to 18 carbon atoms;
G¹⁰ is —(CO)O—; and
R⁵³, R⁵⁴, and R⁵⁷ are each independently hydrogen or a hydrocarbon group containing 1 to 18 carbon atoms.

A hydrocarbon group having 1 to 21 carbon atoms from R⁵¹ or R⁵² is preferably an alkyl group having 1 to 21 carbon atoms, an alkenyl group having 2 to 21 carbon atoms, or an alkynyl group having 2 to 21 carbon atoms, more preferably an alkyl group having 1 to 21 carbon atoms, or an alkenyl group having 2 to 21 carbon atoms. The alkyl group having 1 to 21 carbon atoms may be linear or branched, and may be chain or cyclic. The number of carbon atoms is preferably 3 to 21, and more preferably 5 to 21 carbon atoms. Examples include propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, decyl group, undecyl group, dodecyl group, tridecyl group, trimethyldodecyl group (preferably a 3,7,11-trimethyldodecyl group), tetradecyl group, pentadecyl group, hexadecyl group, heptadecyl group and octadecyl group. The alkenyl group having 2 to 18 carbon atoms may be linear or branched, and may be chain or cyclic. The number of carbon atoms is preferably 3 to 21, and more preferably 5 to 18. Examples include allyl group, prenyl group, pentanyl group, hexenyl group, heptenyl group, octenyl group, nonenyl group (preferably (Z)-2-nonenyl group or (E)-2-nonenyl group), decenyl group, undecenyl group, dodecenyl group, dodecadienyl group, tridecenyl group (preferably (Z)-trideca-8-enyl group), tetradecenyl group (preferably tetradeca-9-enyl group), pentadecenyl group (preferably (Z)-pentadeca-8-enyl group), hexadecenyl group (preferably (Z)-hexadeca-9-enyl group), hexadecadienyl group, heptadecenyl group (preferably (Z)-heptadeca-8-enyl group), heptadecadienyl group (preferably (8Z, 11Z)-heptadeca-8,11-dienyl group), octadecenyl group (preferably (Z)-octadeca-9-enyl group), octadecadienyl groups (preferably (9Z, 12Z)-octadeca-9,12-dienyl group). The alkynyl group having 2 to 21 carbon atoms may be linear or branched, and may be chain or cyclic. The number of carbon atoms is preferably 3 to 21, and more preferably 5 to 21 carbon atoms. Examples include propargyl group, butynyl group, pentynyl group, hexynyl group, heptynyl group, octynyl group, nonynyl group, decynyl group, undecynyl group, dodecynyl group, tetradecynyl group, pentadecynyl group, hexadecynyl group, heptadecynyl group, octadecynyl group and the like. Examples of hydrocarbon groups having 1 to 18 carbon atoms include those example groups specifically listed among the hydrocarbon groups having 1 to 21 carbon atoms that have 1 to 18 carbon atoms.

For a cyclic hydrocarbon group, a cycloalkyl group having 3 to 10 carbon atoms, a cycloalkenyl group having 3 to 10 carbon atoms, a cycloalkynyl group having 3 to 10 carbon atoms, or an aryl group having 6 to 10 carbon atoms are preferable.

For a hydrocarbon group having 1 to 6 carbon atoms an alkyl group having 1 to 6 carbon atoms, an alkenyl group having 2 to 6 carbon atoms or an alkynyl group having 2 to 6 carbon atoms, is preferable, and an alkyl group having 1 to 6 carbon atoms or an alkenyl group having 2 to 6 carbon atoms is more preferable. The alkyl group having 1 to 6 carbon atoms may be linear or branched, and may be a chain or cyclic. Specific examples thereof include propyl group, isopropyl group, cyclopropyl group, butyl group, isobutyl group, tert-butyl group, cyclobutyl group, pentyl group, cyclopentyl group and hexyl group. The alkenyl group having 2 to 6 carbon atoms may be linear or branched, and may be a chain or cyclic. Specific examples thereof include allyl, prenyl, pentenyl, and hexenyl. The alkynyl group having 2 to 6 carbon atoms may be linear or branched, and may be a chain or cyclic. Specific examples thereof include propargyl, butynyl, pentynyl, and hexynyl.

The hydrocarbon group having 1 to 10 carbon atoms from R⁶⁶ and R⁶⁷ is preferably an alkyl group having 1 to 10 carbon atoms, an alkenyl group having 2 to 10 carbon atoms, or an alkynyl group having 2 to 10 carbon atoms, and preferably an alkyl group having 1 to 10 carbon atoms or an alkenyl group having 2 to 10 carbon atoms. The alkyl group having 1 to 10 carbon atoms may be linear or branched, and may be chain or cyclic. The number of carbon atoms is preferably 3 to 10, and more preferably 5 to 10 carbon atoms. Specific examples include a propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, and decyl group. The alkenyl group having 2 to 10 carbon atoms may be linear or branched, and may be chain or cyclic. The number of carbon atoms is preferably 3 to 10, more preferably 5 to 10. Specific examples include allyl group, prenyl group, pentenyl group, hexenyl group, heptenyl group, octenyl group, a nonenyl group (preferably (Z)-2-nonenyl group or (E)-2-nonenyl group), and decenyl group. The alkynyl group having 2 to 10 carbon atoms may be linear or branched, and may be chain or cyclic. The number of carbon atoms is preferably 3 to 10, and more preferably 5 to 10 carbon atoms. Specific examples thereof include propargyl group, butynyl group, pentynyl group, hexynyl group, heptynyl group, octynyl group, noninyl group and a decynyl group.

The compound represented by Formula (1), (2), (2a), (2c), or (2d) may form a salt.

Examples of the salt in a basic group include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Examples of the salt in an acidic group include salts with alkali metals such as sodium and potassium; salts with alkaline earth metals such as calcium and magnesium; ammonium salts; salts with nitrogen-containing organic bases such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-p-phenethylamine, 1-ephenamine, and N,N′-dibenzylethylenediamine, and the like.

Among the above salts, for example, pharmacologically acceptable salts may be employed. In some embodiments, the lipid represented by the formula (1) and a method for producing the same are described in WO 2019/235635 A1 and WO 2021/095876 A1 (which are incorporated herein by reference in their entirety).

In some embodiments of the disclosure, the lipid represented by Formula (1) or a salt is the lipid, FL-A:

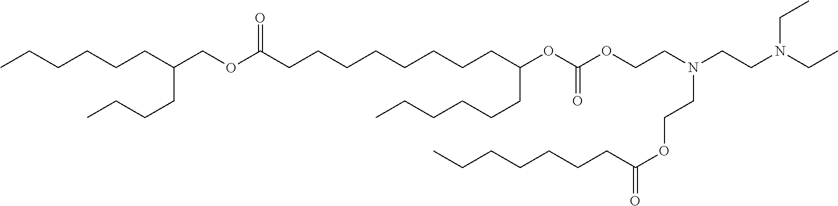 [SMILES: CCCCCCCC(=O)OCCN(CCOC(=O)OC(CCCCCC)CCCCCCCCC(=O)OCC(CCCC)CCCCCC)CCN(CC)CC]

In some embodiments of the disclosure, the ionizable lipid may be the following lipids.

MC3; ([(6Z,9Z,28Z,31Z)-heptatriaconta-6,9,28,31-tetraen-19-yl]4-(dimethylamino)butanoate) WO 2010/054405 A1:

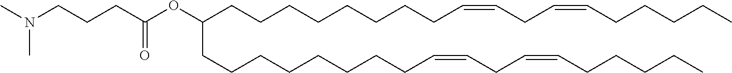 [SMILES: CCCCC/C=C\C/C=C\CCCCCCCCC(CCCCCCCC/C=C\C/C=C\CCCCC)OC(=O)CCCN(C)C]

L-319; (bis[(Z)-non-2-enyl]9-[4-(dimethylamino)butanoyloxy]heptadecanedioate) WO 2011/153493 A2, WO 2013/086354 A1, WO 2013/086322 A1:

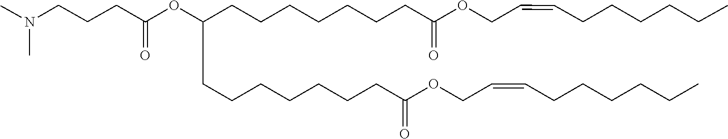 [SMILES: CCCCCC/C=C\COC(=O)CCCCCCCC(CCCCCCCC(=O)OC/C=C\CCCCCC)OC(=O)CCCN(C)C]

ALC-0315; (6-[6-(2-hexyldecanoyloxy)hexyl-(4-hydroxybutyl)amino]hexyl 2-hexyldecanoate) WO 2017/075331 A1:

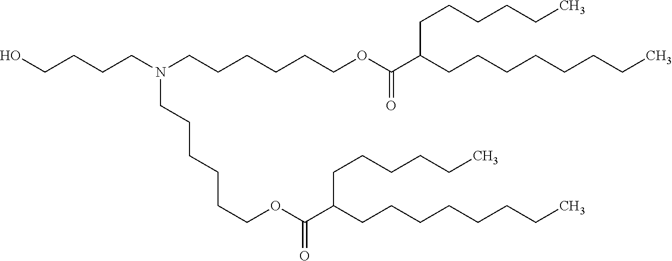 [SMILES: CCCCCCCCC(CCCCCC)C(=O)OCCCCCCN(CCCCO)CCCCCCOC(=O)C(CCCCCC)CCCCCCCC]

SM-102; (heptadecan-9-yl 8-[2-hydroxyethyl-(6-oxo-6-undecoxyhexyl)amino]octanoate) WO 2017/099823 A1:

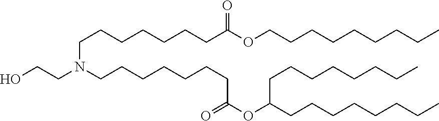 [SMILES: CCCCCCCCCOC(=O)CCCCCCCN(CCO)CCCCCCCC(=O)OC(CCCCCCCC)CCCCCCCC]

Lipid 5; (nonyl 8-[(8-heptadecan-9-yloxy-8-oxooctyl)-(2-hydroxyethyl)amino]octanoate) WO 2017/099823 A1:

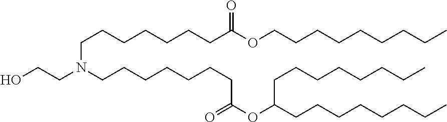 [SMILES: CCCCCCCCCOC(=O)CCCCCCCN(CCO)CCCCCCCC(=O)OC(CCCCCCCC)CCCCCCCC]

Lipid 29; (undecan-3-yl 8-[(8-heptadecan-9-yloxy-8-oxooctyl)-[3-[[2-(methylamino)-3,4-dioxocyclobuten-1-yl]amino]propyl]amino]octanoate) Adv. Funct. Mater. 2021, 2106727, DOI: 10.1002/adfm.202106727:

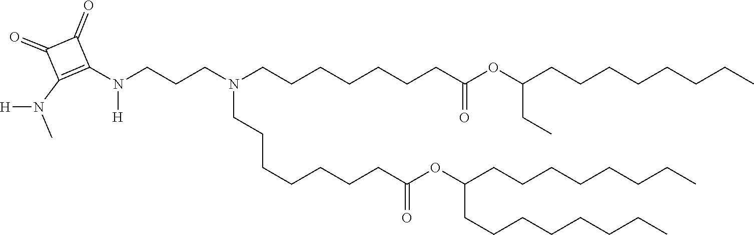 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

ATX-100; (pentadecan-8-yl 4-[3-(dimethylamino)propylsulfanylcarbonyl-(4-oxo-4-pentadecan-8-yloxybutyl)amino]butanoate) WO 2019/191780 A1:

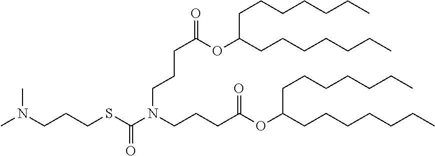 [SMILES: CCCCCCCC(CCCCCCC)OC(=O)CCCN(CCCC(=O)OC(CCCCCCC)CCCCCCC)C(=O)SCCCN(C)C]

Lipid A9; (bis(2-butyloctyl) 10-[3-(dimethylamino)propyl-nonanoylamino]nonadecanedioate) WO 2017/004143 A1:

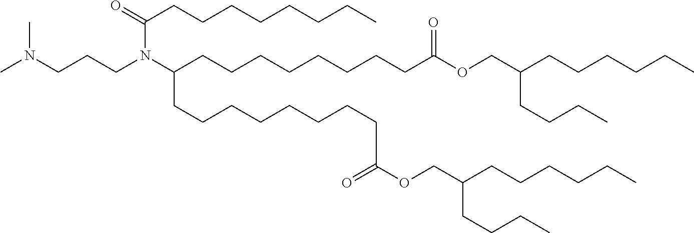 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

Lp01; ([2-[3-(diethylamino)propoxycarbonyloxymethyl]-3-(4,4-dioctoxybutanoyloxy)propyl](9Z,12Z)-octadeca-9,12-dienoate) WO 2015/09534 A2, WO 2020/219876 A1:

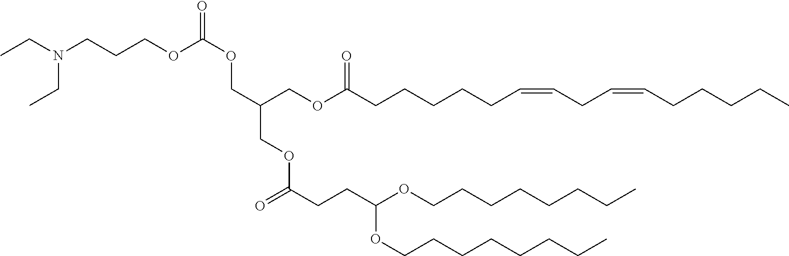 [SMILES: CCCCC/C=C\C/C=C\CCCCCC(=O)OCC(COC(=O)CCC(OCCCCCCCC)OCCCCCCCC)COC(=O)OCCCN(CC)CC]

TCL053; ([2-[4-(dimethylamino)butanoyloxymethyl]-3-[(Z)-tetradec-9-enoyl]oxy-2-[[(Z)-tetradec-9-enoyl]oxymethyl]propyl](Z)-tetradec-9-enoate) WO 2020/032184 A1:

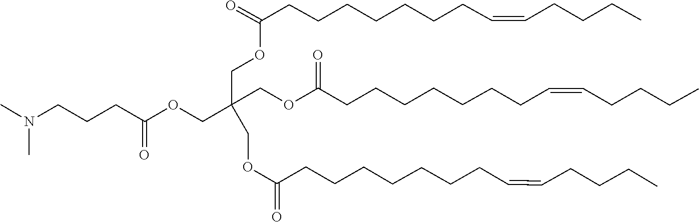 [SMILES: CCCC/C=C\CCCCCCCC(=O)OCC(COC(=O)CCCCCCC/C=C\CCCC)(COC(=O)CCCCCCC/C=C\CCCC)COC(=O)CCCN(C)C]

cKK-E12:

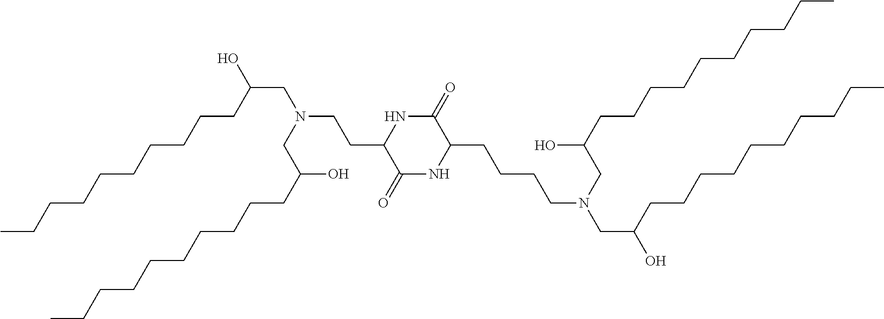 [SMILES: CCCCCCCCCCC(O)CN(CCCCC1NC(=O)C(CCN(CC(O)CCCCCCCCCC)CC(O)CCCCCCCCCC)NC1=O)CC(O)CCCCCCCCCC]

C12-200:

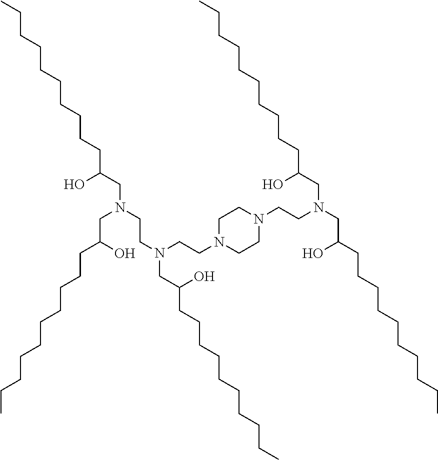 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

306Oi10:

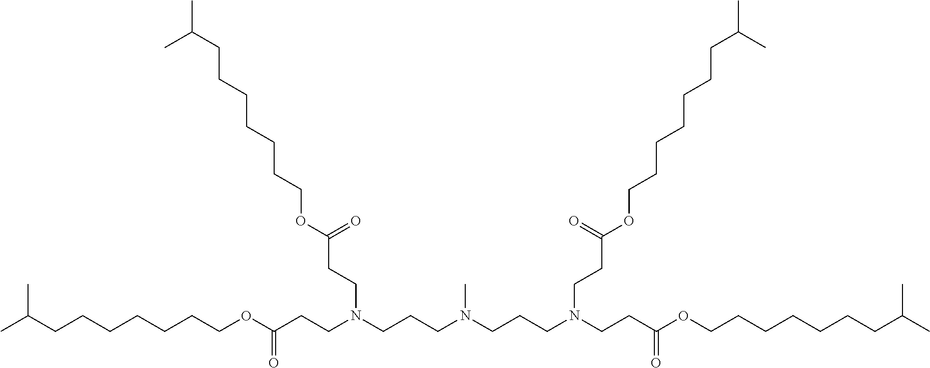 [SMILES: CC(C)CCCCCCCOC(=O)CCN(CCCN(C)CCCN(CCC(=O)OCCCCCCCC(C)C)CCC(=O)OCCCCCCCC(C)C)CCC(=O)OCCCCCCCC(C)C]

93-O17S:

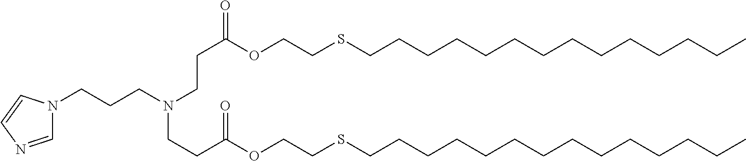 [SMILES: CCCCCCCCCCCCCCSCCOC(=O)CCN(CCCn1ccnc1)CCC(=O)OCCSCCCCCCCCCCCCCC]

YSK05:

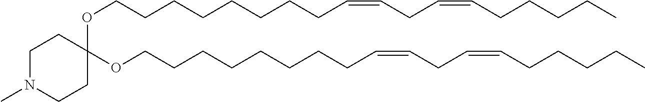 [SMILES: CCCCC/C=C\C/C=C\CCCCCCCCOC1(OCCCCCCCC/C=C\C/C=C\CCCCC)CCN(C)CC1]

Example ionizable lipids of formula (II) include:

MC3; ([(6Z,9Z,28Z,31Z)-heptatriaconta-6,9,28,31-tetraen-19-yl]4-(dimethylamino)butanoate) WO 2010/054405 A1:

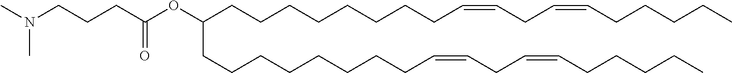 [SMILES: CCCCC/C=C\C/C=C\CCCCCCCCC(CCCCCCCC/C=C\C/C=C\CCCCC)OC(=O)CCCN(C)C]

L-319; (bis[(Z)-non-2-enyl]9-[4-(dimethylamino)butanoyloxy]heptadecanedioate)

WO 2011/153493 A2, WO 2013/086354 A1, WO 2013/086322 A1:

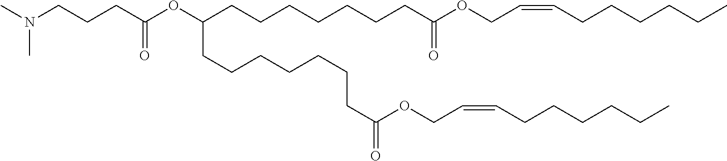 [SMILES: CCCCCC/C=C\COC(=O)CCCCCCCC(CCCCCCCC(=O)OC/C=C\CCCCCC)OC(=O)CCCN(C)C]

ALC-0315; (6-[6-(2-hexyldecanoyloxy)hexyl-(4-hydroxybutyl)amino]hexyl 2-hexyldecanoate) WO 2017/075331 A1:

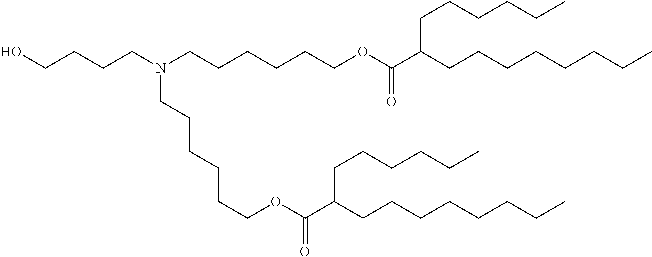 [SMILES: CCCCCCCCC(CCCCCC)C(=O)OCCCCCCN(CCCCO)CCCCCCOC(=O)C(CCCCCC)CCCCCCCC]

SM-102; (heptadecan-9-yl 8-[2-hydroxyethyl-(6-oxo-6-undecoxyhexyl)amino]octanoate) WO 2017/099823 A1:

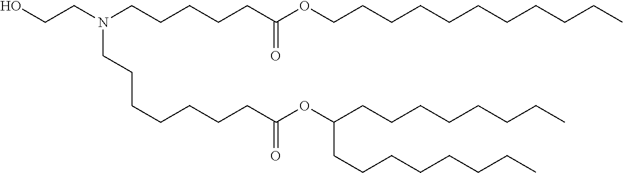 [SMILES: CCCCCCCCCCCOC(=O)CCCCCN(CCO)CCCCCCCC(=O)OC(CCCCCCCC)CCCCCCCC]

Lipid 5; (nonyl 8-[(8-heptadecan-9-yloxy-8-oxooctyl)-(2-hydroxyethyl)amino]octanoate) WO 2017/099823 A1:

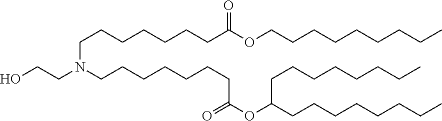 [SMILES: CCCCCCCCCOC(=O)CCCCCCCN(CCO)CCCCCCCC(=O)OC(CCCCCCCC)CCCCCCCC]

Lipid 29; (undecan-3-yl 8-[(8-heptadecan-9-yloxy-8-oxooctyl)-[3-[[2-(methylamino)-3,4-dioxocyclobuten-1-yl]amino]propyl]amino]octanoate) Adv. Funct. Mater. 2021, 2106727, DOI: 10.1002/adfm.202106727:

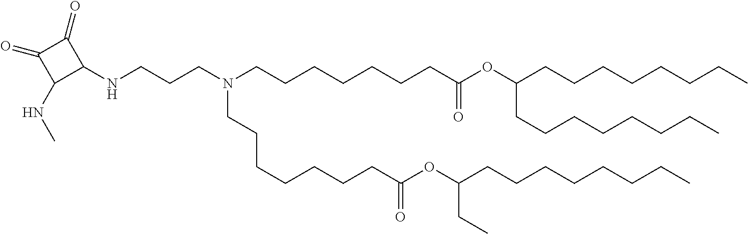 [SMILES: CCCCCCCCC(CC)OC(=O)CCCCCCCN(CCCCCCCC(=O)OC(CCCCCCCC)CCCCCCCC)CCCNC1C(=O)C(=O)C1NC]

ATX-100; (pentadecan-8-yl 4-[3-(dimethylamino)propylsulfanylcarbonyl-(4-oxo-4-pentadecan-8-yloxybutyl)amino]butanoate) WO 2019/191780 A1:

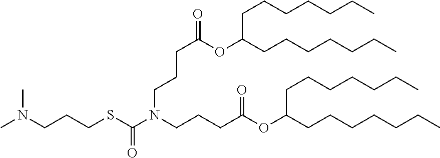 [SMILES: CCCCCCCC(CCCCCCC)OC(=O)CCCN(CCCC(=O)OC(CCCCCCC)CCCCCCC)C(=O)SCCCN(C)C]

Lipid A9; (bis(2-butyloctyl) 10-[3-(dimethylamino)propyl-nonanoylamino]nonadecanedioate) WO 2017/004143 A1:

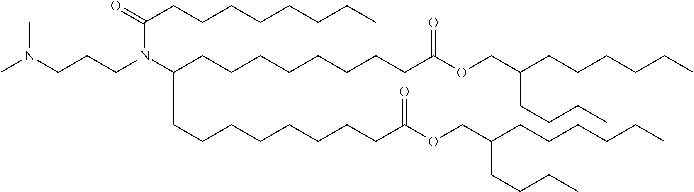 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

Lp01; ([2-[3-(diethylamino)propoxycarbonyloxymethyl]-3-(4,4-dioctoxybutanoyloxy)propyl](9Z,12Z)-octadeca-9,12-dienoate) WO 2015/09534 A2, WO 2020/219876 A1:

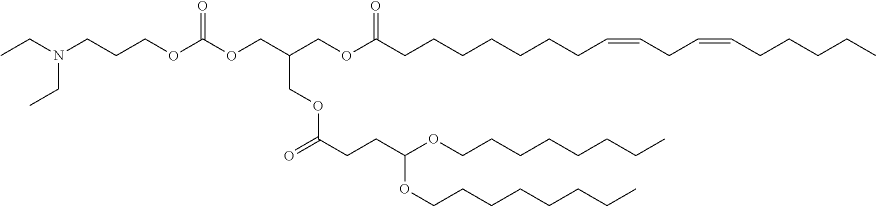 [SMILES: CCCCC/C=C\C/C=C\CCCCCCCC(=O)OCC(COC(=O)CCC(OCCCCCCCC)OCCCCCCCC)COC(=O)OCCCN(CC)CC]

TCL053; ([2-[4-(dimethylamino)butanoyloxymethyl]-3-[(Z)-tetradec-9-enoyl]oxy-2-[[(Z)-tetradec-9-enoyl]oxymethyl]propyl](Z)-tetradec-9-enoate) WO 2020/032184 A1:

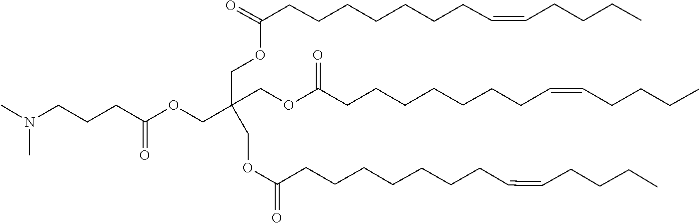 [SMILES: CCCC/C=C\CCCCCCCC(=O)OCC(COC(=O)CCCCCCC/C=C\CCCC)(COC(=O)CCCCCCC/C=C\CCCC)COC(=O)CCCN(C)C]

TCL065; ([2-[5-(dimethylamino)pentanoyloxymethyl]-3-(3-pentyloctanoyloxy)-2-(3-pentyloctanoyloxymethyl)propyl]3-pentyloctanoate) WO 2020/032184 A1:

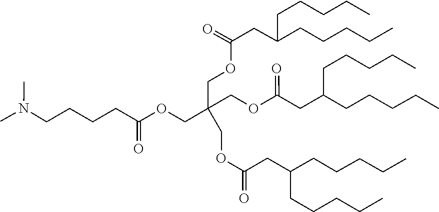 [SMILES: CCCCCC(CCCCC)CC(=O)OCC(COC(=O)CCCCN(C)C)(COC(=O)CC(CCCCC)CCCCC)COC(=O)CC(CCCCC)CCCCC]

Lipid 9; ([(6Z,16Z)-12-[6-(dimethylamino)hexanoyloxy]docosa-6,16-dien-11-yl](Z)-undec-5-enoate) WO 2021/188389 A2:

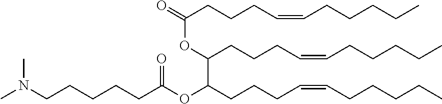 [SMILES: CCCCC/C=C\CCCC(=O)OC(CCC/C=C\CCCCC)C(CCC/C=C\CCCCC)OC(=O)CCCCCN(C)C]

Lipid 19; ([(6Z,16Z)-12-[6-(dimethylamino)hexanoyloxy]docosa-6,16-dien-11-yl](9Z,12Z)-octadeca-9,12-dienoate) WO 2021/188389 A2:

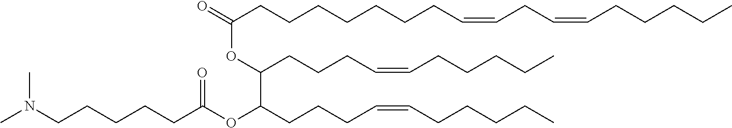 [SMILES: CCCCC/C=C\C/C=C\CCCCCCCC(=O)OC(CCC/C=C\CCCCC)C(CCC/C=C\CCCCC)OC(=O)CCCCCN(C)C]

GCL1; ([(6Z,16Z)-12-[(Z)-dec-4-enyl]docosa-6,16-dien-11-yl]5-(dimethylamino)pentanoate) WO 2020/219941 A1:

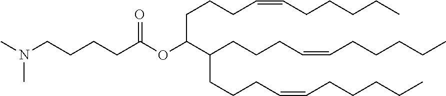 [SMILES: CCCCC/C=C\CCCC(CCC/C=C\CCCCC)C(CCC/C=C\CCCCC)OC(=O)CCCCN(C)C]

CL4H6; ([7-[4-(dipropylamino)butyl]-7-hydroxy-13-[(Z)-octadec-9-enoyl]oxytridecyl](Z)-octadec-9-enoate):

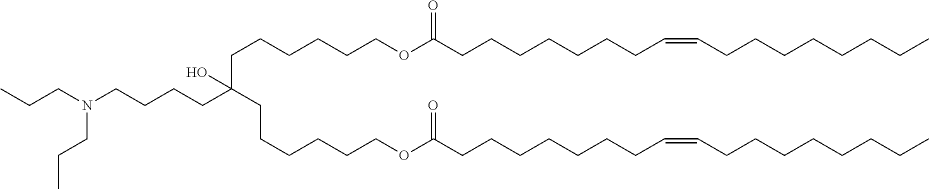 [SMILES: CCCCCCCC/C=C\CCCCCCCC(=O)OCCCCCCC(O)(CCCCCCOC(=O)CCCCCCC/C=C\CCCCCCCC)CCCCN(CCC)CCC]

### EXEMPLIFICATION

### Methods

mRNA Isolation

mRNA was isolated using Dynabeads™ mRNA DIRECT™ Purification Kit (Invitrogen #61012) according to manufacturer instructions. Briefly, cells were harvested and lysed in 100 μl of Lysis/Binding Buffer (100 mM Tris-HCl, pH 7.5, 500 mM LiCl, 10 mM EDTA, 1% Lithium dodecyl sulfate (LiDS), 5 mM dithiothreitol (DTT)). 20 μl of magnetic beads suspension was added to a PCR plate and then the lysed cells were added to the beads and mixed for 5 minutes. After removing supernatant, magnetic beads were washed 2 times with 100 μl Wash Buffer A (10 mM Tris-HCl, pH 7.5, 150 mM LiCl, 1 mM EDTA, 0.1% LiDS) and then washed 2 times with 100 μl of Wash Buffer B (10 mM Tris-HCl, pH 7.5, 150 mM LiCl, 1 mM EDTA). Next, 20 μl Elution Buffer (10 mM Tris-HCl, pH 7.5) was added and mRNA was eluted at 80° C. Beads were captured on magnetic stand and 20 μl of supernatant was transferred to another 96-well plate.

cDNA Synthesis

cDNA was synthesized using ABI High Capacity cDNA Reverse Transcription Kit (Applied Biosystems #4368814) according to manufacturer instructions. Briefly, 10 μl of a master mix containing 2 μl 10× Buffer, 0.8 μl 25×dNTPs, 2 μl 10× Random primers, 1 μl Reverse Transcriptase, and 4.2 μl of water per reaction was added to 10 μl mRNA solution that was isolated using the above protocol. Plates were sealed, mixed, and incubated on an thermal cycler for 10 minutes at room temperature, followed by 2 hours at 37° C. and 5 minutes at 85° C.

### Real Time PCR

2 μl of cDNA was added to a master mix containing 0.5 μl ACTB TaqMan Probe (Applied Biosystems #Hs99999903_m1) or 0.5 μl SIRPA TaqMan probe (Applied Biosystems #Hs00388955_m1) and 5 μl TaqMan Fast Advanced Master Mix (Applied Biosystems #4444556) per well in a 384-well plate. Real time PCR was done in a Light Cycler 480 (Roche). Each duplex was tested in two or three independent transfections, and each transfection was assayed in duplicate, unless otherwise noted.

To calculate relative fold change, real time data was analyzed using the ΔΔCt method and normalized to assays performed with cells transfected with the same concentration of siRNA against luciferase, or mock transfected cells. IC50s were calculated using a Graphpad Prism software.

### Lipid Chemical Structures

In some embodiments, the lipid chemical is FL-A or 2-butyloctyl 3-ethyl-12-hexyl-6-(2-(octanoyloxy)ethyl)-10-oxo-9,11-dioxa-3,6-diazahenicosan-21-oate described in WO2019/235635 (herein incorporated in its entirety by reference):

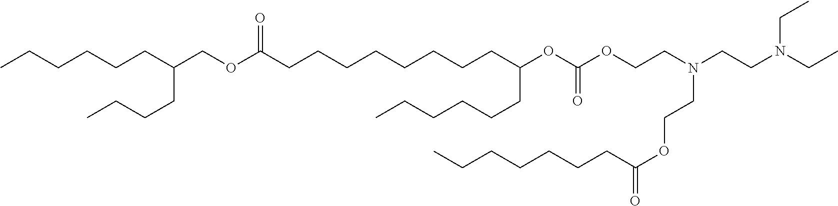 [SMILES: CCCCCCCC(=O)OCCN(CCOC(=O)OC(CCCCCC)CCCCCCCCC(=O)OCC(CCCC)CCCCCC)CCN(CC)CC]

In some embodiments, the lipid chemical is MC3 or (6Z,9Z,28Z,31Z)-heptatriaconta-6,9,28,31-tetraen-19-yl 4-(dimethylamino)butanoate described in WO2010/054405 (herein incorporated in its entirety by reference):

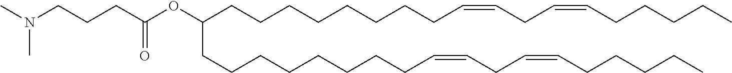 [SMILES: CCCCC/C=C\C/C=C\CCCCCCCCC(CCCCCCCC/C=C\C/C=C\CCCCC)OC(=O)CCCN(C)C]

In some embodiments, the lipid chemical is Lipid 5 or heptadecan-9-yl 8-((2-hydroxyethyl)(8-(nonyloxy)-8-oxooctyl)amino)octanoate described in WO2017/099823 (herein incorporated in its entirety by reference):

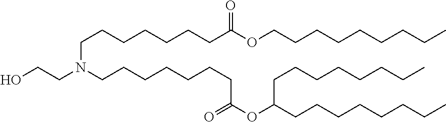 [SMILES: CCCCCCCCCOC(=O)CCCCCCCN(CCO)CCCCCCCC(=O)OC(CCCCCCCC)CCCCCCCC]

In some embodiments, the lipid chemical is distearoylphosphatidylcholine (DSPC) or 1,2-distearoyl-sn-glycero-3-phosphocholine:

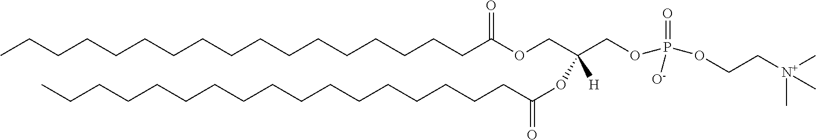 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the lipid chemical is cholesterol:

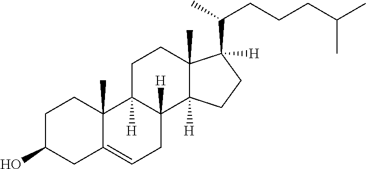

In some embodiments, the lipid chemical is 1,2-Dimyristoyl-rac-glycero-3-methoxypolyethylene glycol-2000 (DMG-PEG2000):

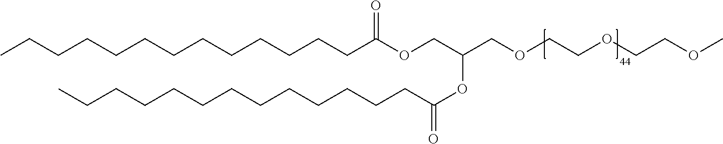

### Lipid Nanoparticle (LNP) Formulation

siRNA was diluted in 10 mM citrate buffer, pH 3.0, (aqueous phase) while the appropriate amounts of lipids were co-dissolved in 200 proof ethanol (ethanol phase). Nanoparticles formulated via microfluidic device were synthesized at a 3:1 v/v ratio of the aqueous phase to the ethanol phase. LNPs were then dialyzed against PBS in a 20 kDa MWCO cassette at 4° C. or room temperature overnight.

### Particle Size Measurement

LNP particle size and PDI (polydispersity index) were obtained using a Zetasizer (Malvern). For size measurement, LNPs were diluted in PBS at a 1/200 v/v ratio and z-average values were reported. For zeta potential measurement, LNPs were diluted in 0.1×PBS at a 1/200 v/v ratio.

Quantification of siRNA Concentration and Encapsulation

The siRNA concentration in dialyzed particles was determined via a modified Quant-iT RiboGreen RNA assay (Thermo Fisher). A nanoparticle dilution of ˜1 ng pL-1 siRNA was made in TE buffer (pH 8.5) and siRNA standards were made ranging from 2 ng pL-1 to 0.125 ng pL-1. 50 μL of each solution was added to separate wells in a 96-well black polystyrene plate. To each well was added either 50 μL of TE buffer. The plate was incubated at 37° C. for 15 minutes with shaking at 350 rpm. Following the incubation, the diluted RiboGreen reagent was added (100 pL per well), and the plate was incubated as before for 3 minutes. RiboGreen fluorescence was measured according to the supplied protocol using a Tecan plate reader, and the siRNA standard was used to determine nanoparticle siRNA concentration. It should be noted that two separate standards were made: one with and without Triton-X. The particles in TE buffer were used to determine un-encapsulated siRNA concentration and TE-TX, and encapsulation efficiency was determined via the following equation:

$$EE \% = ( 1 - \frac{R N {A}_{TE}}{R N {A}_{T X}} ) \times 100$$

### Sequence Alignment

The amino acid and mRNA sequences and were aligned using Clustal Omega.

### Example 1: siRNAs Targeting SIRPα

In certain embodiments of the disclosure, siRNA duplexes were designed to target human or mouse transcripts annotated in the NCBI Gene database (Tables 1 and 2). In further embodiments, the present disclosure contemplates the design of siRNA duplexes that target household pets, such as domesticated cat or dog transcripts annotated in the NCBI Gene database (Table 3).

TABLE 1
Human SIRPα mRNA Target Sequences

| SEQ ID NO: | NCBI Ref. No. |
|---|---|
| SEQ ID NO: 4 | NM_001040022.1 |
| SEQ ID NO: 7 | NM_001040023.2 |
| SEQ ID NO: 8 | NM_001330728.1 |
| SEQ ID NO: 9 | NM_080792.3 |

TABLE 2
Mouse SIRPα mRNA Target Sequences

| SEQ ID NO: | NCBI Ref. No. |
|---|---|
| SEQ ID NO: 10 | NM_001177647.2 |
| SEQ ID NO: 11 | NM_001291019.1 |
| SEQ ID NO: 12 | NM_001291020.1 |
| SEQ ID NO: 13 | NM_001291021.1 |
| SEQ ID NO: 14 | NM_001291022.1 |
| SEQ ID NO: 15 | NM_001355158.1 |

TABLE 3
Cat and Dog SIRPα mRNA Target Sequences

| SEQ ID NO: | NCBI Ref. No. | Species |
|---|---|---|
| SEQ ID NO: 16 | XM_045053803.1 | Felis catus (domestic cat) |
| SEQ ID NO: 17 | XM_038433331.1 | Canis lupus familiaris (domestic dog) |

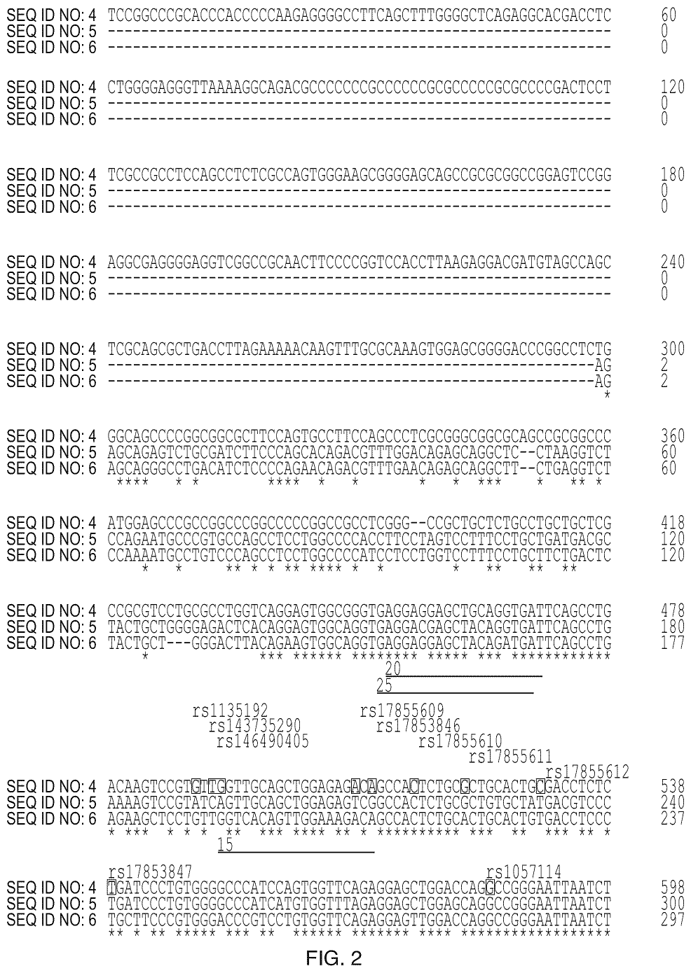

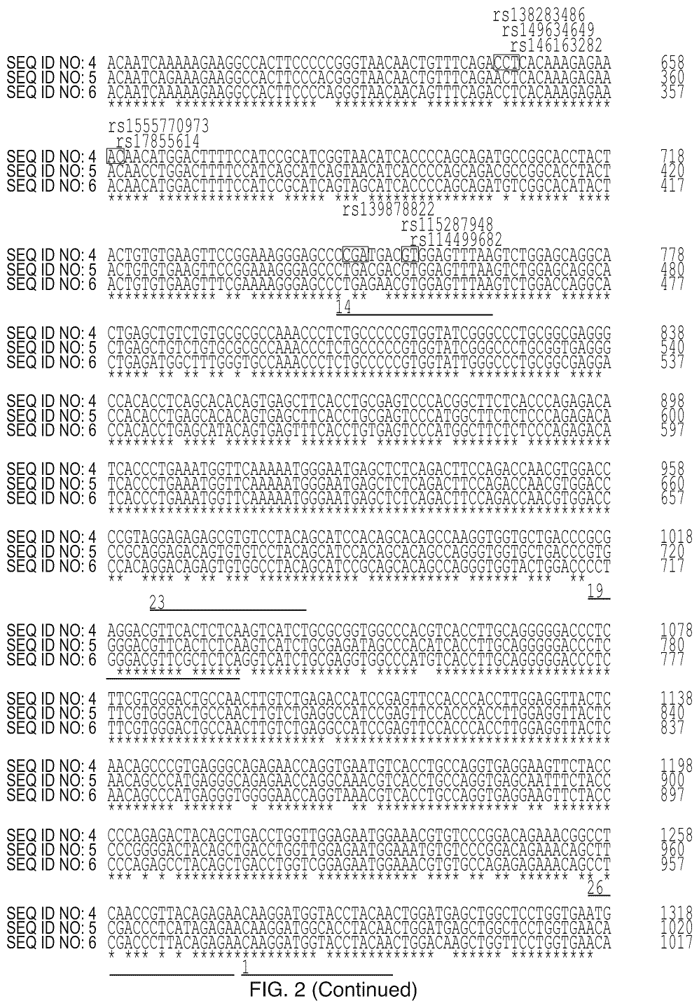

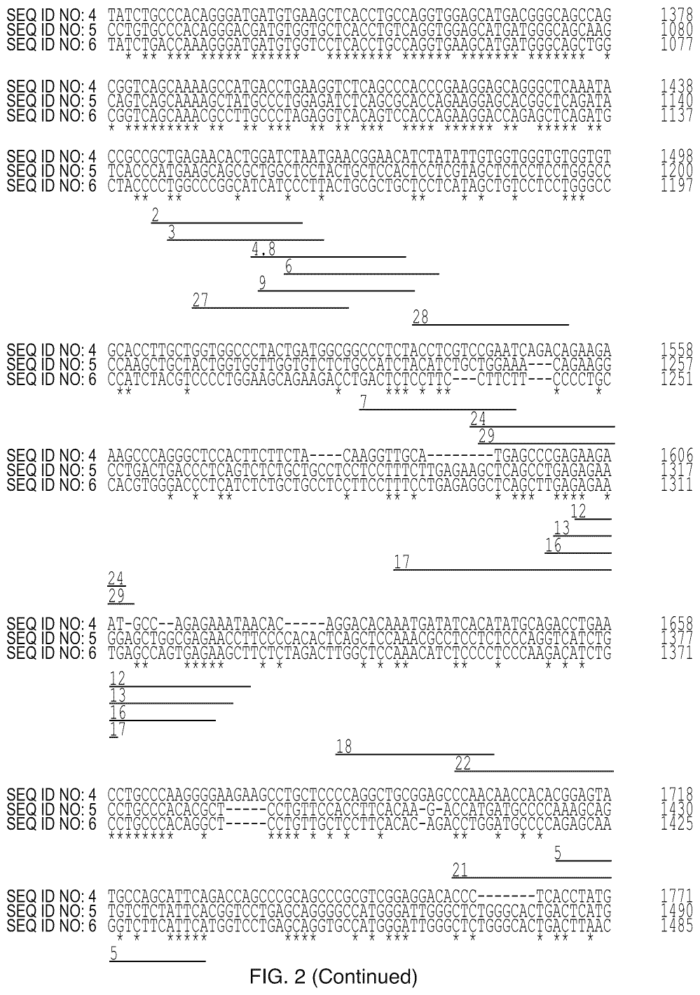

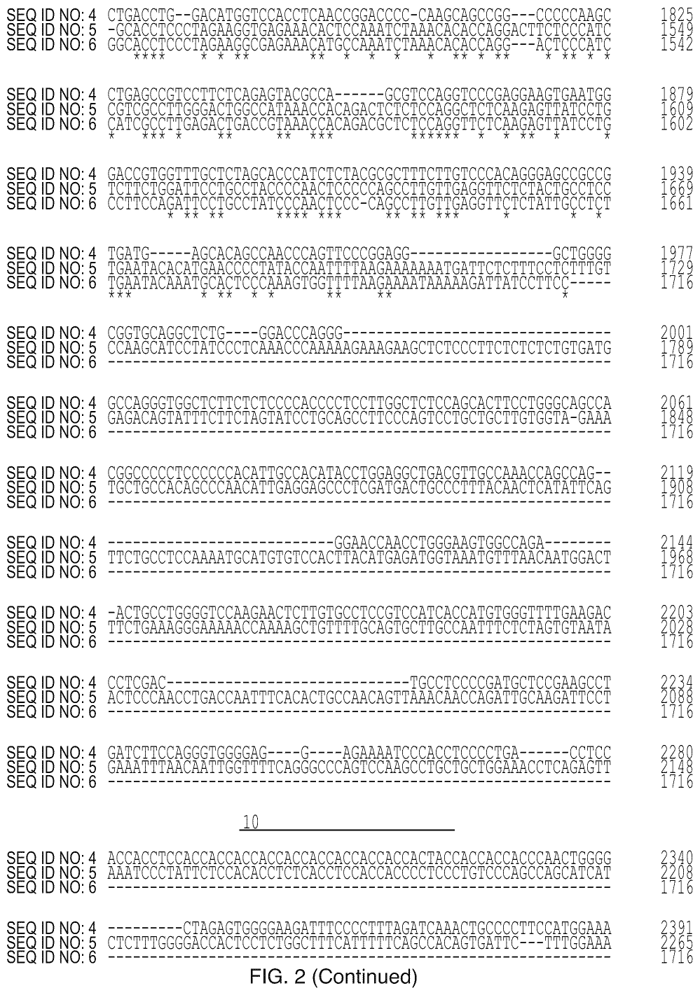

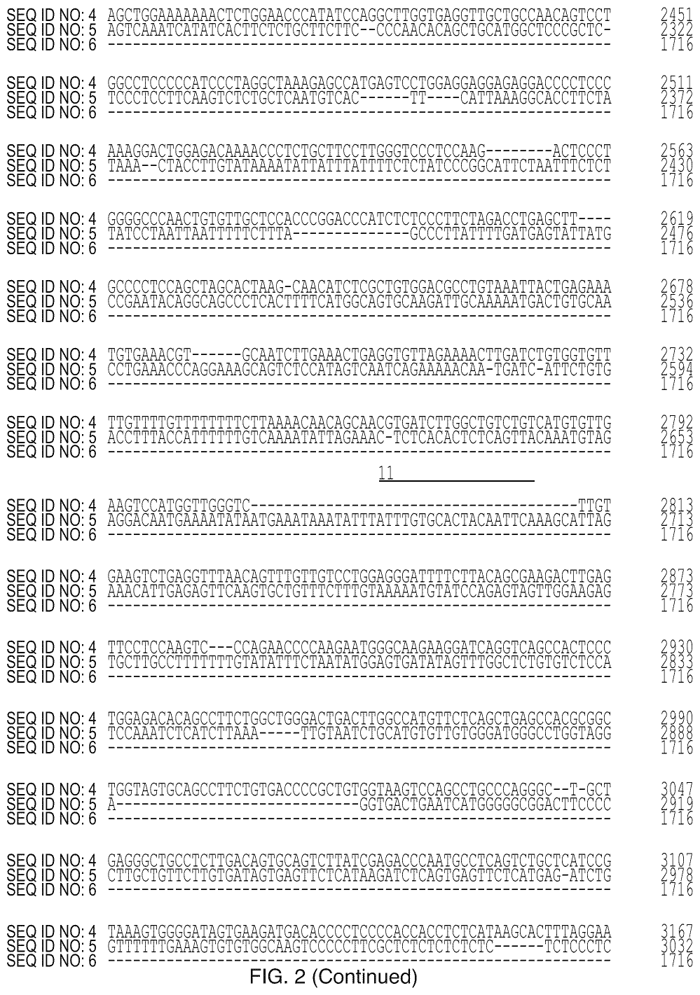

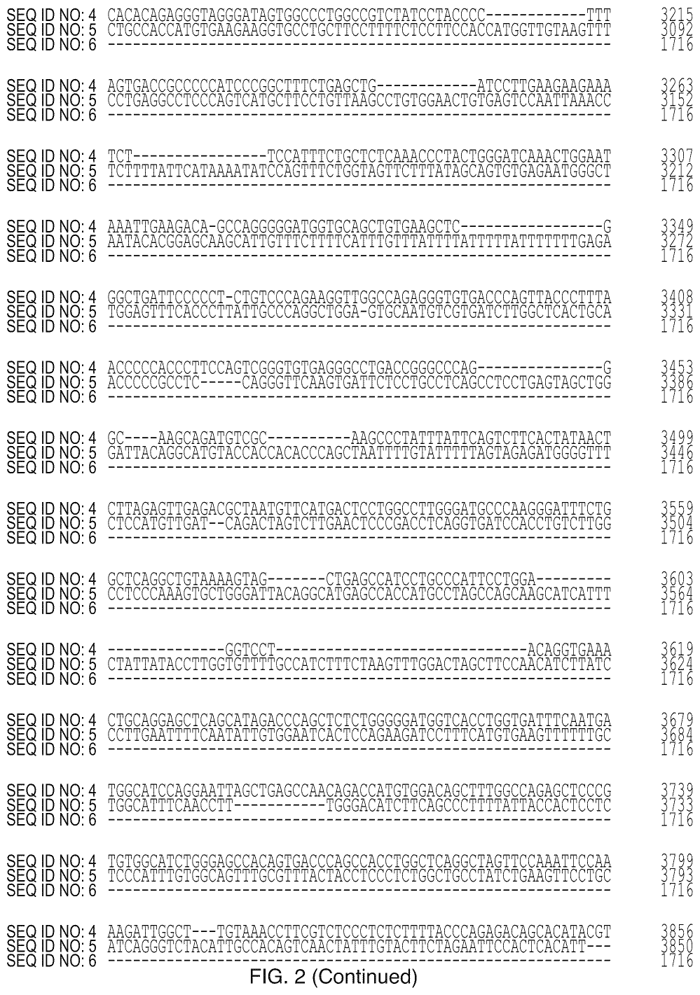

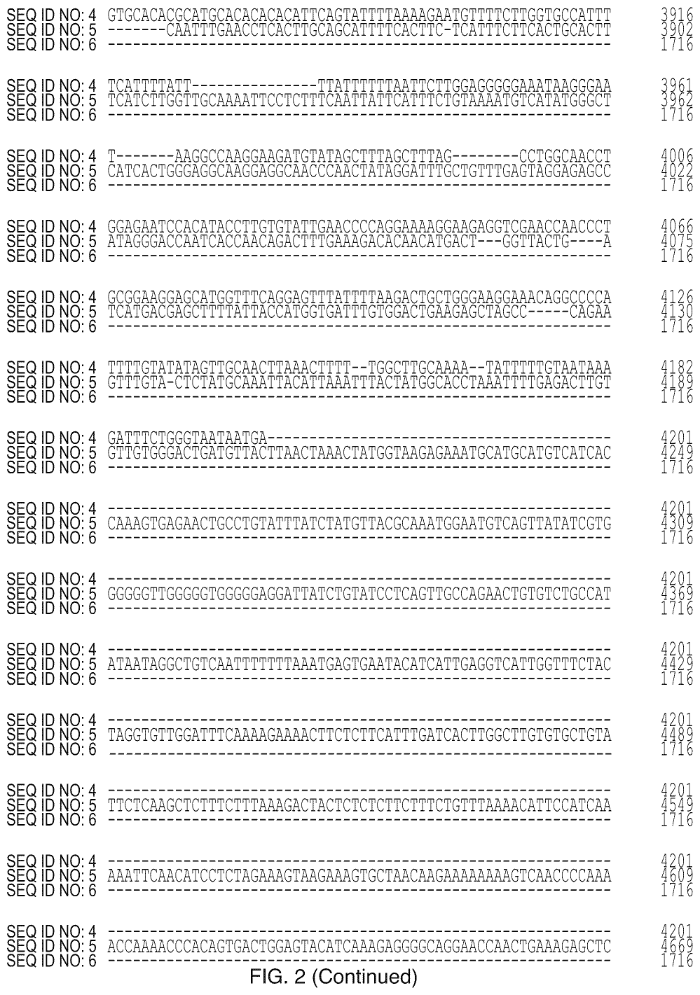

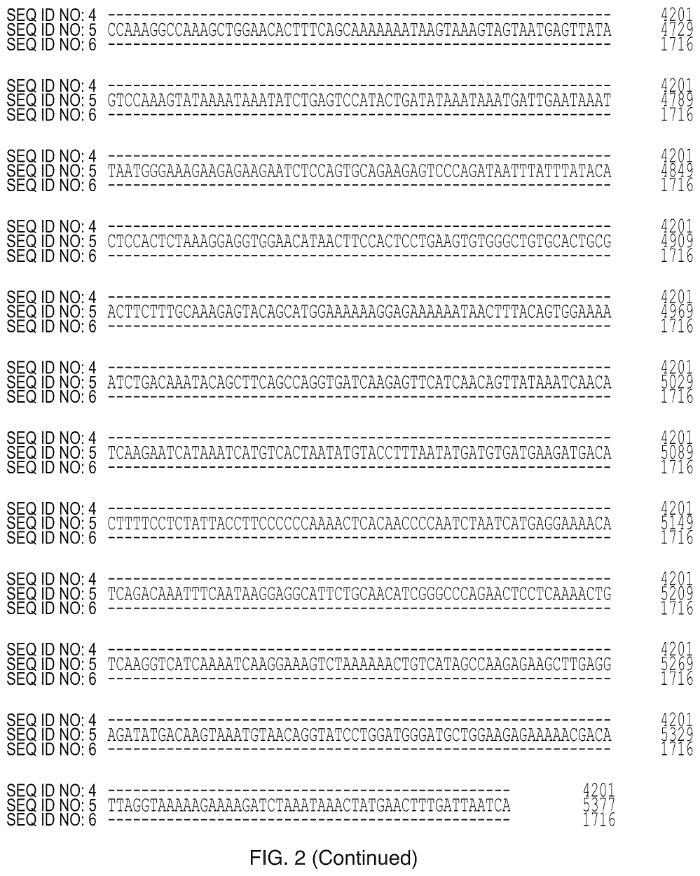

To mitigate the risk of off-target gene silencing (e.g., silencing of SIRPβ and/or SIRPγ), human SIRPα, SIRPβ and SIRPγ cDNA sequences were aligned using Clustal W2 and sequence identity was calculated (FIG. 2). Additionally, a single-nucleotide polymorphism (SNP) analysis of human SIRPα was performed based on data available at EnsEMBL (ensembl.org).

As used herein, the term “sequence identity,” refers to the extent to which two sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology). In some embodiments, codon-optimized sequences for efficient expression in different cells, tissues, and/or organisms reflect the pattern of codon usage in such cells, tissues, and/or organisms containing conservative (or non-conservative) amino acid substitutions that do not adversely affect normal activity.

Eighteen variants with global mutated allele frequency (MAF) over 0.05 were extracted (Table 4) and mapped on the SIRPα sequence (FIG. 2). Variants with minor allele frequency (MAF)>0.05 were boxed.

Human SIRPα siRNA target sequences were identified (Table 5) and mapped on the SIRPα sequence (FIG. 2; underline). Mouse SIRPα siRNA target sequences were also identified (Table 6). siRNAs with more than 75% sequence identity were predicted to have cross-reactivity. In some embodiments, siRNA sequence identity with human SIRP and SIRPγ is less than 75% to minimize off-target silencing. In some embodiments, the siRNA target sequence is located within SEQ ID NO: 4. In some embodiments, the siRNA target sequence is between 361-1875 in SEQ ID NO: 4. In some embodiments, siRNA target sequence is between 1438-1875 in SEQ ID NO: 4. In some embodiments, the siRNA target sequence is a region within SEQ ID NO:4 that is unique to SIRPα. In some embodiments, siRNA target sequence within the peri-peri-membrane, the transmembrane, and/or cytoplasmic domain of SIRPα. In some embodiments, siRNA sequence identity with cat and dog SIRPα is greater than 75% for species cross-reactivity.

TABLE 4
Human SIRPα variants

| | Global Minor | | |
|---|---|---|---|
| | allele frequency | Genomic coordinate | |
| Variant ID | (MAF) | (Chr:bp) | Alleles |
| rs1057114 | 0.4321 | 20:1915243 | G/A/C |
| rs114499682 | 0.4093 | 20:1915414 | T/C |
| rs115287948 | 0.4093 | 20:1915413 | G/A/C |
| rs139878822 | 0.3894 | 20:1915406-1915408 | CGA/— |
| rs138283486 | 0.3756 | 20:1915304 | C/A/G |
| rs146163282 | 0.3756 | 20:1915306 | T/A/C/G |
| rs149634649 | 0.3756 | 20:1915305 | C/A/T |
| rs17853847 | 0.3369 | 20:1915196 | T/C/G |
| rs1555770973 | 0.3229 | 20:1915318-1915319 | AC/G |
| rs17855612 | 0.2634 | 20:1915189 | C/A/T |
| rs1135192 | 0.2354 | 20:1915148 | G/A |
| rs143735290 | 0.2312 | 20:1915150 | T/C |
| rs146490405 | 0.231 | 20:1915151 | G/A/T |
| rs17855610 | 0.2157 | 20:1915174 | C/G/T |
| rs17855611 | 0.2153 | 20:1915180 | G/A/C |
| rs17855609 | 0.2111 | 20:1915167 | A/G/T |
| rs17853846 | 0.2109 | 20:1915169 | A/G/T |
| rs17855614 | 0.09585 | 20:1915319 | C/A |

In some embodiments, the present disclosure provides for modification of siRNA sequences. In some embodiments, the modified siRNA sequence comprises at least one nucleotide overhang covalently attached to the 3′ terminus of the sense sequence, the antisense sequence, or both the sense and the antisense sequences. In some embodiments, the modified siRNA sequence comprises a two-nucleotide overhang covalently attached to the 3′ terminus of the siRNA sense sequence, the antisense sequence, or both the sense and the antisense sequence. In some embodiments, the nucleotide overhang comprises at least one artificial nucleotide. In some embodiments, the nucleotide overhang comprises at least two artificial nucleotides. In some embodiments, the overhang is U, T, UU, TT, dT, dTdT, sdT, dTsdT, sdTsdT, or sdTdT. Non-limiting examples of modified siRNA sequences are found in Table 7 and Table 8.

In still further embodiments, the present disclosure provides for the design and use of siRNA sequences comprising one or more modified nucleotides. In some embodiments, the modified nucleotides are in the siRNA sense sequence. In some embodiments, the modified nucleotides are in the siRNA antisense sequence. In still further embodiments, the modified nucleotides are in both the siRNA sense and antisense sequences.

In some embodiments the modified nucleotide is a chemical modification. Chemical modifications may include modifications of the phosphate backbone (e.g., phosphorothioate linkages or boranophosphate linkages), ribose ring modifications such as 2′-O-methyl and/or 2′-fluoro and/or 4′-thio modifications, and locked or unlocked nucleic acids. Other modifications may include pseudouridine, 2-thiouridine, 4-thiouridine, 5-azauridine, 5-hydroxyuridine, 5-aminouridine, 5-methyluridine, 2-thiopseudouridine, 4-thiopseudouridine, 5-hydroxypseudouridine, 5-methylpseudouridine, 5-aminopseudouridine, pseudoisocytidine, 5-methylcytidine, N4-methylcytidine, 2-thiocytidine, 5-azacytidine, 5-hydroxycytidine, 5-aminocytidine, N4-methylpseudoisocytidine, 2-thiopseudoisocytidine, 5-hydroxypseudoisocytidine, 5-aminopseudoisocytidine, 5-methylpseudoisocytidine, N6-methyladenosine, 7-deazaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In some embodiments, the modified nucleotide is 2′-O-methyladenosine (mA); 2′-O-methylcytidine (mC), 2′-O-methylguanonsine (mG), or 2′-O-methyluridine (mU). Non-limiting examples of modified siRNA sequences are found in Table 7 and Table 8.

In another aspect, the present disclosure provides for the design and use of siRNA duplexes. In some embodiments, the siRNA duplex comprises SEQ ID NO: 18 and SEQ ID NO: 19. In some embodiments, the siRNA duplex comprises SEQ ID NO: 20 and SEQ ID NO: 21. In some embodiments, the siRNA duplex comprises SEQ ID NO: 22 and SEQ ID NO: 23. In some embodiments, the siRNA duplex comprises SEQ ID NO: 24 and SEQ ID NO: 25. In some embodiments, the siRNA duplex comprises SEQ ID NO: 26 and SEQ ID NO: 27. In some embodiments, the siRNA duplex comprises SEQ ID NO: 28 and SEQ ID NO: 29. In some embodiments, the siRNA duplex comprises SEQ ID NO: 30 and SEQ ID NO: 31. In some embodiments, the siRNA duplex comprises SEQ ID NO: 32 and SEQ ID NO: 33. In some embodiments, the siRNA duplex comprises SEQ ID NO: 34 and SEQ ID NO: 35. In some embodiments, the siRNA duplex comprises SEQ ID NO: 36 and SEQ ID NO: 37. In some embodiments, the siRNA duplex comprises SEQ ID NO: 38 and SEQ ID NO: 39. In some embodiments, the siRNA duplex comprises SEQ ID NO: 40 and SEQ ID NO: 41. In some embodiments, the siRNA duplex comprises SEQ ID NO: 42 and SEQ ID NO: 43. In some embodiments, the siRNA duplex comprises SEQ ID NO: 44 and SEQ ID NO: 45. In some embodiments, the siRNA duplex comprises SEQ ID NO: 46 and SEQ ID NO: 47. In some embodiments, the siRNA duplex comprises SEQ ID NO: 48 and SEQ ID NO: 49. In some embodiments, the siRNA duplex comprises SEQ ID NO: 50 and SEQ ID NO: 51. In some embodiments, the siRNA duplex comprises SEQ ID NO: 52 and SEQ ID NO: 53. In some embodiments, the siRNA duplex comprises SEQ ID NO: 54 and SEQ ID NO: 55. In some embodiments, the siRNA duplex comprises SEQ ID NO: 56 and SEQ ID NO: 57. In some embodiments, the siRNA duplex comprises SEQ ID NO: 58 and SEQ ID NO: 59. In some embodiments, the siRNA duplex comprises SEQ ID NO: 60 and SEQ ID NO: 61. In some embodiments, the siRNA duplex comprises SEQ ID NO: 62 and SEQ ID NO: 63. In some embodiments, the siRNA duplex comprises SEQ ID NO: 64 and SEQ ID NO: 65. In some embodiments, the siRNA duplex comprises SEQ ID NO: 66 and SEQ ID NO: 67. In some embodiments, the siRNA duplex comprises SEQ ID NO: 68 and SEQ ID NO: 69. In some embodiments, the siRNA duplex comprises SEQ ID NO: 70 and SEQ ID NO: 71. In some embodiments, the siRNA duplex comprises SEQ ID NO: 72 and SEQ ID NO: 73. In some embodiments, the siRNA duplex comprises SEQ ID NO: 74 and SEQ ID NO: 75. In some embodiments, the siRNA duplex comprises SEQ ID NO: 76 and SEQ ID NO: 77. In some embodiments, the siRNA duplex comprises SEQ ID NO: 78 and SEQ ID NO: 79. In some embodiments, the siRNA duplex comprises SEQ ID NO: 80 and SEQ ID NO: 81. In some embodiments, the siRNA duplex comprises SEQ ID NO: 82 and SEQ ID NO: 83. In some embodiments, the siRNA duplex comprises SEQ ID NO: 84 and SEQ ID NO: 85. In some embodiments, the siRNA duplex comprises SEQ ID NO: 86 and SEQ ID NO: 87. In some embodiments, the siRNA duplex comprises SEQ ID NO: 88 and SEQ ID NO: 89. In some embodiments, the siRNA duplex comprises SEQ ID NO: 90 and SEQ ID NO: 91. In some embodiments, the siRNA duplex comprises SEQ ID NO: 92 and SEQ ID NO: 93. In some embodiments, the siRNA duplex comprises SEQ ID NO: 94 and SEQ ID NO: 95. In some embodiments, the siRNA duplex comprises SEQ ID NO: 96 and SEQ ID NO: 97. In some embodiments, the siRNA duplex comprises SEQ ID NO: 98 and SEQ ID NO: 99. In some embodiments, the siRNA duplex comprises SEQ ID NO: 100 and SEQ ID NO: 101. In some embodiments, the siRNA duplex comprises SEQ ID NO: 102 and SEQ ID NO: 103. In some embodiments, the siRNA duplex comprises SEQ ID NO: 104 and SEQ ID NO: 105. In some embodiments, the siRNA duplex comprises SEQ ID NO: 106 and SEQ ID NO: 107. In some embodiments, the siRNA duplex comprises SEQ ID NO: 108 and SEQ ID NO: 109. In some embodiments, the siRNA duplex comprises SEQ ID NO: 110 and SEQ ID NO: 111. In some embodiments, the siRNA duplex comprises SEQ ID NO: 112 and SEQ ID NO: 113. In some embodiments, the siRNA duplex comprises SEQ ID NO: 114 and SEQ ID NO: 115. In some embodiments, the siRNA duplex comprises SEQ ID NO: 116 and SEQ ID NO: 117. In some embodiments, the siRNA duplex comprises SEQ ID NO: 118 and SEQ ID NO: 119. In some embodiments, the siRNA duplex comprises SEQ ID NO: 120 and SEQ ID NO: 121. In some embodiments, the siRNA duplex comprises SEQ ID NO: 122 and SEQ ID NO: 123. In some embodiments, the siRNA duplex comprises SEQ ID NO: 124 and SEQ ID NO: 125. In some embodiments, the siRNA duplex comprises SEQ ID NO: 126 and SEQ ID NO: 127. In some embodiments, the siRNA duplex comprises SEQ ID NO: 128 and SEQ ID NO: 129. In some embodiments, the siRNA duplex comprises SEQ ID NO: 130 and SEQ ID NO: 131. In some embodiments, the siRNA duplex comprises SEQ ID NO: 132 and SEQ ID NO: 133. In some embodiments, the siRNA duplex comprises SEQ ID NO: 134 and SEQ ID NO: 135. In some embodiments, the siRNA duplex comprises SEQ ID NO: 136 and SEQ ID NO: 137. In some embodiments, the siRNA duplex comprises SEQ ID NO: 138 and SEQ ID NO: 139. In some embodiments, the siRNA duplex comprises SEQ ID NO: 140 and SEQ ID NO: 141. In some embodiments, the siRNA duplex comprises SEQ ID NO: 142 and SEQ ID NO: 143. In some embodiments, the siRNA duplex comprises SEQ ID NO: 144 and SEQ ID NO: 145. In some embodiments, the siRNA duplex comprises SEQ ID NO: 146 and SEQ ID NO: 147. In some embodiments, the siRNA duplex comprises SEQ ID NO: 148 and SEQ ID NO: 149. In some embodiments, the siRNA duplex comprises SEQ ID NO: 150 and SEQ ID NO: 151. In some embodiments, the siRNA duplex comprises SEQ ID NO: 152 and SEQ ID NO: 153. In some embodiments, the siRNA duplex comprises SEQ ID NO: 154 and SEQ ID NO: 155. In some embodiments, the siRNA duplex comprises SEQ ID NO: 156 and SEQ ID NO: 157. In some embodiments, the siRNA duplex comprises SEQ ID NO: 158 and SEQ ID NO: 159. In some embodiments, the siRNA duplex comprises SEQ ID NO: 160 and SEQ ID NO: 161. In some embodiments, the siRNA duplex comprises SEQ ID NO: 162 and SEQ ID NO: 163. In some embodiments, the siRNA duplex comprises SEQ ID NO: 164 and SEQ ID NO: 165. In some embodiments, the siRNA duplex comprises SEQ ID NO: 166 and SEQ ID NO: 167. In some embodiments, the siRNA duplex comprises SEQ ID NO: 168 and SEQ ID NO: 169. In some embodiments, the siRNA duplex comprises SEQ ID NO: 170 and SEQ ID NO: 171. In some embodiments, the siRNA duplex comprises SEQ ID NO: 172 and SEQ ID NO: 173. In some embodiments, the siRNA duplex comprises SEQ ID NO: 174 and SEQ ID NO: 175. In some embodiments, the siRNA duplex comprises SEQ ID NO: 176 and SEQ ID NO: 177. In some embodiments, the siRNA duplex comprises SEQ ID NO: 178 and SEQ ID NO: 179. In some embodiments, the siRNA duplex comprises SEQ ID NO: 180 and SEQ ID NO: 181. In some embodiments, the siRNA duplex comprises SEQ ID NO: 182 and SEQ ID NO: 183. In some embodiments, the siRNA duplex comprises SEQ ID NO: 184 and SEQ ID NO: 185. In some embodiments, the siRNA duplex comprises SEQ ID NO: 186 and SEQ ID NO: 187. In some embodiments, the siRNA duplex comprises SEQ ID NO: 188 and SEQ ID NO: 189. In some embodiments, the siRNA duplex comprises SEQ ID NO: 190 and SEQ ID NO: 191. In some embodiments, the siRNA duplex comprises SEQ ID NO: 192 and SEQ ID NO: 193. In some embodiments, the siRNA duplex comprises SEQ ID NO: 194 and SEQ ID NO: 195. In some embodiments, the siRNA duplex comprises SEQ ID NO: 196 and SEQ ID NO: 197. In some embodiments, the siRNA duplex comprises SEQ ID NO: 198 and SEQ ID NO: 199. In some embodiments, the siRNA duplex comprises SEQ ID NO: 200 and SEQ ID NO: 201. In some embodiments, the siRNA duplex comprises SEQ ID NO: 202 and SEQ ID NO: 203. In some embodiments, the siRNA duplex comprises SEQ ID NO: 204 and SEQ ID NO: 205. In some embodiments, the siRNA duplex comprises SEQ ID NO: 206 and SEQ ID NO: 207. In some embodiments, the siRNA duplex comprises SEQ ID NO: 208 and SEQ ID NO: 209. In some embodiments, the siRNA duplex comprises SEQ ID NO: 210 and SEQ ID NO: 211. In some embodiments, the siRNA duplex comprises SEQ ID NO: 212 and SEQ ID NO: 213. In some embodiments, the siRNA duplex comprises SEQ ID NO: 214 and SEQ ID NO: 215. In some embodiments, the siRNA duplex comprises SEQ ID NO: 216 and SEQ ID NO: 217. In some embodiments, the siRNA duplex comprises SEQ ID NO: 218 and SEQ ID NO: 219. In some embodiments, the siRNA duplex comprises SEQ ID NO: 220 and SEQ ID NO: 221. In some embodiments, the siRNA duplex comprises SEQ ID NO: 222 and SEQ ID NO: 223. In some embodiments, the siRNA duplex comprises SEQ ID NO: 224 and SEQ ID NO: 225. In some embodiments, the siRNA duplex comprises SEQ ID NO: 226 and SEQ ID NO: 227.

TABLE 5
Human SIRPα 19-mer mRNA Target Sequences

| Target | | | | Sequence Identity | | | |
|---|---|---|---|---|---|---|---|
| sequence | Sense | Antisense | | Human | Human | Cat | Dog |
| ID | sequence | sequence | SNPs | SIRPβ | SIRPγ | SIRPα | SIRPα |
| Target ID | CAACCGUUAC | UUGUUCUCUG | 0 | 95% | 100% | 84% | 84% |
| 1 | AGAGAACAA | UAACGGUUG | | | | | |
| | (SEQ ID NO: 18) | (SEQ ID NO: 19) | | | | | |
| Target ID | GCUGAGAACA | UAGAUCCAGU | 0 | 32% | 37% | n/a | n/a |
| 2 | CUGGAUCUA | GUUCUCAGC | | | | | |
| | (SEQ ID NO: 20) | (SEQ ID NO: 21) | | | | | |
| Target ID | UGAGAACACU | AUUAGAUCCA | 0 | 32% | 37% | n/a | n/a |
| 3 | GGAUCUAAU | GUGUUCUCA | | | | | |
| | (SEQ ID NO: 22) | (SEQ ID NO: 23) | | | | | |
| Target ID | GGAUCUAAUG | UGUUCCGUUC | 0 | 16% | 11% | n/a | n/a |
| 4 | AACGGAACA | AUUAGAUCC | | | | | |
| | (SEQ ID NO: 24) | (SEQ ID NO: 25) | | | | | |
| Target ID | CGGAGUAUGC | UGAAUGCUGG | 0 | 42% | 47% | 89% | 89% |
| 5 | CAGCAUUCA | CAUACUCCG | | | | | |
| | (SEQ ID NO: 26) | (SEQ ID NO: 27) | | | | | |
| Target ID | CUAAUGAACG | UAGAUGUUCC | 0 | 32% | 26% | 61% | 61% |
| 6 | GAACAUCUA | GUUCAUUAG | | | | | |
| | (SEQ ID NO: 28) | (SEQ ID NO: 29) | | | | | |
| Target ID | CCCUCUACCU | AUUCGGACGA | 0 | 53% | n/a | 95% | 95% |
| 7 | CGUCCGAAU | GGUAGAGGG | | | | | |
| | (SEQ ID NO: 30) | (SEQ ID NO: 31) | | | | | |
| Target ID | GGAUCUAAUG | UGUUCCGUUC | 0 | 16% | 11% | n/a | n/a |
| 8 | AACGGAACA | AUUAGAUCC | | | | | |
| | (SEQ ID NO: 32) | (SEQ ID NO: 33) | | | | | |
| Target ID | GAUCUAAUGA | AUGUUCCGUU | 0 | 21% | 16% | n/a | n/a |
| 9 | ACGGAACAU | CAUUAGAUC | | | | | |
| | (SEQ ID NO: 34) | (SEQ ID NO: 35) | | | | | |
| Target ID | GGAGGAGAAA | AGGUGGGAUU | 0 | n/a | n/a | 61% | 61% |
| 10 | AUCCCACCU | UUCUCCUCC | | | | | |
| | (SEQ ID NO: 36) | (SEQ ID NO: 37) | | | | | |
| Target ID | CGUGAUCUUG | ACAGACAGCC | 0 | n/a | n/a | 79% | 84% |
| 11 | GCUGUCUGU | AAGAUCACG | | | | | |
| | (SEQ ID NO: 38) | (SEQ ID NO: 39) | | | | | |
| Target ID | GAAGAAUGCC | UAUUUCUCUG | 0 | n/a | n/a | 89% | 89% |
| 12 | AGAGAAAUA | GCAUUCUUC | | | | | |
| | (SEQ ID NO: 40) | (SEQ ID NO: 41) | | | | | |
| Target ID | GAGAAGAAUG | UUUCUCUGGC | 0 | n/a | n/a | 89% | 89% |
| 13 | CCAGAGAAA | AUUCUUCUC | | | | | |
| | (SEQ ID NO: 42) | (SEQ ID NO: 43) | | | | | |
| Target ID | CCGAUGACGU | UUAAACUCCA | 3 | 89% | 84% | n/a | n/a |
| 14 | GGAGUUUAA | CGUCAUCGG | | | | | |
| | (SEQ ID NO: 44) | (SEQ ID NO: 45) | | | | | |
| Target ID | GGUUGCAGCU | UGUCUCUCCA | 3 | 84% | 84% | 68% | 84% |
| 15 | GGAGAGACA | GCUGCAACC | | | | | |
| | (SEQ ID NO: 46) | (SEQ ID NO: 47) | | | | | |
| Target ID | CGAGAAGAAU | UUCUCUGGCA | 0 | n/a | n/a | 89% | 89% |
| 16 | GCCAGAGAA | UUCUUCUCG | | | | | |
| | (SEQ ID NO: 48) | (SEQ ID NO: 49) | | | | | |
| Target ID | UGCAUGAGCC | UUCUUCUCGG | 0 | n/a | n/a | 100% | 100% |
| 17 | CGAGAAGAA | GCUCAUGCA | | | | | |
| | (SEQ ID NO: 50) | (SEQ ID NO: 51) | | | | | |
| Target ID | GGACACAAAU | UGUGAUAUCA | 0 | 32% | 53% | 79% | 79% |
| 18 | GAUAUCACA | UUUGUGUCC | | | | | |
| | (SEQ ID NO: 52) | (SEQ ID NO: 53) | | | | | |
| Target ID | GCGAGGACGU | UGAGAGUGAA | 0 | 89% | 79% | 74% | 74% |
| 19 | UCACUCUCA | CGUCCUCGC | | | | | |
| | (SEQ ID NO: 54) | (SEQ ID NO: 55) | | | | | |
| Target ID | AGGAGGAGCU | AUCACCUGCA | 0 | 89% | 89% | 84% | 84% |
| 20 | GCAGGUGAU | GCUCCUCCU | | | | | |
| | (SEQ ID NO: 56) | (SEQ ID NO: 57) | | | | | |
| Target ID | CCAACAACCA | UACUCCGUGU | 0 | 32% | 32% | 95% | 95% |
| 21 | CACGGAGUA | GGUUGUUGG | | | | | |
| | (SEQ ID NO: 58) | (SEQ ID NO: 59) | | | | | |
| Target ID | UCACAUAUGC | UUCAGGUCUG | 0 | 32% | 47% | 89% | 89% |
| 22 | AGACCUGAA | CAUAUGUGA | | | | | |
| | (SEQ ID NO: 60) | (SEQ ID NO: 61) | | | | | |
| Target ID | GGAGAGAGCG | UGUAGGACAC | 0 | 89% | 84% | 79% | 74% |
| 23 | UGUCCUACA | GCUCUCUCC | | | | | |
| | (SEQ ID NO: 62) | (SEQ ID NO: 63) | | | | | |
| Target ID | CCGAAUCAGA | UUUCUUCUGU | 0 | n/a | n/a | 95% | 95% |
| 24 | CAGAAGAAA | CUGAUUCGG | | | | | |
| | (SEQ ID NO: 64) | (SEQ ID NO: 65) | | | | | |
| Target ID | GAGGAGGAGC | UCACCUGCAG | 0 | 89% | 89% | 84% | 84% |
| 25 | UGCAGGUGA | CUCCUCCUC | | | | | |
| | (SEQ ID NO: 66) | (SEQ ID NO: 67) | | | | | |
| Target ID | CCUCAACCGU | UUCUCUGUAA | 0 | 79% | 89% | n/a | n/a |
| 26 | UACAGAGAA | CGGUUGAGG | | | | | |
| | (SEQ ID NO: 68) | (SEQ ID NO: 69) | | | | | |
| Target ID | GAACACUGGA | UUCAUUAGAU | 0 | 16% | 26% | n/a | n/a |
| 27 | UCUAAUGAA | CCAGUGUUC | | | | | |
| | (SEQ ID NO: 70) | (SEQ ID NO: 71) | | | | | |
| Target ID | UCUAUAUUGU | ACACCCACCA | 0 | 32% | 37% | 84% | 84% |
| 28 | GGUGGGUGU | CAAUAUAGA | | | | | |
| | (SEQ ID NO: 72) | (SEQ ID NO: 73) | | | | | |
| Target ID | CGAAUCAGAC | CUUUCUUCUG | 0 | n/a | n/a | 95% | 95% |
| 29 | AGAAGAAAG | UCUGAUUCG | | | | | |
| | (SEQ ID NO: 74) | (SEQ ID NO: 75) | | | | | |
| Luciferase | CUUACGCUGA | UCGAAGUACU | | | | | |
| | GUACUUCGA | CAGCGUAAG | | | | | |
| | (SEQ ID NO: 76) | (SEQ ID NO: 77) | | | | | |

n/a means either sequence has insertion or deletion (no identity or 0%)

TABLE 6
Mouse SIRPα 19-mer mRNA Target Sequences

| | | | Sequence | |
|---|---|---|---|---|
| Target | | | identity | |
| sequence | | | Cat | Dog |
| ID | Sense sequence | Antisense sequence | SIRPα | SIRPα |
| Target | CCGGAUCAAACAGAAGA | UUUCUUCUGUUUGAUCCGG | 84% | 84% |
| ID_m1 | AA | (SEQ ID NO: 79) | | |
| | (SEQ ID NO: 78) | | | |
| Target | GAAGAACGCCAGGGAAA | UAUUUCCCUGGCGUUCUUC | 84% | 84% |
| ID_m2 | UA | (SEQ ID NO: 81) | | |
| | (SEQ ID NO: 80) | | | |
| Target | AGACCUGAAUCUGCCCA | UUUGGGCAGAUUCAGGUCU | 84% | 84% |
| ID_m3 | AA | (SEQ ID NO: 83) | | |
| | (SEQ ID NO: 82) | | | |
| Target | GCCUAACAACCACACAG | UUCUGUGUGGUUGUUAGG | 95% | 95% |
| ID_m4 | AA | C | | |
| | (SEQ ID NO: 84) | (SEQ ID NO: 85) | | |
| Target | GUGCCUAGGCCAGAGGA | UAUCCUCUGGCCUAGGCAC | 63% | 63% |
| ID_m5 | UA | (SEQ ID NO: 87) | | |
| | (SEQ ID NO: 86) | | | |
| Target | AAGCCUGAGCCAUCUUU | AGAAAGAUGGCUCAGGCUU | 84% | 84% |
| ID_m6 | CU | (SEQ ID NO: 89) | | |
| | (SEQ ID NO: 88) | | | |
| Target | GCCAUCUUUCUCAGAGU | AUACUCUGAGAAAGAUGGC | 79% | 79% |
| ID_m7 | AU | (SEQ ID NO: 91) | | |
| | (SEQ ID NO: 90) | | | |
| Target | UCCGGAUCAAACAGAAG | UUCUUCUGUUUGAUCCGGA | 84% | 84% |
| ID_m8 | AA | (SEQ ID NO: 93) | | |
| | (SEQ ID NO: 92) | | | |
| Target | AAGAACGCCAGGGAAAU | UUAUUUCCCUGGCGUUCUU | 84% | 84% |
| ID_m9 | AA | (SEQ ID NO: 95) | | |
| | (SEQ ID NO: 94) | | | |
| Target | CCAUCUUUCUCAGAGUA | CAUACUCUGAGAAAGAUGG | 79% | 79% |
| ID_m10 | UG | (SEQ ID NO: 97) | | |
| | (SEQ ID NO: 96) | | | |
| Target | ACAUCAACGACAUCACA | UAUGUGAUGUCGUUGAUG | n/a | n/a |
| ID_m11 | UA | U | | |
| | (SEQ ID NO: 98) | (SEQ ID NO: 99) | | |
| Target | AGAGGAUACCCUCACCU | AUAGGUGAGGGUAUCCUCU | 84% | 84% |
| ID_m12 | AU | (SEQ ID NO: 101) | | |
| | (SEQ ID NO: 100) | | | |
| Target | UCACAUACGCAGACCUG | UUCAGGUCUGCGUAUGUGA | 95% | 84% |
| ID_m13 | AA | (SEQ ID NO: 103) | | |
| | (SEQ ID NO: 102) | | | |
| Target | GAACCAGGCCCUGUUCU | UAAGAACAGGGCCUGGUUC | 63% | 63% |
| ID_m14 | UA | (SEQ ID NO: 105) | | |
| | (SEQ ID NO: 104) | | | |
| Target | CUCUACCCAACUUGAGC | AAGCUCAAGUUGGGUAGA | 68% | 68% |
| ID_m15 | UU | G | | |
| | (SEQ ID NO: 106) | (SEQ ID NO: 107) | | |
| Target | CUGGAAUCCAAGAAGUG | AACACUUCUUGGAUUCCAG | n/a | 47% |
| ID_m16 | UU | (SEQ ID NO: 109) | | |
| | (SEQ ID NO: 108) | | | |
| Target | GAGAAGAACGCCAGGGA | UUUCCCUGGCGUUCUUCUC | 84% | 84% |
| ID_m17 | AA | (SEQ ID NO: 111) | | |
| | (SEQ ID NO: 110) | | | |
| Target | CCUAACAACCACACAGA | AUUCUGUGUGGUUGUUAG | 95% | 95% |
| ID_m18 | AU | G | | |
| | (SEQ ID NO: 112) | (SEQ ID NO: 113) | | |
| Target | GUGUCCAGGUCCAGAGG | UUCCUCUGGACCUGGACAC | 95% | 95% |
| ID_m19 | AA | (SEQ ID NO: 115) | | |
| | (SEQ ID NO: 114) | | | |
| Target | AAGCAUUGAGACAGGCA | UUUGCCUGUCUCAAUGCUU | 68% | 68% |
| ID_m20 | AA | (SEQ ID NO: 117) | | |
| | (SEQ ID NO: 116) | | | |
| Target | CUAACAACCACACAGAA | UAUUCUGUGUGGUUGUUA | 95% | 95% |
| ID_m21 | UA | G | | |
| | (SEQ ID NO: 118) | (SEQ ID NO: 119) | | |
| Target | UGCACGAGCCCGAGAAG | UUCUUCUCGGGCUCGUGCA | 95% | 95% |
| ID_m22 | AA | (SEQ ID NO: 121) | | |
| | (SEQ ID NO: 120) | | | |
| Target | CAAAUGACAUCAACGAC | AUGUCGUUGAUGUCAUUU | n/a | n/a |
| ID_m23 | AU | G | | |
| | (SEQ ID NO: 122) | (SEQ ID NO: 123) | | |
| Target | CGAGAAGAACGCCAGGG | UUCCCUGGCGUUCUUCUCG | 84% | 84% |
| ID_m24 | AA | (SEQ ID NO: 125) | | |
| | (SEQ ID NO: 124) | | | |
| Target | ACAAAUGACAUCAACGA | UGUCGUUGAUGUCAUUUG | n/a | n/a |
| ID_m25 | CA | U | | |
| | (SEQ ID NO: 126) | (SEQ ID NO: 127) | | |
| Target | GAUAAUAAUGCUACCCA | UGUGGGUAGCAUUAUUAU | 26% | 37% |
| ID_m26 | CA | C | | |
| | (SEQ ID NO: 128) | (SEQ ID NO: 129) | | |
| Target | AGAAGAACGCCAGGGAA | AUUUCCCUGGCGUUCUUCU | 84% | 84% |
| ID_m27 | AU | (SEQ ID NO: 131) | | |
| | (SEQ ID NO: 130) | | | |
| Target | UAACAACCACACAGAAU | AUAUUCUGUGUGGUUGUU | 95% | 95% |
| ID_m28 | AU | A | | |
| | (SEQ ID NO: 132) | (SEQ ID NO: 133) | | |
| Target | GGAUCAAACAGAAGAAA | GCUUUCUUCUGUUUGAUCC | 84% | 84% |
| ID_m29 | GC | (SEQ ID NO: 135) | | |
| | (SEQ ID NO: 134) | | | |
| Target | UCAAACAGAAGAAAGCC | UUGGCUUUCUUCUGUUUGA | 89% | 89% |
| ID_m30 | AA | (SEQ ID NO: 137) | | |
| | (SEQ ID NO: 136) | | | |
| Target | ACACAAAUGACAUCAAC | UCGUUGAUGUCAUUUGUG | n/a | n/a |
| ID_m31 | GA | U | | |
| | (SEQ ID NO: 138) | (SEQ ID NO: 139) | | |

n/a means sequence identity cannot be calculated due to insertions or deletions in either sequence

TABLE 7
Human SIRPα siRNA duplexes and sequences

| siRNA duplex ID | Sense sequence | Antisense sequence |
|---|---|---|
| sihSIRPα_1 | (SEQ ID NO: 18)-dTdT | (SEQ ID NO: 19)-dTdT |
| sihSIRPα_2 | (SEQ ID NO: 20)-dTdT | (SEQ ID NO: 21)-dTdT |
| sihSIRPα_3 | (SEQ ID NO: 22)-dTdT | (SEQ ID NO: 23)-dTdT |
| sihSIRPα_4 | (SEQ ID NO: 24)-dTdT | (SEQ ID NO: 25)-dTdT |
| sihSIRPα_5 | (SEQ ID NO: 26)-dTdT | (SEQ ID NO: 27)-dTdT |
| sihSIRPα_6 | (SEQ ID NO: 28)-dTdT | (SEQ ID NO: 29)-dTdT |
| sihSIRPα_7 | (SEQ ID NO: 30)-dTdT | (SEQ ID NO: 31)-dTdT |
| sihSIRPα_8 | (SEQ ID NO: 32)-dTdT | (SEQ ID NO: 33)-dTdT |
| sihSIRPα_9 | (SEQ ID NO: 34)-dTdT | (SEQ ID NO: 35)-dTdT |
| sihSIRPα_10 | (SEQ ID NO: 36)-dTdT | (SEQ ID NO: 37)-dTdT |
| sihSIRPα_11 | (SEQ ID NO: 38)-dTdT | (SEQ ID NO: 39)-dTdT |
| sihSIRPα_12 | (SEQ ID NO: 40)-dTdT | (SEQ ID NO: 41)-dTdT |
| sihSIRPα_13 | (SEQ ID NO: 42)-dTdT | (SEQ ID NO: 43)-dTdT |
| sihSIRPα_14 | (SEQ ID NO: 44)-dTdT | (SEQ ID NO: 45)-dTdT |
| sihSIRPα_15 | (SEQ ID NO: 46)-dTdT | (SEQ ID NO: 47)-dTdT |
| sihSIRPα_16 | (SEQ ID NO: 48)-dTdT | (SEQ ID NO: 49)-dTdT |
| sihSIRPα_17 | (SEQ ID NO: 50)-dTdT | (SEQ ID NO: 51)-dTdT |
| sihSIRPα_18 | (SEQ ID NO: 52)-dTdT | (SEQ ID NO: 53)-dTdT |
| sihSIRPα_19 | (SEQ ID NO: 54)-dTdT | (SEQ ID NO: 55)-dTdT |
| sihSIRPα_20 | (SEQ ID NO: 56)-dTdT | (SEQ ID NO: 57)-dTdT |
| sihSIRPα_21 | (SEQ ID NO: 58)-dTdT | (SEQ ID NO: 59)-dTdT |
| sihSIRPα_22 | (SEQ ID NO: 60)-dTdT | (SEQ ID NO: 61)-dTdT |
| sihSIRPα_23 | (SEQ ID NO: 62)-dTdT | (SEQ ID NO: 63)-dTdT |
| sihSIRPα_24 | (SEQ ID NO: 64)-dTdT | (SEQ ID NO: 65)-dTdT |
| sihSIRPα_25 | (SEQ ID NO: 66)-dTdT | (SEQ ID NO: 67)-dTdT |
| sihSIRPα_26 | (SEQ ID NO: 68)-dTdT | (SEQ ID NO: 69)-dTdT |
| sihSIRPα_27 | (SEQ ID NO: 70)-dTdT | (SEQ ID NO: 71)-dTdT |
| sihSIRPα_28 | (SEQ ID NO: 72)-dTdT | (SEQ ID NO: 73)-dTdT |
| sihSIRPα_29 | (SEQ ID NO: 74)-dTdT | (SEQ ID NO: 75)-dTdT |
| sihSIRPα_1-PM | mCAAmCmCGmUmUAmCAGA | UUGUUCUCUGmUAACGGUU |
| | GAAmCAA (SEQ ID NO: 140)- | G (SEQ ID NO: 141)-dTsdT |
| | dTsdT | |
| sihSIRPα_2-PM | GmCmUGAGAAmCAmCmUGG | mUAGAUCmCAGUGUUCUmC |
| | AmUmCmUA (SEQ ID NO: 142)- | AGC (SEQ ID NO: 143)-dTsdT |
| | dTsdT | |
| sihSIRPα_3-PM | mUGAGAAmCAmCmUGGAmU | AUmUAGAUCmCAGUGUUCU |
| | mCmUAAmU (SEQ ID NO: 144)- | mCA (SEQ ID NO: 145)-dTsdT |
| | dTsdT | |
| sihSIRPα_4-PM | GGAmUmCmUAAmUGAAmCG | UGUUCCGUUmCAUmUAGAU |
| | GAAmCA (SEQ ID NO: 146)- | CC (SEQ ID NO: 147)-dTsdT |
| | dTsdT | |
| sihSIRPα_7-PM | mCmCmCmUmCmUAmCmCmU | AUUCGGACGAGGmUAGAGG |
| | mCGmUmCmCGAAmU (SEQ ID | G (SEQ ID NO: 149)-dTsdT |
| | NO: 148)-dTsdT | |
| sihSIRPα_8-PM | GGAmUmCmUAAmUGAAmCG | UGUUCCGUUmCAUmUAGAU |
| | GAAmCA (SEQ ID NO: 150)- | CC (SEQ ID NO: 151)-dTsdT |
| | dTsdT | |
| sihSIRPα_12-PM | GAAGAAmUGmCmCAGAGAA | mUAUUUCUCUGGmCAUUCU |
| | AmUA (SEQ ID NO: 152)- | UC (SEQ ID NO: 153)-dTsdT |
| | dTsdT | |
| sihSIRPα_13-PM | GAGAAGAAmUGmCmCAGAG | UUUCUCUGGmCAUUCUUCU |
| | AAA (SEQ ID NO: 154)-dTsdT | C (SEQ ID NO: 155)-dTsdT |
| sihSIRPα_16-PM | mCGAGAAGAAmUGmCmCAG | UUCUCUGGmCAUUCUUCUC |
| | AGAA (SEQ ID NO: 156)- | G (SEQ ID NO: 157)-dTsdT |
| | dTsdT | |
| sihSIRPα_17-PM | mUGmCAmUGAGmCmCmCGA | UUCUUCUCGGGCUmCAUGm |
| | GAAGAA (SEQ ID NO: 158)- | CA (SEQ ID NO: 159)-dTsdT |
| | dTsdT | |
| sihSIRPα_18-PM | GGAmCAmCAAAmUGAmUAm | UGUGAmUAUmCAUUUGUG |
| | UmCAmCA (SEQ ID NO: 160)- | UCC (SEQ ID NO: 161)-dTsdT |
| | dTsdT | |
| sihSIRPα_19-PM | GmCGAGGAmCGmUmUmCAm | UGAGAGUGAACGUCCUCGC |
| | CmUmCmUmCA (SEQ ID NO: | (SEQ ID NO: 163)-dTsdT |
| | 162)-dTsdT | |
| sihSIRPα_20-PM | AGGAGGAGmCmUGmCAGGm | AUmCACCUGmCAGCUCCUC |
| | UGAmU (SEQ ID NO: 164)- | CU (SEQ ID NO: 165)-dTsdT |
| | dTsdT | |
| sihSIRPα_21-PM | mCmCAAmCAAmCmCAmCAm | mUACUCCGUGUGGUUGUUG |
| | CGGAGmUA (SEQ ID NO: 166)- | G (SEQ ID NO: 167)-dTsdT |
| | dTsdT | |
| sihSIRPα_22-PM | mUmCAmCAmUAmUGmCAGA | UUmCAGGUCUGmCAmUAUG |
| | mCmCmUGAA (SEQ ID NO: | UGA (SEQ ID NO: 169)-dTsdT |
| | 168)-dTsdT | |
| sihSIRPα_23-PM | GGAGAGAGmCGmUGmUmCm | UGmUAGGAmCACGCUCUCU |
| | CmUAmCA | CC (SEQ ID NO: 171)-dTsdT |
| | (SEQ ID NO: 170)-dTsdT | |
| sihSIRPα_24-PM | mCmCGAAmUmCAGAmCAGA | UUUCUUCmUGUCmUGAUUC |
| | AGAAA (SEQ ID NO: 172)- | GG (SEQ ID NO: 173)-dTsdT |
| | dTsdT | |
| sihSIRPα_25-PM | GAGGAGGAGmCmUGmCAGG | UmCACCUGmCAGCUCCUCC |
| | mUGA (SEQ ID NO: 174)- | UC (SEQ ID NO: 175)-dTsdT |
| | dTsdT | |
| sihSIRPα_26-PM | mCmCmUmCAAmCmCGmUmU | UUCUCUGmUAACGGUUGAG |
| | AmCAGAGAA (SEQ ID NO: | G (SEQ ID NO: 177)-dTsdT |
| | 176)-dTsdT | |
| sihSIRPα_27-PM | GAAmCAmCmUGGAmUmCmU | UUmCAUmUAGAUCmCAGUG |
| | AAmUGAA (SEQ ID NO: 178)- | UUC (SEQ ID NO: 179)-dTsdT |
| | dTsdT | |
| sihSIRPα_28-PM | mUmCmUAmUAmUmUGmUGG | AmCACCmCACmCAmCAAmU |
| | mUGGGmUGmU (SEQ ID NO: | AmUAGA (SEQ ID NO: 181)- |
| | 180)-dTsdT | dTsdT |
| sihSIRPα_29-PM | mCGAAmUmCAGAmCAGAAG | CUUUCUUCmUGUCmUGAUm |
| | AAAG (SEQ ID NO: 182)- | UCG (SEQ ID NO: 183)-dTsdT |
| | dTsdT | |
| siLuc | mCmUmUAmCGmCmUGAGmU | UCGAAGmUACUmCAGCGmU |
| | AmCmUmUmCGA (SEQ ID NO: | AAG (SEQ ID NO: 185)-dTsdT |
| | 184)-dTsdT | |

TABLE 8
Mouse SIRPα siRNA duplexes and sequences

| SIRNA | | |
|---|---|---|
| duplex ID | Sense sequence | Antisense sequence |
| simSIRPα_1 | (SEQ ID NO: 78)-dTdT | (SEQ ID NO: 79)-dTdT |
| simSIRPα_2 | (SEQ ID NO: 80)-dTdT | (SEQ ID NO: 81)-dTdT |
| simSIRPα_3 | (SEQ ID NO: 82)-dTdT | (SEQ ID NO: 83)-dTdT |
| simSIRPα_4 | (SEQ ID NO: 84)-dTdT | (SEQ ID NO: 85)-dTdT |
| simSIRPα_5 | (SEQ ID NO: 86)-dTdT | (SEQ ID NO: 87)-dTdT |
| simSIRPα_6 | (SEQ ID NO: 88)-dTdT | (SEQ ID NO: 89)-dTdT |
| simSIRPα_7 | (SEQ ID NO: 90)-dTdT | (SEQ ID NO: 91)-dTdT |
| simSIRPα_8 | (SEQ ID NO: 92)-dTdT | (SEQ ID NO: 93)-dTdT |
| simSIRPα_9 | (SEQ ID NO: 94)-dTdT | (SEQ ID NO: 95)-dTdT |
| simSIRPα_10 | (SEQ ID NO: 96)-dTdT | (SEQ ID NO: 97)-dTdT |
| simSIRPα_11 | (SEQ ID NO: 98)-dTdT | (SEQ ID NO: 99)-dTdT |
| simSIRPα_12 | (SEQ ID NO: 100)-dTdT | (SEQ ID NO:101)-dTdT |
| simSIRPα_13 | (SEQ ID NO: 102)-dTdT | (SEQ ID NO:103)-dTdT |
| simSIRPα 14 | (SEQ ID NO: 104)-dTdT | (SEQ ID NO: 105)-dTdT |
| simSIRPα_15 | (SEQ ID NO: 106)-dTdT | (SEQ ID NO: 107)-dTdT |
| simSIRPα_16 | (SEQ ID NO: 108)-dTdT | (SEQ ID NO: 109)-dTdT |
| simSIRPα_17 | (SEQ ID NO: 110)-dTdT | (SEQ ID NO: 111)-dTdT |
| simSIRPα_18 | (SEQ ID NO: 112)-dTdT | (SEQ ID NO: 113)-dTdT |
| simSIRPα_19 | (SEQ ID NO: 114)-dTdT | (SEQ ID NO: 115)-dTdT |
| simSIRPα_20 | (SEQ ID NO: 116)-dTdT | (SEQ ID NO: 117)-dTdT |
| simSIRPα_21 | (SEQ ID NO: 118)-dTdT | (SEQ ID NO: 119)-dTdT |
| simSIRPα_22 | (SEQ ID NO: 120)-dTdT | (SEQ ID NO: 121)-dTdT |
| simSIRPα_23 | (SEQ ID NO: 122)-dTdT | (SEQ ID NO: 123)-dTdT |
| simSIRPα_24 | (SEQ ID NO: 124)-dTdT | (SEQ ID NO: 125)-dTdT |
| simSIRPα_25 | (SEQ ID NO: 126)-dTdT | (SEQ ID NO: 127)-dTdT |
| simSIRPα_26 | (SEQ ID NO: 128)-dTdT | (SEQ ID NO: 129)-dTdT |
| simSIRPα_27 | (SEQ ID NO: 130)-dTdT | (SEQ ID NO: 131)-dTdT |
| simSIRPα_28 | (SEQ ID NO: 132)-dTdT | (SEQ ID NO: 133)-dTdT |
| simSIRPα_29 | (SEQ ID NO: 134)-dTdT | (SEQ ID NO: 135)-dTdT |
| simSIRPα_30 | (SEQ ID NO: 136)-dTdT | (SEQ ID NO: 137)-dTdT |
| simSIRPα_31 | (SEQ ID NO: 138)-dTdT | (SEQ ID NO: 139)-dTdT |
| simSIRPα_3- | AGAmCmCmUGAAmUmCmUGmCm | UUUGGGmCAGAUUmCAGGUC |
| PM | CmCAAA (SEQ ID NO: 186)-dTsdT | U (SEQ ID NO: 187)-dTsdT |
| simSIRPα_5- | GmUGmCmCmUAGGmCmCAGAGG | mUAUCCUCUGGCCmUAGGmC |
| PM | AmUA (SEQ ID NO: 188)-dTsdT | AC (SEQ ID NO: 189)-dTsdT |
| simSIRPα_10- | mCmCAmUmCmUmUmUmCmUmCA | mCAmUACUCUGAGAAAGAUG |
| PM | GAGmUAmUG (SEQ ID NO: 190)- | G (SEQ ID NO: 191)-dTsdT |
| | dTsdT | |
| simSIRPα_11- | AmCAmUmCAAmCGAmCAmUmCA | mUAUGUGAUGUCGUUGAUGU |
| PM | mCAmUA (SEQ ID NO: 192)-dTsdT | (SEQ ID NO: 193)-dTsdT |
| simSIRPα_12- | AGAGGAmUAmCmCmCmUmCAmC | AmUAGGUGAGGGmUAUCCUC |
| PM | mCmUAmU (SEQ ID NO: 194)- | U (SEQ ID NO: 195)-dTsdT |
| | dTsdT | |
| simSIRPα_13- | mUmCAmCAmUAmCGmCAGAmCm | UUmCAGGUCUGCGmUAUGUG |
| PM | CmUGAA (SEQ ID NO: 196)-dTsdT | A (SEQ ID NO: 197)-dTsdT |
| simSIRPα_17- | GAGAAGAAmCGmCmCAGGGAAA | UUUCCCmUGGCGUUCUUCUC |
| PM | (SEQ ID NO: 198)-dTsdT | (SEQ ID NO: 199)-dTsdT |
| simSIRPα_18- | mCmCmUAAmCAAmCmCAmCAmC | AUUCUGUGUGGUUGUmUAGG |
| PM | AGAAmU (SEQ ID NO: 200)-dTsdT | (SEQ ID NO: 201)-dTsdT |
| simSIRPα_19- | GmUGmUmCmCAGGmUmCmCAGA | UUCCUCUGGACCUGGAmCAC |
| PM | GGAA (SEQ ID NO: 202)-dTsdT | (SEQ ID NO: 203)-dTsdT |
| simSIRPα_20- | AAGmCAmCmCGAGAmCAGGmCA | UUUGCCUGUCUmCAAUGCUU |
| PM | AA (SEQ ID NO: 204)-dTsdT | (SEQ ID NO: 205)-dTsdT |
| simSIRPα_21- | mCmUAAmCAAmCmCAmCAmCAG | mUAUUCUGUGUGGUUGUmUA |
| PM | AAmUA (SEQ ID NO: 206)-dTsdT | G (SEQ ID NO: 207)-dTsdT |
| simSIRPα_22- | mUGmCAmCGAGmCmCmCGAGAA | UUCUUCUCGGGCUCGUGmCA |
| PM | GAA (SEQ ID NO: 208)-dTsdT | (SEQ ID NO: 209)-dTsdT |
| simSIRPα_23- | mCAAAmUGAmCAmUmCAAmCGA | AUGUCGUUGAUGUmCAUUUG |
| PM | mCAmU (SEQ ID NO: 210)-dTsdT | (SEQ ID NO: 211)-dTsdT |
| simSIRPα_24- | mCGAGAAGAAmCGmCmCAGGGA | UUCCCUGGCGUUCUUCUCG |
| PM | A (SEQ ID NO: 212)-dTsdT | (SEQ ID NO: 213)-dTsdT |
| simSIRPα_25- | AmCAAAmUGAmCAmUmCAAmCG | UGUCGUUGAUGUmCAUUUGU |
| PM | AmCA (SEQ ID NO: 214)-dTsdT | (SEQ ID NO: 215)-dTsdT |
| simSIRPα_26- | GAmUAAmUAAmUGmCmUAmCmC | UGUGGGmUAGmCAUmUAUmU |
| PM | mCAmCA (SEQ ID NO: 216)-dTsdT | AUC (SEQ ID NO: 217)-dTsdT |
| simSIRPα_27- | AGAAGAAmCGmCmCAGGGAAAm | AUUUCCCUGGCGUUCUUCU |
| PM | U (SEQ ID NO: 218)-dTsdT | (SEQ ID NO: 219)-dTsdT |
| simSIRPα_28- | mUAAmCAAmCmCAmCAmCAGAA | AmUAUUCUGUGUGGUUGUmU |
| PM | mUAmU (SEQ ID NO: 220)-dTsdT | A (SEQ ID NO: 221)-dTsdT |
| simSIRPα_29- | GGAmUmCAAAmCAGAAGAAAGm | GCUUUCUUCUGUUUGAUCC |
| PM | C (SEQ ID NO: 222)-dTsdT | (SEQ ID NO: 223)-dTsdT |
| simSIRPα_30- | UmCAAAmCAGAAGAAAGmCmCA | UUGGCUUUCUUCUGUUUGA |
| PM | A (SEQ ID NO: 224)-dTsdT | (SEQ ID NO: 225)-dTsdT |
| simSIRPα_31- | AmCAmCAAAmUGAmCAmUmCAA | UCGUUGAUGUmCAUUUGUGU |
| PM | mCGA (SEQ ID NO: 226)-dTsdT | (SEQ ID NO: 227)-dTsdT |

TABLE 9
Comparison of murine SIRPα 19-mer mRNA
Target Sequences

| Target | | |
|---|---|---|
| sequence ID | Sense sequence | Antisense sequence |
| Target ID | CCAACAACCACACGGAGU | UACUCCGUGUGGUUGUUG |
| 21 (human) | A (SEQ ID NO: 58) | G (SEQ ID NO: 59) |
| Target ID | CUAACAACCACACAGAAU | UAUUCUGUGUGGUUGUUA |
| 21 (mouse) | A (SEQ ID NO: 118) | G (SEQ ID NO: 119) |

### Example 2: Dual Response Screening of Human SIRPα siRNA Duplexes and Variants

siRNA modification variants derived from siRNA duplexes with high efficiency and specificity to human SIRPα RNA knock-down were designed according to sequence and chemical modifications (as described in Example 1) and the resulting duplex variants were further tested in THP-1-derived macrophages.

THP-1 monocytes were cultured using the standard culture condition and transferred to 96-well plates at a density of 10,000-15,000 cells per well. THP-1 monocytes were differentiated into macrophages by an incubation with 100 ng/mL phorbol 12-myristate 13-acetate (PMA) for 24 hours and subsequent recovery period for 24 hours. THP-1 macrophages were transfected with SIRPα siRNA duplexes using Viromer Blue (0.5 μl/well). A total of 30 siRNA duplexes, including variants from the original modified siRNA duplexes, were transfected into THP-1 macrophagescells and further validated. The SIRPα siRNAs were transfected at a final concentration of 5 nM and 25 nM, respectively. An anti fLuc siRNA duplex was used as a negative control. After incubating for 48 hours, total mRNA was extracted and purified, and cDNA was synthesized by reverse transcription. SIRPα expression level was quantified by real time PCR. The information and sequences of these siRNA duplexes are included in Table 10 and Table 11.

TABLE 10
Relative fold change of SIRPα mRNA level

| siRNA duplex ID | 25 nM | 5 nM |
|---|---|---|
| sihSIRPα_1 | 0.09 | 0.15 |
| sihSIRPα_2 | 0.08 | 0.14 |
| sihSIRPα_3 | 0.02 | 0.09 |
| sihSIRPα_4 | 0.03 | 0.09 |
| sihSIRPα_5 | 0.16 | 0.48 |
| sihSIRPα_6 | 0.11 | 0.18 |
| sihSIRPα_7 | 0.05 | 0.16 |
| sihSIRPα_8 | 0.08 | 0.09 |
| sihSIRPα_9 | 0.11 | 0.28 |
| sihSIRPα_10 | 0.21 | 0.94 |
| sihSIRPα_11 | 0.05 | 0.13 |
| sihSIRPα_12 | 0.05 | 0.14 |
| sihSIRPα_13 | 0.05 | 0.15 |
| sihSIRPα_14 | 0.43 | 0.75 |
| sihSIRPα_15 | 0.10 | 0.34 |
| sihSIRPα_16 | 0.04 | 0.13 |
| sihSIRPα_17 | 0.05 | 0.17 |
| sihSIRPα_18 | 0.01 | 0.08 |
| sihSIRPα_19 | 0.03 | 0.15 |
| siLuc | 1.00 | 1.00 |

TABLE 11
Relative fold change of SIRPα mRNA level

| siRNA duplex ID | 25 nM | 5 nM |
|---|---|---|
| sihSIRPα_1-PM | 0.07 | 0.60 |
| sihSIRPα_2-PM | 0.17 | 0.60 |
| sihSIRPα_3-PM | 0.02 | 0.56 |
| sihSIRPα_4-PM | 0.05 | 0.60 |
| sihSIRPα_7-PM | 0.13 | 0.74 |
| sihSIRPα_8-PM | 0.06 | 0.77 |
| sihSIRPα_12-PM | 0.07 | 0.68 |
| sihSIRPα_13-PM | 0.07 | 0.66 |
| sihSIRPα_17-PM | 0.21 | 0.71 |
| sihSIRPα_18-PM | 0.01 | 0.72 |
| sihSIRPα_19-PM | 0.05 | 0.75 |
| siLuc | 1.00 | 1.00 |

### Example 3: Dose Response of Select Human SIRPα siRNA Duplexes and Variants

Human SIRPα siRNA duplexes and variants that resulted in a significant reduction of SIRPα mRNA level in the dual dose screening were selected and further tested for dose responses. In addition to these duplexes, 10 siRNA duplexes were also tested. THP-1 monocytes were cultured using the standard culture condition and transferred to 96-well plates at a density of 10,000-15,000 cells per well. THP-1 monocytes were differentiated into macrophages by an incubation with 100 ng/mL phorbol 12-myristate 13-acetate (PMA) for 24 hours and subsequent recovery period for 24 hours. THP-1 macrophages were transfected with SIRPα siRNA duplexes using Viromer Blue (0.5 μl/well). The doses for each SIRPα siRNA duplex included 25 nM, 2.5 nM, 250 pM, 25 pM. Following incubation of 48 hours, the treated cells were harvested and the remaining SIRPα mRNA level was measured in each condition by RT-qPCR. The IC₅₀ value of each SIRPα siRNA duplex was determined and each dose response is shown in Table 12 and Table 13.

TABLE 12
IC50 values of SIRPα siRNA duplexes

| | SIRPα mRNA relative fold change | | | | |
|---|---|---|---|---|---|
| siRNA duplex ID | 25 nM | 2.5 nM | 250 pM | 25 pM | IC₅₀ (pM) |
| sihSIRPα_1-PM | 0.06 | 0.33 | 0.72 | 0.81 | 984 |
| sihSIRPα_3-PM | 0.02 | 0.11 | 0.25 | 0.58 | 45 |
| sihSIRPα_4-PM | 0.05 | 0.14 | 0.54 | 0.85 | 291 |
| sihSIRPα_8-PM | 0.04 | 0.17 | 0.49 | 0.87 | 258 |
| sihSIRPα_12-PM | 0.10 | 0.30 | 0.65 | 0.99 | 712 |
| sihSIRPα_13-PM | 0.07 | 0.14 | 0.36 | 0.72 | 113 |
| sihSIRPα_16-PM | 0.06 | 0.11 | 0.29 | 0.71 | 83 |
| sihSIRPα_18-PM | 0.02 | 0.05 | 0.15 | 0.41 | 19 |
| sihSIRPα_19-PM | 0.06 | 0.16 | 0.41 | 0.69 | 131 |
| siLuc | 1.000 | 1.000 | 1.000 | 1.000 | |

TABLE 13
IC50 values of SIRPα siRNA duplexes

| | SIRPα mRNA relative fold change | | | | |
|---|---|---|---|---|---|
| siRNA duplex ID | 25 nM | 2.5 nM | 250 pM | 25 pM | IC₅₀ (pM) |
| sihSIRPα_20-PM | 0.12 | 0.23 | 0.46 | 0.77 | 221 |
| sihSIRPα_21-PM | 0.08 | 0.16 | 0.50 | 0.89 | 269 |
| sihSIRPα_22-PM | 0.03 | 0.09 | 0.26 | 0.52 | 36 |
| sihSIRPα_23-PM | 0.14 | 0.27 | 0.53 | 0.82 | 342 |
| sihSIRPα_24-PM | 0.12 | 0.19 | 0.44 | 0.74 | 180 |
| sihSIRPα_25-PM | 0.33 | 0.56 | 0.62 | 0.75 | 3220 |
| sihSIRPα_26-PM | 0.12 | 0.32 | 0.64 | 0.78 | 644 |
| sihSIRPα_27-PM | 0.02 | 0.10 | 0.33 | 0.76 | 114 |
| sihSIRPα_28-PM | 0.91 | 1.03 | 1.00 | 0.80 | 334800 |
| sihSIRPα_29-PM | 0.08 | 0.12 | 0.33 | 0.68 | 88 |
| siLuc | 1.000 | 1.000 | 1.000 | 1.000 | |

### Example 4: Dual Response Screening of Mouse SIRPα siRNA Duplexes and Variants

siRNA modification variants derived from siRNA duplexes with high efficiency and specificity to murine SIRPα RNA knock-down were designed according to sequence and chemical modifications (as described in Example 1) and the resulting duplex variants were further tested in J774 murine macrophage cell line. J774 cells were cultured using the standard culture condition and transferred to 96-well plates at a density of 10,000-15,000 cells per well. J774 macrophages were transfected with SIRPα siRNA duplexes using Viromer Blue (0.5 μl/well). A total of 36 siRNA duplexes, including variants from the original modified siRNA duplexes, were transfected into J774 cells and further validated. The SIRPα siRNAs were transfected at a final concentration of 5 nM and 25 nM, respectively. An anti fLuc siRNA duplex was used as a negative control. After incubating for 48 hours, total mRNA was extracted and purified, and cDNA was synthesized by reverse transcription. SIRPα expression level was quantified by real time PCR. The information and sequences of these siRNA duplexes are included in Table 14 and Table 15.

TABLE 14
Relative fold change of SIRPα mRNA level

| siRNA duplex ID | 25 nM | 5 nM |
|---|---|---|
| simSIRPα_1 | 0.53 | 0.77 |
| simSIRPα_2 | 0.84 | 0.85 |
| simSIRPα_3 | 0.21 | 0.33 |
| simSIRPα_4 | 0.36 | 0.68 |
| simSIRPα_5 | 0.18 | 0.39 |
| simSIRPα_6 | 0.30 | 0.60 |
| simSIRPα_7 | 0.15 | 0.50 |
| simSIRPα_8 | 0.21 | 0.48 |
| simSIRPα_9 | 1.40 | 0.81 |
| simSIRPα_10 | 0.19 | 0.43 |
| simSIRPα_11 | 0.18 | 0.67 |
| simSIRPα_12 | 0.13 | 0.49 |
| simSIRPα_13 | 0.09 | 0.55 |
| simSIRPα_14 | 0.23 | 0.70 |
| simSIRPα_15 | 0.13 | 0.44 |
| simSIRPα_16 | 0.26 | 0.63 |
| siLuc | 1.00 | 1.00 |

TABLE 15
Relative fold change of SIRPα mRNA level

| siRNA duplex ID | 25 nM | 2.5 nM |
|---|---|---|
| simSIRPα_3-PM | 0.03 | 0.06 |
| simSIRPα_5-PM | 0.05 | 0.21 |
| simSIRPα_10-PM | 0.15 | 0.19 |
| simSIRPα_11-PM | 0.15 | 0.09 |
| simSIRPα_13-PM | 0.24 | 0.13 |
| simSIRPα_17-PM | 0.80 | 1.01 |
| simSIRPα_18-PM | 0.09 | 0.31 |
| simSIRPα_19-PM | 0.05 | 0.12 |
| simSIRPα_20-PM | 0.10 | 0.25 |
| simSIRPα_21-PM | 0.06 | 0.07 |
| simSIRPα_22-PM | 0.08 | 0.13 |
| simSIRPα_23-PM | 0.04 | 0.21 |
| simSIRPα_24-PM | 0.55 | 0.78 |
| simSIRPα_25-PM | 0.04 | 0.10 |
| simSIRPα_26-PM | 0.37 | 0.74 |
| simSIRPα_27-PM | 0.53 | 0.83 |
| simSIRPα_28-PM | 0.23 | 0.45 |
| simSIRPα_29-PM | 0.09 | 0.12 |
| simSIRPα_30-PM | 0.07 | 0.21 |
| simSIRPα_31-PM | 0.03 | 0.05 |
| siLuc | 1.00 | 1.00 |

### Example 5: Dose Response of Selected Mouse SIRPα siRNA Duplexes and Variants

Mouse SIRPα siRNA duplexes and variants that resulted in a significant reduction of SIRPα mRNA level in the dual dose screening were selected and further tested for dose responses. J774 murine macrophage cell line was cultured using the standard culture condition and transferred to 96-well plates at a density of 10,000-15,000 cells per well. J774 cells were transfected with SIRPα siRNA duplexes using Viromer Blue (0.5 μl/well). The doses for each SIRPα siRNA duplex included 25 nM, 2.5 nM, 250 pM, 25 pM. Following incubation of 48 hours, the treated cells were harvested and the remaining SIRPα mRNA level was measured in each condition by RT-qPCR. The IC₅₀ value of each SIRPα siRNA duplex was determined and each dose response is shown in Table 16.

TABLE 16
IC50 values of SIRPα siRNA duplexes

| | SIRPα mRNA relative fold change | | | | |
|---|---|---|---|---|---|
| siRNA duplex ID | 25 nM | 2.5 nM | 250 pM | 25 pM | IC₅₀ (pM) |
| simSIRPα_3-PM | 0.05 | 0.16 | 0.51 | 0.76 | 232 |
| simSIRPα_19-PM | 0.19 | 0.23 | 0.59 | 0.80 | 437 |
| simSIRPα_21-PM | 0.07 | 0.12 | 0.39 | 0.68 | 123 |
| simSIRPα_25-PM | 0.08 | 0.24 | 0.43 | 0.76 | 221 |
| simSIRPα_31-PM | 0.05 | 0.10 | 0.41 | 0.73 | 147 |
| siLuc | 1.000 | 1.000 | 1.000 | 1.000 | |

### Example 6: Formulating siRNAs with Lipid Nanoparticles (LNPs)

LNPs were synthesized at a composition of 50:10:38.5:1.5 molar ratio of ionizable lipid:1,2-distearoyl-sn-glycero-3-phosphocholine (DSPC):cholesterol:1,2-dimyristoyl-rac-glycero-3-methoxypolyethylene glycol-2000 (DMG-PEG2000) and an ionizable lipid:siRNA weight ratio of 10:1.

Particle size and PDI values were obtained by a Zeta Sizer (Malvern) and siRNA concentration and encapsulation efficiency were evaluated by modified Ribo-Green assay (Thermo Fisher).

TABLE 17
Composition of LNP-formulated SIRPα siRNAs

| | | | Molar ratio | | | | ionizable |
|---|---|---|---|---|---|---|---|
| | | Ionizable | Ionizable | | | DMG- | lipid/siRNA |
| | siRNA | lipid | lipid | DSPC | Cholesterol | PEG2000 | weight ratio |
| LNP1 | sihSIRPα_3-PM | FL-A | 50 | 10 | 38.5 | 1.5 | 10 |
| LNP2 | sihSIRPα_18-PM | FL-A | 50 | 10 | 38.5 | 1.5 | 10 |
| LNP3 | sihSIRPα_22-PM | FL-A | 50 | 10 | 38.5 | 1.5 | 10 |
| LNP4 | siLuc | FL-A | 50 | 10 | 38.5 | 1.5 | 10 |
| LNP5 | siCD45 | FL-A | 50 | 10 | 38.5 | 1.5 | 10 |
| LNP6 | simSIRPα_21-PM | FL-A | 50 | 10 | 38.5 | 1.5 | 10 |
| LNP7 | sihSIRPα_18-PM | MC3 | 50 | 10 | 38.5 | 1.5 | 10 |
| LNP8 | sihSIRPα_18-PM | Lipid 5 | 50 | 10 | 38.5 | 1.5 | 10 |

TABLE 18
Physiochemical properties of LNPs

| | Particle size | | Encapsulation |
|---|---|---|---|
| | (nm) | PDI | efficiency (%) |
| LNP1 | 125.3 | 0.05 | 94 |
| LNP2 | 137.1 | 0.10 | 92 |
| LNP3 | 136.4 | 0.05 | 93 |
| LNP4 | 92.6 | 0.05 | 97 |
| LNP5 | 107.2 | 0.22 | 95 |
| LNP6 | 116.7 | 0.07 | 93 |
| LNP7 | 95.1 | 1.2 | 92 |
| LNP8 | 100.1 | 0.8 | 90 |

### Example 7: LNP Enables Myeloid Cell-Specific RNA Delivery In Vivo

To examine which immune cell type the LNPs can deliver siRNA, LNP-containing siRNAs against CD45 (siCD45) were prepared. CD45, a cell surface tyrosine phosphatase, was chosen since it is ubiquitously expressed on all the immune cell types and thus can be used for testing gene silencing in different immune cell subsets. Mice were intraperitoneally injected with siCD45-LNPs or PBS.

siCD45 siRNA Sequence

(SEQ ID NO: 228)
sense: mCmUGGmCmUGAAmUmUmUmCAGAGmCA-dTdT,
(SEQ ID NO: 229)
antisense: UGCUCUGAAAUUmCAGCmCAG-dTdT

Three days post-administration, peritoneal lavage was performed to harvest immune cells in peritoneal cavity, and these immune cells were stained for markers of different immune cell subsets, and analyzed by flow cytometry.

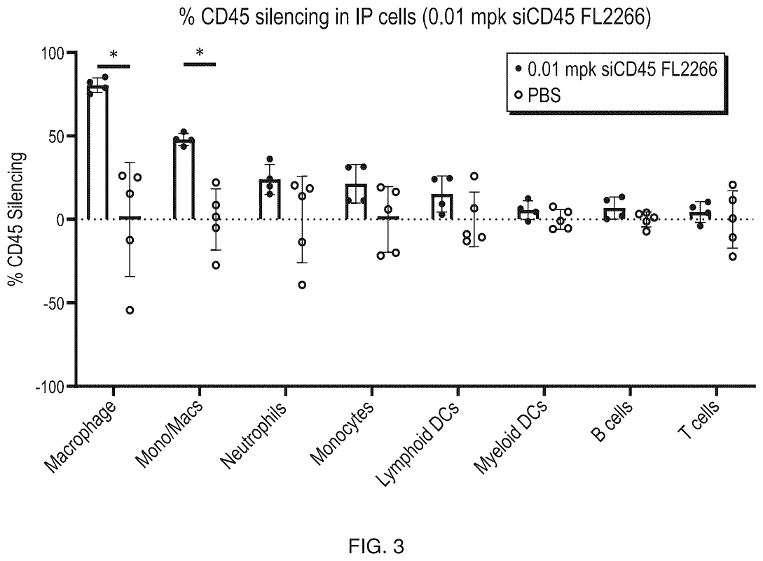

Remarkably, macrophages and their precursors (Mono/Macs) showed statistically significant CD45 gene silencing at 0.01 mg/kg, whereas lymphocytes such as B cells and T cells didn't show significant gene silencing (FIG. 3).

This macrophage-tropic nature of the LNPs should be useful to further suppress off-target gene silencing of SIRPγ in T cells.

### Example 8: siSIRPα-LNP Shows Gene Silencing in Mice

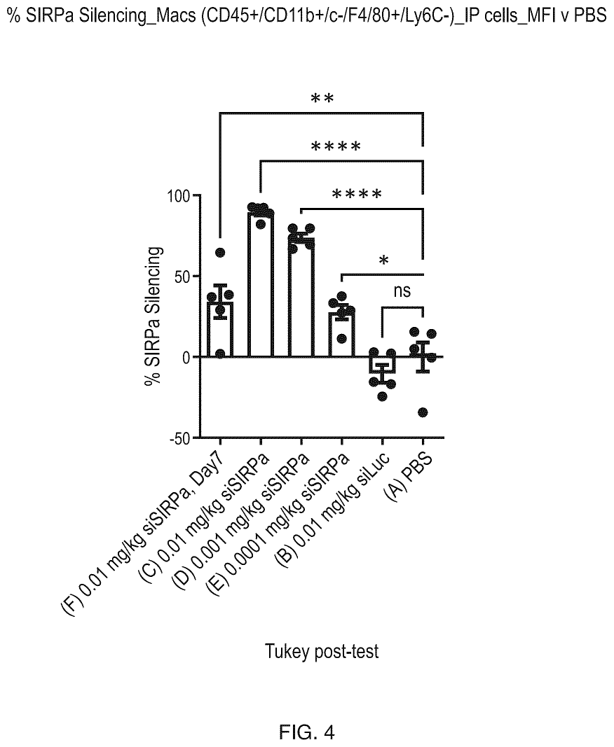

To optimize the dosing amount and schedule of siSIRPα-LNP in vivo, mice were intraperitoneally injected with siSIRPα-LNPs, siLuc-LNP, or PBS. Three days or seven days post-administration, gene silencing in peritoneal immune cells was analyzed by flow cytometry. The siSIRPα-LNP showed a dose-dependent SIRPα gene silencing and its effective dose 50 (ED50) was 0.00025 mg/kg (FIG. 4)

### Example 9: siSIRPα-LNP Promotes M1 Phenotype in Primary Human Macrophages

To investigate whether SIRPα gene silencing on macrophages can reprogram tumor-associated macrophage phenotype from M2 to M1, the expression level of macrophage polarization markers and antigen presenting molecules was evaluated.

Human primary monocytes were isolated from peripheral blood mononuclear cells by Pan Monocyte Isolation Kit, human (Miltenyi Biotec). These monocytes were differentiated into macrophage with M-CSF (20 ng/ml for 4 days, followed by 40 ng/ml for 4 days) and then transfected with FL-A LNP-formulated siSIRPα or siLuc (50 nM siRNA dose) on day 8. These cells were harvested and analyzed by flow cytometry on day 3 after transfection. 20 ng/mL M-CSF was maintained in the culture media following transfection.

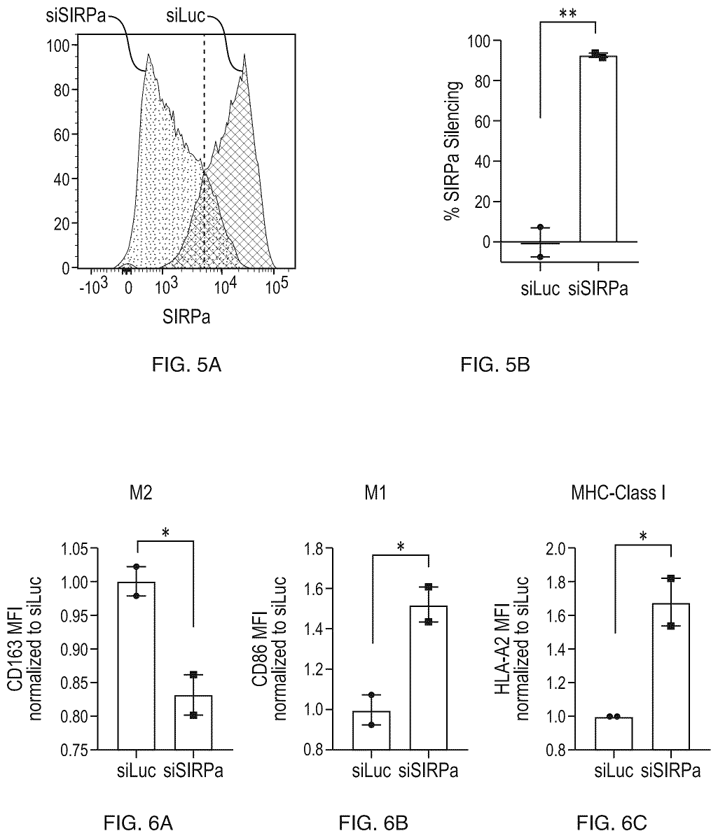

SIRPα siRNA showed about 90% SIRPα gene silencing on human primary macrophages (FIGS. 5A, 5B). In addition, SIRPα silencing reduced M2 maker expression (CD163) and increased M1 marker (CD86). These results strongly suggest that SIRPα siRNA could reprogram tumor microenvironment (FIGS. 6A-6C). Also, upregulation of antigen presenting molecules (MHC-I) suggest that SIRPα siRNA could enhance antigen presentation on macrophages and other myeloid cells (FIGS. 6A-6C).

### Example 10: siSIRPα Sequences and LNP Formulations are Generalizable (1)

To investigate whether other siRNA sequences and LNP formulations can silence SIRPα expression and reprogram tumor-associated macrophage phenotype from M2 to M1, the expression level of macrophage polarization markers and antigen presenting molecules with various siRNA sequences and LNP formulations was evaluated.

Human primary monocytes were isolated from peripheral blood mononuclear cells by Pan Monocyte Isolation Kit, human (Miltenyi Biotec). These monocytes were differentiated into macrophage with M-CSF (20 ng/ml for 4 days, followed by 40 ng/ml for 4 days) and then transfected with LNP-formulated siSIRPα or siLuc (50 nM siRNA dose) on day 8. Lipofectamine® RNAiMax (RIM)-formulated siSIRPα and siLuc were also tested. These cells were harvested and analyzed by flow cytometry on day 3 after transfection. 20 ng/mL M-CSF was maintained in the culture media following transfection.

The experimental protocol is further described below in Example 12.

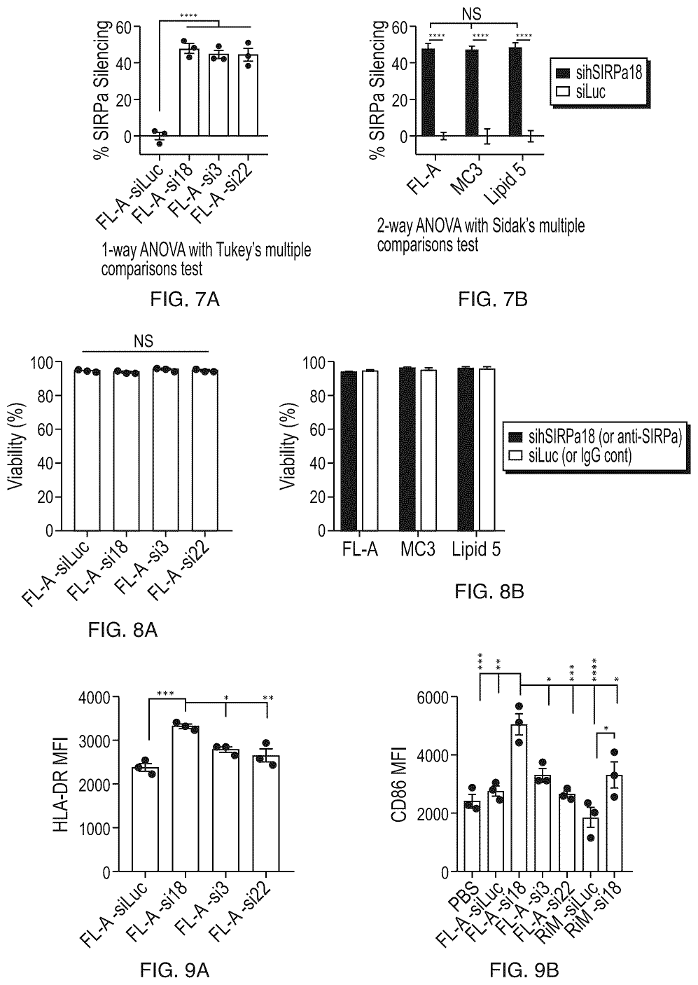

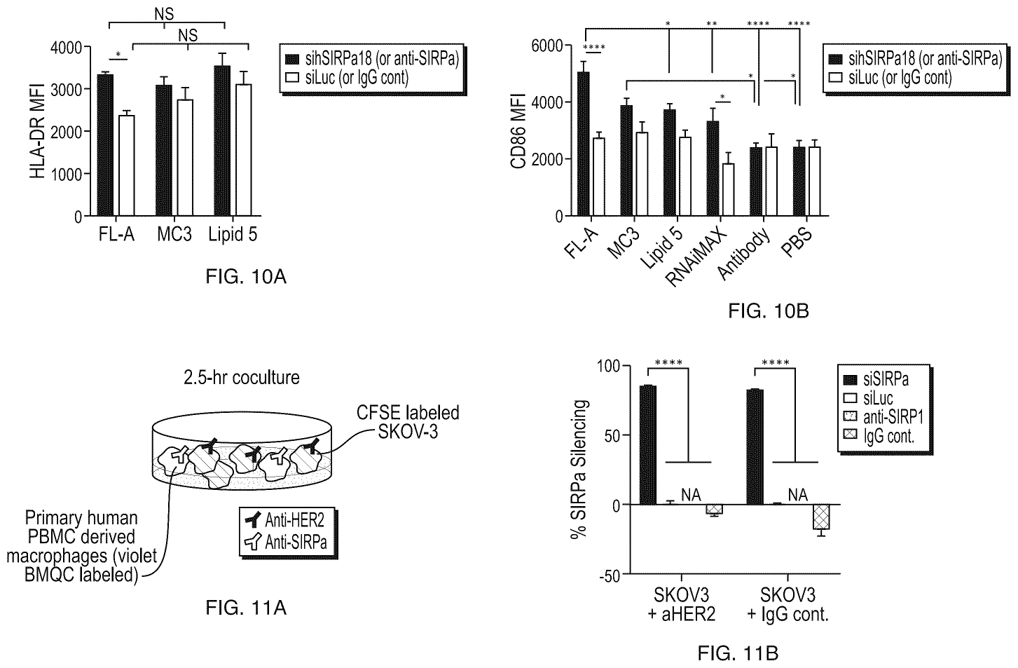

All the SIRPα siRNA sequences formulated with FL-A and all the LNPs formulated with sihSIRPα_18-PM (si18) showed about 50% SIRPα gene silencing on human primary macrophages, while maintaining almost 100% cell viability (FIGS. 7A, 7B; FIGS. 8A, 8B). In addition, among all the siRNAs tested, si18 showed most significant upregulation of M1 marker (CD86) and MHC-II antigen presenting molecules (HLA-DR). suggesting that si18 has higher immunogenicity than the other siRNAs (FIGS. 9A, 9B). With respect to ionizable lipids, FL-A induced most significant CD86 and HLA-DR upregulation, indicating that FL-A is most suitable for macrophage delivery for cancer application (FIGS. 10A, 10B). Based on these results, sihSIRPα_18-PM formulated with FL-A was selected for more detailed evaluation (simply referred to as siSIRPα-LNP hereafter).

TABLE 19
The list of siRNA-LNP tested in human primary macrophages

| | | | Molar ratio | | | | ionizable |
|---|---|---|---|---|---|---|---|
| | | Ionizable | Ionizable | | | DMG- | lipid/siRNA |
| | siRNA | lipid | lipid | DSPC | Cholesterol | PEG2000 | weight ratio |
| LNP1 | sihSIRPα_3-PM | FL-A | 50 | 10 | 38.5 | 1.5 | 10 |
| LNP2 | sihSIRPα_18-PM | FL-A | 50 | 10 | 38.5 | 1.5 | 10 |
| LNP3 | sihSIRPα_22-PM | FL-A | 50 | 10 | 38.5 | 1.5 | 10 |
| LNP7 | sihSIRPα_18-PM | MC3 | 50 | 10 | 38.5 | 1.5 | 10 |
| LNP8 | sihSIRPα_18-PM | Lipid 5 | 50 | 10 | 38.5 | 1.5 | 10 |
| LNP4 | siLuc | FL-A | 50 | 10 | 38.5 | 1.5 | 10 |

### Example 11: siSIRPα Sequences and LNP Formulations are Generalizable (2)

To investigate whether other siRNA sequences and LNP formulations can silence SIRPα expression, the SIRPα expression level was evaluated with various siRNA sequences and LNP formulations using THP-1 derived macrophages. The following ionizable lipids were tested, and LNP formulations were listed in Table 20.

FL-A (2-butyloctyl 3-ethyl-12-hexyl-6-(2-(octanoyloxy)ethyl)-10-oxo-9,11-dioxa-3,6-diazahenicosan-21-oate) described in WO 2019/235635 A1:

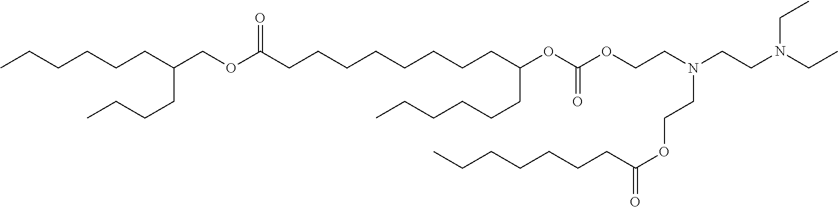 [SMILES: CCCCCCCC(=O)OCCN(CCOC(=O)OC(CCCCCC)CCCCCCCCC(=O)OCC(CCCC)CCCCCC)CCN(CC)CC]

GCL1; ([(6Z,16Z)-12-[(Z)-dec-4-enyl]docosa-6,16-dien-11-yl]5-(dimethylamino)pentanoate) described in WO 2020/219941 A1:

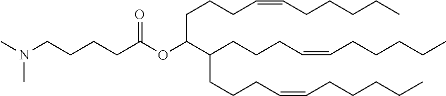 [SMILES: CCCCC/C=C\CCCC(CCC/C=C\CCCCC)C(CCC/C=C\CCCCC)OC(=O)CCCCN(C)C]

Lipid A9; (bis(2-butyloctyl) 10-[3-(dimethylamino)propyl-nonanoylamino]nonadecanedioate) described in WO 2017/004143 A1:

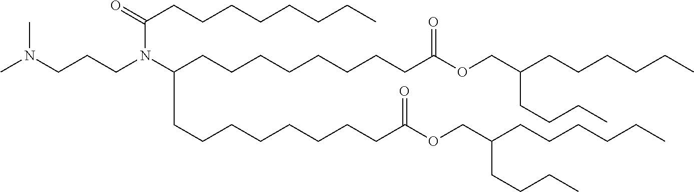 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

ALC-0315; (6-[6-(2-hexyldecanoyloxy)hexyl-(4-hydroxybutyl)amino]hexyl 2-hexyldecanoate) described in WO 2017/075331 A1:

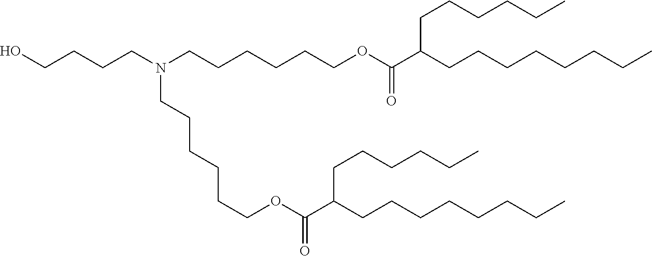 [SMILES: CCCCCCCCC(CCCCCC)C(=O)OCCCCCCN(CCCCO)CCCCCCOC(=O)C(CCCCCC)CCCCCCCC]

Lipid 5; (nonyl 8-[(8-heptadecan-9-yloxy-8-oxooctyl)-(2-hydroxyethyl)amino]octanoate) described in WO 2017/099823 A1:

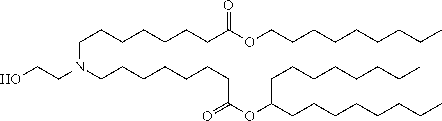 [SMILES: CCCCCCCCCOC(=O)CCCCCCCN(CCO)CCCCCCCC(=O)OC(CCCCCCCC)CCCCCCCC]

TABLE 20
List of siRNA-LNP tested in THP-1-derived macrophages

| | | | Molar ratio | | | | ionizable |
|---|---|---|---|---|---|---|---|
| | siRNA | Ionizable | Ionizable | | | DMG- | lipid/siRNA |
| | duplex ID | lipid | lipid | DSPC | Cholesterol | PEG2000 | weight ratio |
| LNP1 | sihSIRPα_3-PM | FL-A | 50 | 10 | 38.5 | 1.5 | 10 |
| LNP2 | sihSIRPα_18-PM | FL-A | 50 | 10 | 38.5 | 1.5 | 10 |
| LNP3 | sihSIRPα_22-PM | FL-A | 50 | 10 | 38.5 | 1.5 | 10 |
| LNP9 | sihSIRPα_3-PM | A9 | 50 | 10 | 38.5 | 1.5 | 10 |
| LNP10 | sihSIRPα_18-PM | A9 | 50 | 10 | 38.5 | 1.5 | 10 |
| LNP11 | sihSIRPα_22-PM | A9 | 50 | 10 | 38.5 | 1.5 | 10 |
| LNP12 | sihSIRPα_3-PM | Lipid 5 | 50 | 10 | 38.5 | 1.5 | 10 |
| LNP8 | sihSIRPα_18-PM | Lipid 5 | 50 | 10 | 38.5 | 1.5 | 10 |
| LNP13 | sihSIRPα_22-PM | Lipid 5 | 50 | 10 | 38.5 | 1.5 | 10 |
| LNP14 | sihSIRPα_3-PM | GCL1 | 50 | 10 | 38.5 | 1.5 | 10 |
| LNP15 | sihSIRPα_18-PM | GCL1 | 50 | 10 | 38.5 | 1.5 | 10 |
| LNP16 | sihSIRPα_22-PM | GCL1 | 50 | 10 | 38.5 | 1.5 | 10 |
| LNP17 | sihSIRPα_3-PM | ALC-0315 | 50 | 10 | 38.5 | 1.5 | 10 |
| LNP18 | sihSIRPα_18-PM | ALC-0315 | 50 | 10 | 38.5 | 1.5 | 10 |
| LNP19 | sihSIRPα_22-PM | ALC-0315 | 50 | 10 | 38.5 | 1.5 | 10 |

The experimental protocol was as written in Example 3. The doses for each SIRPα˜siRNA duplex included 50 nM and 5 nM.

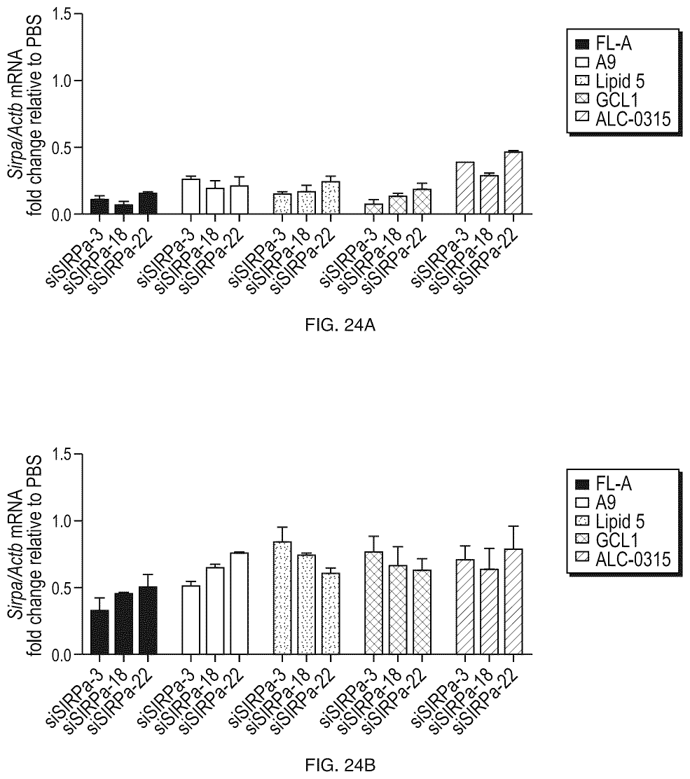

All the SIRPα siRNA sequences and LNP formulations showed more than 50% SIRPα gene silencing on THP-1 derived macrophages at 50 nM (FIGS. 24A, 24B). Based on these results, it was concluded that siRNA sequences selected in the screening are potent enough to show gene silencing when formulated with various ionizable lipids.

### Example 11: Comparison Between siRNA and Antibody Against SIRPα

### Ovarian Cancer Phagocytosis

In order to compare SIPRa gene silencing driven by siSIRPα to antibody blocking of SIRPα activity, primary human macrophages were pretreated with siSIRPα-LNPs (or siLuc-LNP as a negative control) or anti-SIRPα (or the isotype (IgG) control antibody) prior to coculture with human ovarian cancer cells (SKOV-3). Prior to initiating the coculture, these pretreated macrophages were also labeled with a violet BMQC cell tracker fluorescent dye (Invitrogen by ThermoFisher Scientific #C10094). SKOV-3 ovarian cancer cells were stained with the green fluorescent CFSE cell tracker (BioLegend). In some conditions, SKOV-3 cells were pretreated with anti-HER2 antibodies (as in Example 12). Macrophages and SKOV-3 ovarian cancer cells were seeded at a ratio of 1 to 2, and incubated as a coculture for 2.5 hours (FIGS. 11A, 11B). Flow cytometry was used to quantify SIRPα silencing, expression level of macrophage polarization markers and antigen presentation molecules, and phagocytosis.

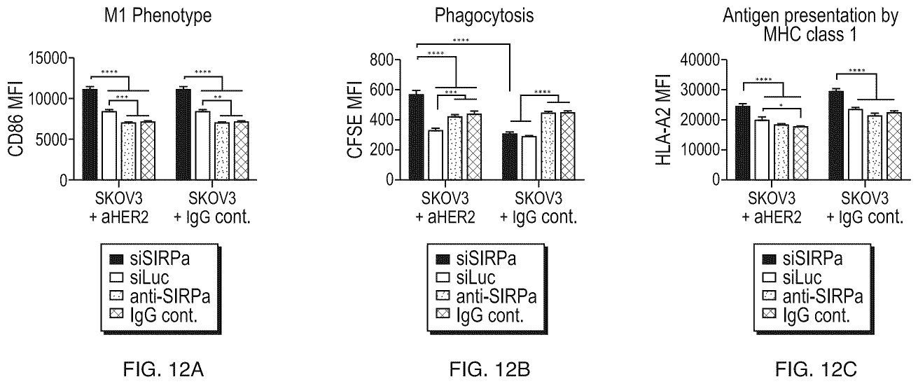

siSIRPα treatment resulted in >80% SIRPα gene silencing on primary human macrophages (FIGS. 11A, 11B). SIRPα gene silencing with siSIRPα also increased M1 marker expression, as compared to SIRPα blocking with a blocking antibody (FIGS. 12A-12C). The intensity of CFSE fluorescence within the macrophage population indicates phagocytosis by the macrophages. Increased CFSE fluorescence within the CD45+ macrophage population following siSIRPα treatment indicates that siSIRPα treatment increased SKOV-3 ovarian cancer phagocytosis by the macrophages, as compared to blocking SIRPα with a blocking antibody (FIGS. 12A-12C). Further, the level of expression of the class 1 antigen presentation molecule (HLA-A2) within the CD45+ macrophage population was increased following siSIRPα treatment as compared to treatment with the SIRPα blocking antibody (FIGS. 12A-12C).

### Mutual Phagocytosis

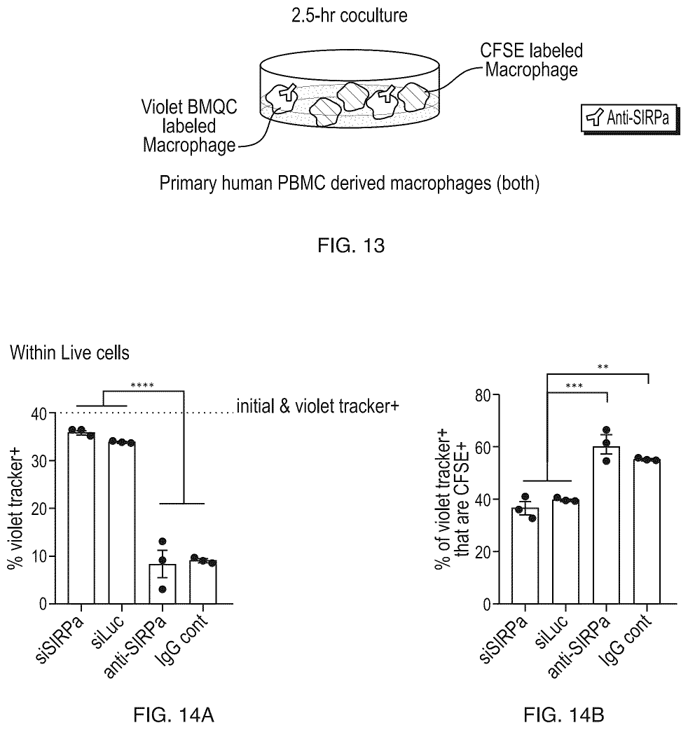

Mutual phagocytosis is the phagocytosis of macrophages by other macrophages. In order to compare mutual phagocytosis following treatment with either siSIRPα or anti-SIRPα blocking antibodies, primary human macrophages were treated with siSIRPα-LNP, siLuc-LNP (as a negative control), anti-SIRPα, or IgG control antibody (as a negative control). The treated macrophages were labeled with a violet BMQC cell tracker fluorescent dye (Invitrogen by ThermoFisher Scientific #C10094), and placed in coculture with untreated macrophages. The untreated macrophages were labeled with the green fluorescent CFSE cell tracker (BioLegend). Violet (i.e., pretreated) macrophages and green (i.e., untreated) macrophages were seeded at a ratio of 1 to 1.5, and incubated as a coculture for 2.5 hours (FIG. 13). Flow cytometry was then used to quantify the remaining violet, and green labeled macrophages, and assess mutual phagocytosis.

Macrophages treated with either anti-SIRPα blocking antibody, or the IgG control antibody, were substantially depleted following the 2.5-hour coculture, whereas macrophages treated with siSIRPα-LNP or siLuc-LNP were not (FIGS. 14A, 14B). This indicates that treatment with anti-SIRPα blocking antibodies renders macrophages more susceptible to mutual phagocytosis than treatment with siSIRPα. The mutual phagocytotic activity of the violet (i.e., pretreated) macrophages was quantified as the percentage of violet macrophages that are positive for the CFSE cell tracker. Macrophages that had been pretreated with the anti-SIRPα blocking antibody demonstrated more phagocytosis of other (i.e., green) macrophages than macrophage that had been pretreated with siSIRPα (FIGS. 14A, 14B).

### Example 13: siSIRPα-LNP+aHER2 Antibody Promotes Ovarian Cancer Cell Phagocytosis by Primary Human Macrophages

In order to determine if SIRPα gene silencing on macrophages is synergistic with antibody therapeutics, primary human macrophages that had been transfected with siRNA-LNP (siSIRPα or siLUC as a negative control) were cocultured with human ovarian cancer cells (SKOV-3 cells) that had been preincubated with an antibody therapeutic (aHER2).

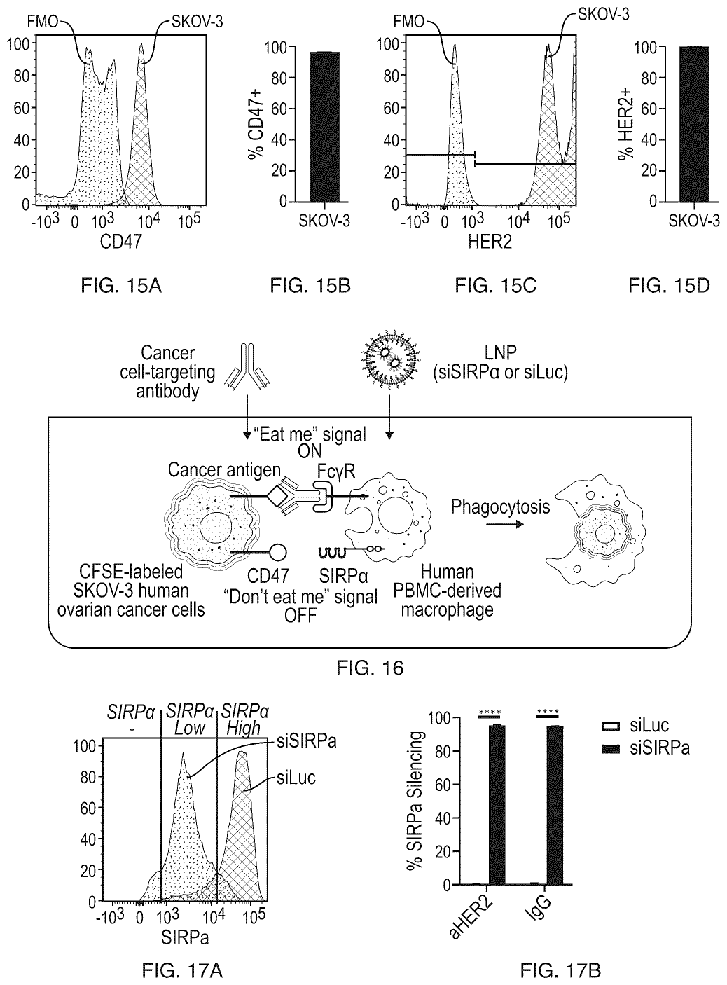

Here, SKOV-3 human ovarian cancer cells were observed to express high levels of the SIRPα ligand CD47 (FIGS. 15A, 15B), suggesting that CD47-SIRPα interactions may contribute to macrophage inhibition, and the ability of SKOV-3 ovarian cancer to evade host immune defenses. CD47 engagement with macrophage-expressed SIRPα triggers an inhibitory cascade that prevents macrophage phagocytosis of the target (SKOV-3) cell bearing CD47, i.e. a “don't eat me” OFF signal. Conversely, antibodies capable of engaging Fc receptors on macrophages trigger a stimulatory cascade leading to macrophage activation and phagocytosis, i.e. an “eat me” ON signal (FIG. 16). SKOV-3 ovarian cancer cells express HER2 (FIGS. 15C, 15D), suggesting that anti-HER2 antibodies may opsonize SKOV-3 cells, engage Fc receptors on macrophages, and provide a positive signal for phagocytosis (FIG. 16).

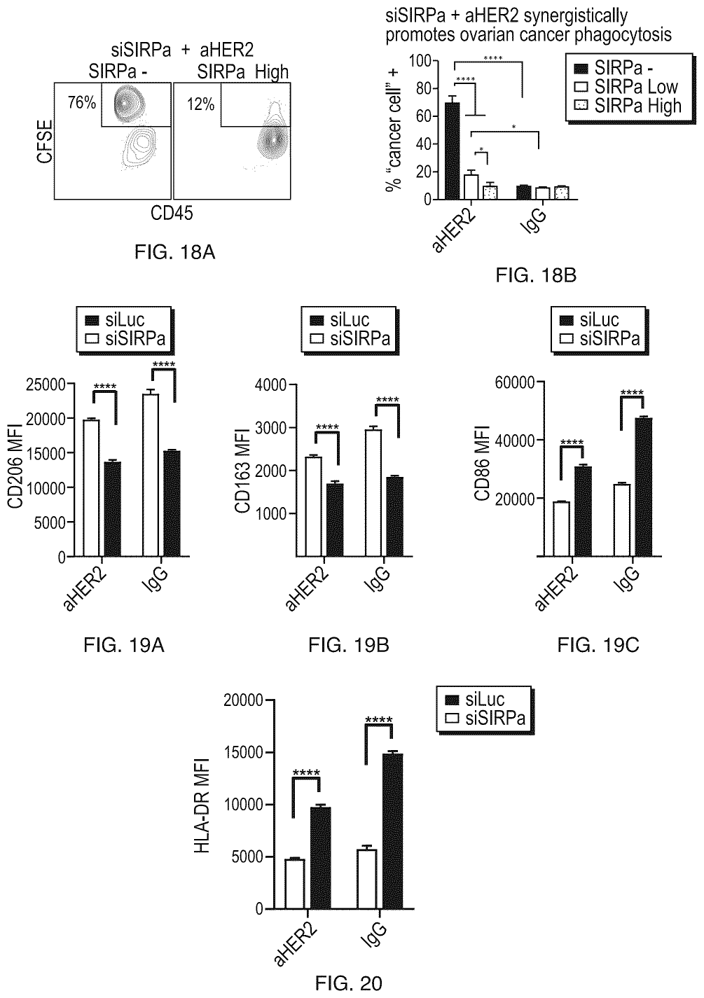

Primary human monocytes were isolated from peripheral blood mononuclear cells and differentiated into macrophages as described in Example 12. The primary human macrophages were transfected with siRNA-LNP as described in Example 12, and used in the cocultures (described below) 3 days after transfection. SKOV-3 ovarian cancer cells were labeled with a fluorescent cell tracker, CFSE (BioLegend), and pre-incubated with anti-HER2 or an IgG isotype control antibody prior to coculture. After a 2 hour coculture of the SKOV-3 cancer cells with the transfected macrophages, flow cytometry was used to quantify the level of SIRPα silencing (FIG. 17A, 17B), the percentage of macrophages positive for the CFSE cancer cell tracker (indicating phagocytosis) (FIG. 18A, 18B), the expression of M1 versus M2 macrophage phenotype markers (FIG. 19A-19C), and the expression of antigen presenting molecules (FIG. 20).

siSIRPα-LNP resulted in over 90% SIRPα gene silencing in primary human macrophages (FIG. 17A, 17B). SIRPα silencing in combination with anti-HER2 cancer treatment synergistically promoted macrophage phagocytosis of the SKOV-3 ovarian cancer cells (FIG. 18A, 18B). The level of phagocytosis observed following siSIRPα+aHER2 (about 70%) treatments exceed that observed following siSIRPα silencing or anti-HER2 treatment alone (FIG. 18A, 18B). In addition, SIRPα silencing reduced M2 marker expression (CD206, CD163 (FIG. 19A, 19B)) and increased M1 marker expression (CD86) (FIG. 19C). These results strongly suggest that SIRPα siRNA could reprogram the tumor microenvironment. Upregulation of the class II antigen presenting molecule (HLA-DR) (FIG. 20) suggests that SIRPα siRNA may enhance antigen presentation by primary human macrophages.

### Example 14: siSIRPα-LNP Promotes Phagocytosed Antigen Cross-Presentation In Vitro

Cross-presentation is the ability of certain professional antigen-presenting cells (primarily macrophages and dendritic cells) to internalize, process, and present extracellular antigens on MHC class I molecules. Because antigen cross-presentation has the ability to initiate antigen-specific CD8 T cell responses, stimulating macrophages to cross-present tumor antigens is of substantial interest.

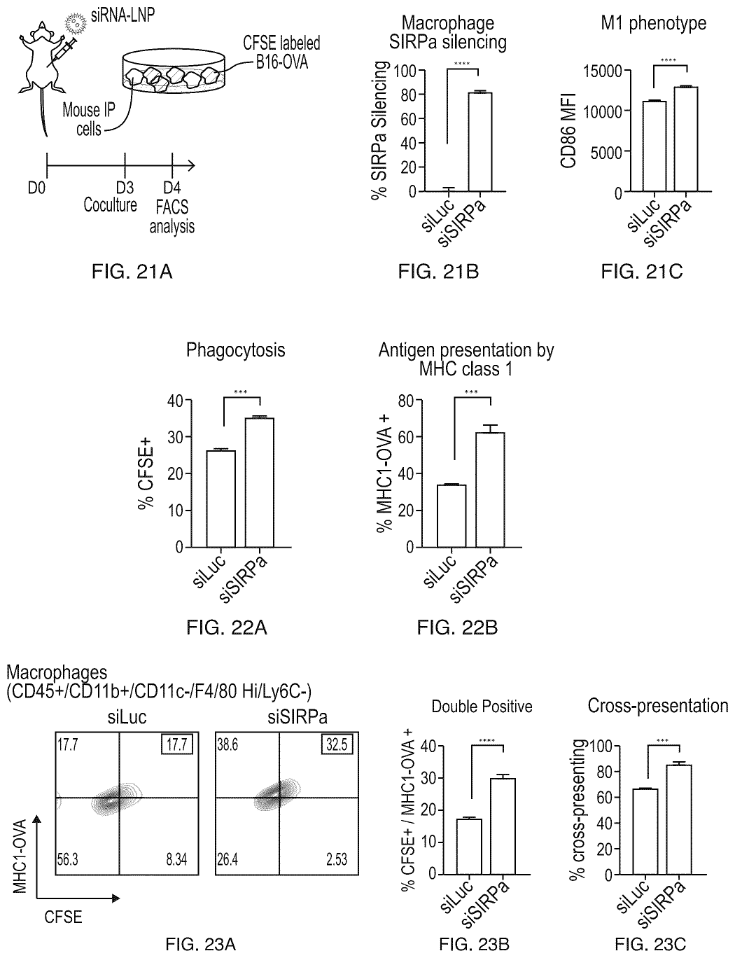

In order to determine if SIRPα gene silencing promotes cross-presentation of tumor antigens by macrophages, murine peritoneal cells which had been treated with an intraperitoneal injection of siSIRPα-LNP (or siLUC-LNP as a negative control) were harvested 3 days after siRNA treatment and placed in coculture with B16 murine melanoma cells (FIG. 21A-21C). The B16 melanoma cells expressed ovalbumin (OVA), which was used as a model cancer antigen. In order to track the B16 melanoma cells, and quantify phagocytosis, the B16 cells were stained with CSFE cell tracker before placing in coculture. Following an overnight coculture of the labeled B16 melanoma cells with the murine IP cells, flow cytometry was used to identify macrophages, and within the macrophage population: assess SIRPα silencing, quantify phagocytosis of B16 melanoma and cancer antigen presentation on MHC class I, and assess phenotype.

Intraperitoneal injection of siSIRPα-LNP resulted in significant silencing of SIRPα expression on murine peritoneal macrophages, and increased their expression of CD86 indicating a shift towards the M1 phenotype (FIGS. 21A-C). Macrophage phagocytosis of B16 antigen was quantified as the percentage of macrophages positive for the B16 cell tracker CFSE. siSIRPα-LNP treatment significantly increased the percentage of macrophages demonstrating B16 phagocytosis (FIGS. 22A, 22B). Importantly, macrophage presentation of the model antigen (OVA) on MHC class I was also significantly increased by siSIRPα-LNP treatment (FIGS. 22A, 22B). Cross-presentation requires the internalization of extracellular antigens and their subsequent presentation on MHC class I. Therefore, in order to more directly quantify cross-presentation, flow cytometry was used to simultaneously assess B16 antigen uptake (indicated by CFSE) and MHC class I OVA presentation on the macrophages (FIGS. 23A-C). The percentage of cross-presenting macrophages was quantified as the fraction of CFSE+ macrophages that were also MHC class I-OVA positive.

$$\% cross presentation = ( MHC - OVA + CFSE + ) / ( CFSE + ) ⋆ 100$$

The percentage of cross-presenting macrophages was significantly increased following intraperitoneal siSIRPα-LNP treatment (FIG. 23A-23C). Together, these results strongly suggest that SIRPα siRNA can promote macrophage activation (M1 phenotype) and cancer antigen cross-presentation by macrophages, to ultimately promote a strong anti-cancer immune response in cancer patients.

### REFERENCES

Barclay, A. Neil, and Deborah Hatherley. “The counterbalance theory for evolution and function of paired receptors.” Immunity 29.5 (2008): 675-678.
Dietrich, Jes, et al. “Cutting edge: signal-regulatory protein β1 is a DAP12-associated activating receptor expressed in myeloid cells.” The Journal of Immunology 164.1 (2000): 9-12.
Kurlander, R. J. “Blockade of Fc receptor-mediated binding to U-937 cells by murine monoclonal antibodies directed against a variety of surface antigens.” The Journal of Immunology 131.1 (1983): 140-147.
Madeira, Fibio, et al. “The EMBL-EBI search and sequence analysis tools APIs in 2019.” Nucleic acids research 47.W1 (2019): W636-W641.
Muntjewerff, Elke M., Luca D. Meesters, and Geert Van den Bogaart. “Antigen cross-presentation by macrophages.” Frontiers in Immunology 11 (2020): 1276.
Piccio, Laura, et al. “Adhesion of human T cells to antigen-presenting cells through SIRP02-CD47 interaction costimulates T-cell proliferation.” Blood 105.6 (2005): 2421-2427.
Stefanidakis, Michael, et al. “Endothelial CD47 interaction with SIRPγ is required for human T-cell transendothelial migration under shear flow conditions in vitro.” Blood, The Journal of the American Society of Hematology 112.4 (2008): 1280-1289.
US 2018/0312600
Voets, Erik, et al. “Functional characterization of the selective pan-allele anti-SIRPα antibody ADU-1805 that blocks the SIRPα-CD47 innate immune checkpoint.” Journal for immunotherapy of cancer 7.1 (2019): 1-15.
WO 2010/054405 A1
WO 2017/178653 A2
WO 2019/235635 A1
Ye, Xiaojing, et al. “Signal regulatory protein α associated with the progression of oral leukoplakia and oral squamous cell carcinoma regulates phenotype switch of macrophages.” Oncotarget 7.49 (2016): 81305.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1-56. (canceled)

57. A method of reducing signal regulatory protein alpha (SIRPα) expression in a cell, the method comprising contacting the cell with a nanoparticle composition comprising a nucleic acid signal regulatory protein alpha (SIRPα) therapeutic.

58. The method of claim 57, wherein the SIRPα therapeutic comprises a polynucleotide having at least 80% identity to a contiguous sequence within SEQ ID NO:4, SEQ ID:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, or SEQ ID NO:17, or a combination thereof, wherein the contiguous nucleotide sequence is at least about 15 nucleotides in length.

59. The method of claim 58, wherein the polynucleotide sequence further comprises at least one modified pyrimidine, and the at least one modified pyrimidine is 2′-O-methylcytidine-3′-phosphate or 2′-O-methyluridine-3′-phosphate.

60. The method of claim 59, wherein the SIRPα therapeutic is an siRNA duplex.

61. The method of claim 60, wherein the siRNA duplex comprises SEQ ID NO: 140 and SEQ ID NO: 141; SEQ ID NO: 142 and SEQ ID NO: 143; SEQ ID NO: 144 and SEQ ID NO: 145; SEQ ID NO: 146 and SEQ ID NO: 147; SEQ ID NO: 148 and SEQ ID NO: 149; SEQ ID NO: 150 and SEQ ID NO: 151; SEQ ID NO: 152 and SEQ ID NO: 153; SEQ ID NO: 154 and SEQ ID NO: 155; SEQ ID NO: 156 and SEQ ID NO: 157; SEQ ID NO: 158 and SEQ ID NO: 159; SEQ ID NO: 160 and SEQ ID NO: 161; SEQ ID NO: 162 and SEQ ID NO: 163; SEQ ID NO: 164 and SEQ ID NO: 165; SEQ ID NO: 166 and SEQ ID NO: 167; SEQ ID NO: 168 and SEQ ID NO: 169; SEQ ID NO: 170 and SEQ ID NO: 171; SEQ ID NO: 172 and SEQ ID NO: 173; SEQ ID NO: 174 and SEQ ID NO: 175; SEQ ID NO: 176 and SEQ ID NO: 177; SEQ ID NO: 178 and SEQ ID NO: 179; SEQ ID NO: 180 and SEQ ID NO: 181; SEQ ID NO: 182 and SEQ ID NO: 183; SEQ ID NO: 184 and SEQ ID NO: 185; SEQ ID NO: 186 and SEQ ID NO: 187; SEQ ID NO: 188 and SEQ ID NO: 189; SEQ ID NO: 190 and SEQ ID NO: 191; SEQ ID NO: 192 and SEQ ID NO: 193; SEQ ID NO: 194 and SEQ ID NO: 195; SEQ ID NO: 196 and SEQ ID NO: 197; SEQ ID NO: 198 and SEQ ID NO: 199; SEQ ID NO: 200 and SEQ ID NO: 201; SEQ ID NO: 202 and SEQ ID NO: 203; SEQ ID NO: 204 and SEQ ID NO: 205; SEQ ID NO: 206 and SEQ ID NO: 207; SEQ ID NO: 208 and SEQ ID NO: 209; SEQ ID NO: 210 and SEQ ID NO: 211; SEQ ID NO: 212 and SEQ ID NO: 213; SEQ ID NO: 214 and SEQ ID NO: 215; SEQ ID NO: 216 and SEQ ID NO: 217; SEQ ID NO: 218 and SEQ ID NO: 219; SEQ ID NO: 220 and SEQ ID NO: 221; SEQ ID NO: 222 and SEQ ID NO: 223; SEQ ID NO: 224 and SEQ ID NO: 225; or SEQ ID NO: 226 and SEQ ID NO: 227.

62. The method of claim 61, wherein the lipid nanoparticle comprises an ionizable lipid, phospholipid, sterol, polymer-conjugated lipid, or any combination thereof.

63. The method of claim 62, wherein the ionizable lipid is selected from the group consisting of FL-A; MC3; Lipid 5; Lipid A9; ALC-0315; and GCL1.

64. A composition comprising a nanocarrier selected from the group consisting of a lipid, a polymer, and a lipo-polymer hybrid, wherein the nanocarrier encapsulates a nucleic acid signal regulatory protein alpha (SIRPα) therapeutic; wherein SIRPα concentration in a cell contacted with the composition is reduced compared to SIRPα concentration in an otherwise identical cell.

65. A method of treating a signal regulatory protein alpha (SIRPα)-mediated disease or condition, comprising administering to subject in need thereof a lipid nanoparticle (LNP) composition comprising a nucleic acid signal regulatory protein alpha (SIRPα) therapeutic.

66. A method of treating cancer, comprising administering to subject in need thereof a lipid nanoparticle (LNP) composition comprising a nucleic acid signal regulatory protein alpha (SIRPα) therapeutic.

67. The method of claim 66, wherein the SIRPα therapeutic comprises a polynucleotide having at least 80% identity to a contiguous sequence within SEQ ID NO:4, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17, or a combination thereof, wherein the contiguous nucleotide sequence is at least 15 nucleotides in length.

68. The method of claim 67, wherein the polynucleotide sequence further comprises at least one modified pyrimidine, and the at least one modified pyrimidine is 2′-O-methylcytidine-3′-phosphate or 2′-O-methyluridine-3′-phosphate.

69. The method of claim 68, wherein the SIRPα therapeutic is an siRNA duplex.

70. The method of claim 69, wherein the lipid nanoparticle (LNP) comprises an ionizable lipid, phospholipid, sterol, polymer conjugated lipid, or any combination thereof.

71. The method of claim 70, wherein the ionizable lipid is selected from the group consisting of FL-A; MC3; Lipid 5; Lipid A9; ALC-0315; and GCL1.

72. The method of claim 71, wherein the SIRPα-mediated disease or condition is selected from the group consisting of acute myeloid leukemia, adenosquamous lung carcinoma, atypical meningioma, B-cell acute lymphoblastic leukemia, basal cell carcinoma, bile duct carcinoma, bladder carcinoma, bladder transitional cell carcinoma, brain glioblastoma, breast carcinoma, breast ductal adenocarcinoma, Burkitts lymphoma, cecum adenocarcinoma, cervical squamous cell carcinoma, chronic lymphosytic leukemia, clear cell renal carcinoma, colon adenocarcinoma, cutaneous melanoma, endometrial endometrioid adenocarcinoma, esophageal adenocarcinoma, esophageal squamous cell carcinoma, gastric adenocarcinoma, gastric carcinoma, glioma, head and neck squamous cell carcinoma, hepatobiliary neoplasm, hepatoblastoma, hepatocellular carcina, HER2 positive breast carcinoma, kidney neoplasm, large cell lung carcinoma, lobular breast carcinoma, lunch adenocarcinoma, lung carcinoma, lymphoid neoplasm, melanoma, multiple myeloma, nasopharyngeal squamous cell carcinoma, non-small cell lung carcinoma, oral squamous cell carcinoma, ovarian endometroid adenocarcinoma with squamous differentiation, ovarian serous adenocarcinoma, pancreatic acinar cell carcinoma, pancreatic carcinoma, pancreatic ductal adenocarcinoma, pancreatic neuroendocrine tumor, papillary renal cell carcinoma, prostate adenocarcinoma, rectal adenocarcinoma, skin carcinoma, small cell lung carcinoma, squamous cell lung carcinoma, thyroid carcinoma, thyroid neoplasm, and uterine carcinosarcoma.

73. A method of making a lipid nanoparticle formulation comprising:
a) obtaining a signal regulatory protein alpha (SIRPα) therapeutic, wherein the SIRPα therapeutic comprises a polynucleotide having at least 80% identity to a contiguous sequence SEQ ID NO:4, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17;
b) diluting the polynucleotide in citrate buffer to form an aqueous phase;
c) solubilizing a mixture of ionizable lipid, 1,2-distearoyl-sn-glycero-3-phosphocholine (DSPC), cholesterol and 1,2-dimyristoyl-rac-glycero-3-methoxypolyethylene glycol-2000 (DMG-PEG2000) to form an ethanol phase;
d) mixing the aqueous phase with the ethanol phase, wherein a precipitate is formed;
e) separating the precipitate to obtain a lipid nanoparticle formulation.
